SEQUENCE LISTING (15 sequences)

SEQ ID NO: 1 - DNA, 505 bp, Zea mays
tagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgca

SEQ ID NO: 2 - DNA, 543 bp, Zea mays
cccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagct

SEQ ID NO: 3 - DNA, 51280 bp, Artificial Sequence
  plasmid as shown in Figure 1
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggataagtgactagggtcacgtgaccctagtcacttatcgagctagttaccctatgaggtgacatgaagcgctcacggttactatgacggttagcttcacgactgttggtggcagtagcgtacgacttagctatagttccggtagatctgaagttcctattccgaagttcctattcttcaaaaggtataggaacttcctcgaattgttgtggtggggtatagaggtttgatataggtggaactgctgtagagcgtggagatatagggggaaagagaacgctgatgtgacaagtgagtgagatatagggggagaaatttagggggaacgccgaacacagtctaaagaagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatcaccggttaagtgactagggtcacgtgaccctagtcacttaggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcacaagtttgtacaaaaaagcaggctccggccagagttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccactagtaacggccgccagtgtgctggaattcgccctttagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagctcgaattccggtccgggtcacccggtccgggcctagaaggccgatctcccgggcacccagctttcttgtacaaagtggtgatatcggaccgattaaactttaattcggtccgatgcatgtatacgaagttcctattccgaagttcctattctacatagagtataggaacttcacctggtggcgccgctagtggatcccccgggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgctctagcgtatacgaagttcctattccgaagttcctattctctagaaagtataggaacttctgattccgatgacttcgtaggttcctagctcaagccgctcgtgtccaagcgtcacttacgattagctaatgattacggcatctaggaccgactagtaagtgactagggtcacgtgaccctagtcacttatacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacagtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcggagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccactcttcattaaccgctatatcgaaaattgcttgcggcttgttagaattgccatgacgtacctcggtgtcacgggtaagattaccgataaactggaactgattatggctcatatcgaaagtctccttgagaaaggagactctagtttagctaaacattggttccgctgtcaagaactttagcggctaaaattttgcgggccgcgaccaaaggtgcgaggggcggcttccgctgtgtacaaccagatatttttcaccaacatccttcgtctgctcgatgagcggggcatgacgaaacatgagctgtcggagagggcaggggtttcaatttcgtttttatcagacttaaccaacggtaaggccaacccctcgttgaaggtgatggaggccattgccgacgccctggaaactcccctacctcttctcctggagtccaccgaccttgaccgcgaggcactcgcggagattgcgggtcatcctttcaagagcagcgtgccgcccggatacgaacgcatcagtgtggttttgccgtcacataaggcgtttatcgtaaagaaatggggcgacgacacccgaaaaaagctgcgtggaaggctctgacgccaagggttagggcttgcacttccttctttagccgctaaaacggccccttctctgcgggccgtcggctcgcgcatcatatcgacatcctcaacggaagccgtgccgcgaatggcatcgggcgggtgcgctttgacagttgttttctatcagaacccctacgtcgtgcggttcgattagctgtttgtcttgcaggctaaacactttcggtatatcgtttgcctgtgcgataatgttgctaatgatttgttgcgtaggggttactgaaaagtgagcgggaaagaagagtttcagaccatcaaggagcgggccaagcgcaagctggaacgcgacatgggtgcggacctgttggccgcgctcaacgacccgaaaaccgttgaagtcatgctcaacgcggacggcaaggtgtggcacgaacgccttggcgagccgatgcggtacatctgcgacatgcggcccagccagtcgcaggcgattatagaaacggtggccggattccacggcaaagaggtcacgcggcattcgcccatcctggaaggcgagttccccttggatggcagccgctttgccggccaattgccgccggtcgtggccgcgccaacctttgcgatccgcaagcgcgcggtcgccatcttcacgctggaacagtacgtcgaggcgggcatcatgacccgcgagcaatacgaggtcattaaaagcgccgtcgcggcgcatcgaaacatcctcgtcattggcggtactggctcgggcaagaccacgctcgtcaacgcgatcatcaatgaaatggtcgccttcaacccgtctgagcgcgtcgtcatcatcgaggacaccggcgaaatccagtgcgccgcagagaacgccgtccaataccacaccagcatcgacgtctcgatgacgctgctgctcaagacaacgctgcgtatgcgccccgaccgcatcctggtcggtgaggtacgtggccccgaagcccttgatctgttgatggcctggaacaccgggcatgaaggaggtgccgccaccctgcacgcaaacaaccccaaagcgggcctgagccggctcgccatgcttatcagcatgcacccggattcaccgaaacccattgagccgctgattggcgaggcggttcatgtggtcgtccatatcgccaggacccctagcggccgtcgagtgcaagaaattctcgaagttcttggttacgagaacggccagtacatcaccaaaaccctgtaaggagtatttccaatgacaacggctgttccgttccgtctgaccatgaatcgcggcattttgttctaccttgccgtgttcttcgttctcgctctcgcgttatccgcgcatccggcgatggcctcggaaggcaccggcggcagcttgccatatgagagctggctgacgaacctgcgcaactccgtaaccggcccggtggccttcgcgctgtccatcatcggcatcgtcgtcgccggcggcgtgctgatcttcggcggcgaactcaacgccttcttccgaaccctgatcttcctggttctggtgatggcgctgctggtcggcgcgcagaacgtgatgagcaccttcttcggtcgtggtgccgaaatcgcggccctcggcaacggggcgctgcaccaggtgcaagtcgcggcggcggatgccgtgcgtgcggtagcggctggacggctcgcctaatcatggctctgcgcacgatccccatccgtcgcgcaggcaaccgagaaaacctgttcatgggtggtgatcgtgaactggtgatgttctcgggcctgatggcgtttgcgctgattttcagcgcccaagagctgcgggccaccgtggtcggtctgatcctgtggttcggggcgctctatgcgttccgaatcatggcgaaggccgatccgaagatgcggttcgtgtacctgcgtcaccgccggtacaagccgtattacccggcccgctcgaccccgttccgcgagaacaccaatagccaagggaagcaataccgatgatccaagcaattgcgattgcaatcgcgggcctcggcgcgcttctgttgttcatcctctttgcccgcatccgcgcggtcgatgccgaactgaaactgaaaaagcatcgttccaaggacgccggcctggccgatctgctcaactacgccgctgtcgtcgatgacggcgtaatcgtgggcaagaacggcagctttatggctgcctggctgtacaagggcgatgacaacgcaagcagcaccgaccagcagcgcgaagtagtgtccgcccgcatcaaccaggccctcgcgggcctgggaagtgggtggatgatccatgtggacgccgtgcggcgtcctgctccgaactacgcggagcggggcctgtcggcgttccctgaccgtctgacggcagcgattgaagaagagcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatctactgtaatggcgaaaattcacatggttttgcagggcaagggcggggtcggcaagtcggccatcgccgcgatcattgcgcagtacaagatggacaaggggcagacacccttgtgcatcgacaccgacccggtgaacgcgacgttcgagggctacaaggccctgaacgtccgccggctgaacatcatggccggcgacgaaattaactcgcgcaacttcgacaccctggtcgagctgattgcgccgaccaaggatgacgtggtgatcgacaacggtgccagctcgttcgtgcctctgtcgcattacctcatcagcaaccaggtgccggctctgctgcaagaaatggggcatgagctggtcatccataccgtcgtcaccggcggccaggctctcctggacacggtgagcggcttcgcccagctcgccagccagttcccggccgaagcgcttttcgtggtctggctgaacccgtattgggggcctatcgagcatgagggcaagagctttgagcagatgaaggcgtacacggccaacaaggcccgcgtgtcgtccatcatccagattccggccctcaaggaagaaacctacggccgcgatttcagcgacatgctgcaagagcggctgacgttcgaccaggcgctggccgatgaatcgctcacgatcatgacgcggcaacgcctcaagatcgtgcggcgcggcctgtttgaacagctcgacgcggcggccgtgctatgagcgaccagattgaagagctgatccgggagattgcggccaagcacggcatcgccgtcggccgcgacgacccggtgctgatcctgcataccatcaacgcccggctcatggccgacagtgcggccaagcaagaggaaatccttgccgcgttcaaggaagagctggaagggatcgcccatcgttggggcgaggacgccaaggccaaagcggagcggatgctgaacgcggccctggcggccagcaaggacgcaatggcgaaggtaatgaaggacagcgccgcgcaggcggccgaagcgatccgcagggaaatcgacgacggccttggccgccagctcgcggccaaggtcgcggacgcgcggcgcgtggcgatgatgaacatgatcgccggcggcatggtgttgttcgcggccgccctggtggtgtgggcctcgttatgaatcgcagaggcgcagatgaaaaagcccggcgttgccgggctttgtttttgcgttagctgggcttgtttgacaggcccaagctctgactgcgcccgcgctcgcgctcctgggcctgtttcttctcctgctcctgcttgcgcatcagggcctggtgccgtcgggctgcttcacgcatcgaatcccagtcgccggccagctcgggatgctccgcgcgcatcttgcgcgtcgccagttcctcgatcttgggcgcgtgaatgcccatgccttccttgatttcgcgcaccatgtccagccgcgtgtgcagggtctgcaagcgggcttgctgttgggcctgctgctgctgccaggcggcctttgtacgcggcagggacagcaagccgggggcattggactgtagctgctgcaaacgcgcctgctgacggtctacgagctgttctaggcggtcctcgatgcgctccacctggtcatgctttgcctgcacgtagagcgcaagggtctgctggtaggtctgctcgatgggcgcggattctaagagggcctgctgttccgtctcggcctcctgggccgcctgtagcaaatcctcgccgctgttgccgctggactgctttactgccggggactgctgttgccctgctcgcgccgtcgtcgcagttcggcttgcccccactcgattgactgcttcatttcgagccgcagcgatgcgatctcggattgcgtcaacggacggggcagcgcggaggtgtccggcttctccttgggtgagtcggtcgatgccatagccaaaggtttccttccaaaatgcgtccattgctggaccgtgtttctcattgatgcccgcaagcatcttcggcttgaccgccaggtcaagcgcgccttcatgggcggtcatgacggacgccgccatgaccttgccgccgttgttctcgatgtagccgcgtaatgaggcaatggtgccgcccatcgtcagcgtgtcatcgacaacgatgtacttctggccggggatcacctccccctcgaaagtcgggttgaacgccaggcgatgatctgaaccggctccggttcgggcgaccttctcccgctgcacaatgtccgtttcgacctcaaggccaaggcggtcggccagaacgaccgccatcatggccggaatcttgttgttccccgccgcctcgacggcgaggactggaacgatgcggggcttgtcgtcgccgatcagcgtcttgagctgggcaacagtgtcgtccgaaatcaggcgctcgaccaaattaagcgccgcttccgcgtcgccctgcttcgcagcctggtattcaggctcgttggtcaaagaaccaaggtcgccgttgcgaaccaccttcgggaagtctccccacggtgcgcgctcggctctgctgtagctgctcaagacgcctccctttttagccgctaaaactctaacgagtgcgcccgcgactcaacttgacgctttcggcacttacctgtgccttgccacttgcgtcataggtgatgcttttcgcactcccgatttcaggtactttatcgaaatctgaccgggcgtgcattacaaagttcttccccacctgttggtaaatgctgccgctatctgcgtggacgatgctgccgtcgtggcgctgcgacttatcggccttttgggccatatagatgttgtaaatgccaggtttcagggccccggctttatctaccttctggttcgtccatgcgccttggttctcggtctggacaattctttgcccattcatgaccaggaggcggtgtttcattgggtgactcctgacggttgcctctggtgttaaacgtgtcctggtcgcttgccggctaaaaaaaagccgacctcggcagttcgaggccggctttccctagagccgggcgcgtcaaggttgttccatctattttagtgaactgcgttcgatttatcagttactttcctcccgctttgtgtttcctcccactcgtttccgcgtctagccgacccctcaacatagcggcctcttcttgggctgcctttgcctcttgccgcgcttcgtcacgctcggcttgcaccgtcgtaaagcgctcggcctgcctggccgcctcttgcgccgccaacttcctttgctcctggtgggcctcggcgtcggcctgcgccttcgctttcaccgctgccaactccgtgcgcaaactctccgcttcgcgcctggtggcgtcgcgctcgccgcgaagcgcctgcatttcctggttggccgcgtccagggtcttgcggctctcttctttgaatgcgcgggcgtcctggtgagcgtagtccagctcggcgcgcagctcctgcgctcgacgctccacctcgtcggcccgctgcgtcgccagcgcggcccgctgctcggctcctgccagggcggtgcgtgcttcggccagggcttgccgctggcgtgcggccagctcggccgcctcggcggcctgctgctctagcaatgtaacgcgcgcctgggcttcttccagctcgcgggcctgcgcctcgaaggcgtcggccagctccccgcgcacggcttccaactcgttgcgctcacgatcccagccggcttgcgctgcctgcaacgattcattggcaagggcctgggcggcttgccagagggcggccacggcctggttgccggcctgctgcaccgcgtccggcacctggactgccagcggggcggcctgcgccgtgcgctggcgtcgccattcgcgcatgccggcgctggcgtcgttcatgttgacgcgggcggccttacgcactgcatccacggtcgggaagttctcccggtcgccttgctcgaacagctcgtccgcagccgcaaaaatgcggtcgcgcgtctctttgttcagttccatgttggctccggtaattggtaagaataataatactcttacctaccttatcagcgcaagagtttagctgaacagttctcgacttaacggcaggttttttagcggctgaagggcaggcaaaaaaagccccgcacggtcggcgggggcaaagggtcagcgggaaggggattagcgggcgtcgggcttcttcatgcgtcggggccgcgcttcttgggatggagcacgacgaagcgcgcacgcgcatcgtcctcggccctatcggcccgcgtcgcggtcaggaacttgtcgcgcgctaggtcctccctggtgggcaccaggggcatgaactcggcctgctcgatgtaggtccactccatgaccgcatcgcagtcgaggccgcgttccttcaccgtctcttgcaggtcgcggtacgcccgctcgttgagcggctggtaacgggccaattggtcgtaaatggctgtcggccatgagcggcctttcctgttgagccagcagccgacgacgaagccggcaatgcaggcccctggcacaaccaggccgacgccgggggcaggggatggcagcagctcgccaaccaggaaccccgccgcgatgatgccgatgccggtcaaccagcccttgaaactatccggccccgaaacacccctgcgcattgcctggatgctgcgccggatagcttgcaacatcaggagccgtttcttttgttcgtcagtcatggtccgccctcaccagttgttcgtatcggtgtcggacgaactgaaatcgcaagagctgccggtatcggtccagccgctgtccgtgtcgctgctgccgaagcacggcgaggggtccgcgaacgccgcagacggcgtatccggccgcagcgcatcgcccagcatggccccggtcagcgagccgccggccaggtagcccagcatggtgctgttggtcgccccggccaccagggccgacgtgacgaaatcgccgtcattccctctggattgttcgctgctcggcggggcagtgcgccgcgccggcggcgtcgtggatggctcgggttggctggcctgcgacggccggcgaaaggtgcgcagcagctcgttatcgaccggctgcggcgtcggggccgccgccttgcgctgcggtcggtgttccttcttcggctcgcgcagcttgaacagcatgatcgcggaaaccagcagcaacgccgcgcctacgcctcccgcgatgtagaacagcatcggattcattcttcggtcctccttgtagcggaaccgttgtctgtgcggcgcgggtggcccgcgccgctgtctttggggatcagccctcgatgagcgcgaccagtttcacgtcggcaaggttcgcctcgaactcctggccgtcgtcctcgtacttcaaccaggcatagccttccgccggcggccgacggttgaggataaggcgggcagggcgctcgtcgtgctcgacctggacgatggcctttttcagcttgtccgggtccggctccttcgcgcccttttccttggcgtccttaccgtcctggtcgccgtcctcgccgtcctggccgtcgccggcctccgcgtcacgctcggcatcagtctggccgttgaaggcatcgacggtgttgggatcgcggcccttctcgtccaggaactcgcgcagcagcttgaccgtgccgcgcgtgatttcctgggtgtcgtcgtcaagccacgcctcgacttcctccgggcgcttcttgaaggccgtcaccagctcgttcaccacggtcacgtcgcgcacgcggccggtgttgaacgcatcggcgatcttctccggcaggtccagcagcgtgacgtgctgggtgatgaacgccggcgacttgccgatttccttggcgatatcgcctttcttcttgcccttcgccagctcgcggccaatgaagtcggcaatttcgcgcggggtcagctcgttgcgttgcaggttctcgataacctggtcggcttcgttgtagtcgttgtcgatgaacgccgggatggacttcttgccggcccacttcgagccacggtagcggcgggcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgctgaattgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctggaagccccgtagcgacgcggagaggggcgagacaagccaagggcgcaggctcgatgcgcagcacgacatagccggttctcgcaaggacgagaatttccctgcggtgcccctcaagtgtcaatgaaagtttccaacgcgagccattcgcgagagccttgagtccacgctagatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgactctagagctcgttcctcgaggcctcgaggcctcgaggaacggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtactgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtaggtttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatttagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggtgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccatcgatagatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtactgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttag

SEQ ID NO: 4 - DNA, 1603 bp, Artificial Sequence
  synthesized
tagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagct

SEQ ID NO: 5 - DNA, 3657 bp, Artificial Sequence
  synthesized
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccactagtaacggccgccagtgtgctggaattcgccctttagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagct

SEQ ID NO: 6 - DNA, 6772 bp, Artificial Sequence
  synthesized
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccactagtaacggccgccagtgtgctggaattcgccctttagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagctcgaattccggtccgggtcacccggtccgggcctagaaggccgatctcccgggcacccagctttcttgtacaaagtggtgatatcggaccgattaaactttaattcggtccgatgcatgtatacgaagttcctattccgaagttcctattctacatagagtataggaacttcacctggtggcgccgctagtggatcccccgggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcc

SEQ ID NO: 7 - DNA, 8350 bp, Artificial Sequence
  synthesized
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggataagtgactagggtcacgtgaccctagtcacttatcgagctagttaccctatgaggtgacatgaagcgctcacggttactatgacggttagcttcacgactgttggtggcagtagcgtacgacttagctatagttccggtagatctgaagttcctattccgaagttcctattcttcaaaaggtataggaacttcctcgaattgttgtggtggggtatagaggtttgatataggtggaactgctgtagagcgtggagatatagggggaaagagaacgctgatgtgacaagtgagtgagatatagggggagaaatttagggggaacgccgaacacagtctaaagaagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatcaccggttaagtgactagggtcacgtgaccctagtcacttaggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcacaagtttgtacaaaaaagcaggctccggccagagttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccactagtaacggccgccagtgtgctggaattcgccctttagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagctcgaattccggtccgggtcacccggtccgggcctagaaggccgatctcccgggcacccagctttcttgtacaaagtggtgatatcggaccgattaaactttaattcggtccgatgcatgtatacgaagttcctattccgaagttcctattctacatagagtataggaacttcacctggtggcgccgctagtggatcccccgggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgctctagcgtatacgaagttcctattccgaagttcctattctctagaaagtataggaacttctgattccgatgacttcgtaggttcctagctcaagccgctcgtgtccaagcgtcacttacgattagctaatgattacggcatctaggaccgactagtaagtgactagggtcacgtgaccctagtcacttatacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 8 - DNA, 1424 bp, Sorghum bicolor
  promoter (1)...(1424): sorghum PEP carboxylase promoter
ggacatgtaataaggagttaggagatgtggtgtggtactaaatgcaaggtcaaaattcgatgctttttccgtgctcaactattaactagtactcattattacctaattttcacttgtgatgacaattaatgcatcgatccacaattcagtaaatactttcatttaagcatatgtatagtattatacatttccaattcttcttttttgtgtggagatccacgacgatgcaagttgctcctcccaacccaaatccacctctctcttaaatccgcatatcttcaccaccaccagctgctacacatcgtattgtccaaatctgtgtcggcttgacccagtgatgtgcgcgctagatttggcagcgcctgaatgctgtgcagccacctgtatggtgcccttggtagagtaacaacacccttatccctacggcagccatgtatgacccttatccctacggcagccatgtataccaatacctttctttgaaccacaaaattatagtccatatccttaaccacaagttcattttttgtttcccggtctcctaaggaaattaagttctgtttccacagtttacatggatataggacatctatgttcctaacattaacattactggataacaggcaccctctcctccacaccctgcaaagccttcctccagcgccatgcatcctccgttgctaacagacacctctctccacatcgcgtgcaagcaaacctccaaattctaccgatccccagaatccggccttgactgcaaacagacacccctctccccatcctgcaaacccatcagccaaccgaataacacaagaaggcaggtgagcagtgacaaagcacgtcaacagcagcaaagccaagccaaaaacgatccaggagcaaggtgcggccgcagctctcccggtcccctttgcggttaccactagctaagaatgaagatggtactctaaatggatacttgcgcggtttttctctagtctaacttaataaactaaataaacaatttctttcttatttttttaatttagttcgtttagttagactagagaagaaccacgaggagttatttgaagcgtcgtccccatccttaccactagctagcactagcagacacccctctccacgtcctgcaaacaggcaattagccagcggaataacacaagcaggcaagtgcgcagtgacaaagtacgtccacagcagcgatcccagccaaaagcagcgtagccacagccgcgcgcagctctcggctacccttaccgccgatcacatgcatgcctttccaatcccgcgtgcacacgccgaccacacactcgccaactccccatccctatttgaagccaccggccggcgccctgcattgatcaatcaactcgcagcagaggagcagcacgagcaacacgccgcgccgcgctccaaccatctcc

SEQ ID NO: 9 - DNA, 2765 bp, Zea mays
  misc_feature (1)...(2765): genomic ACS3 sequence
tagtagactaaagctcccggctttccctttattgttactattattatatcgtttgatttgcagcggacagtctggtcgtttccgtctgctctggaccctgccttcaattctccgccttttgacatcttgggtggtttcacacgggtatatatacttgactcgcctatatatgccaccccccacacacttggccagatcgaccaccgcatcacacagcagcagcctcttgtgctcctctctgtcctttgcttgctcatccctcgacctcgagcggcggcggcggtcactgcactgcgtgtgcgtatccatccatcttgcaggcatcgtcgtctgcaccgcgcgcttgtgcgtgagccaagggcggcagaggcgatgggtggcaagctgttgctgggcgcgagccagagccgccacgcgcacgcggtggcgtcgcctcccctgtcaaaggtggccacttccggcctccacggcgaggactcgccctacttcgccgggtggaaagcctacgacgagaacccctacgacgccgtctccaaccccggcggcgtcattcagatgggcctcgccgagaaccaggtgtccttcgacctcctcgaggggtacctcagggaccacccggaggccgcgggctggggcggctccggctccggcgtcgccagcttcagggacaacgcgctgttccaggactaccacggcctcaaggccttcagaaaggtgcgtggtgaccccgagcgcctgctctgctcgtcttgttcggtgtcctggatagaccctgcctggacaagtgcaacgtactgacggcgtcattttgcttgcaggcgatggccaacttcatggagaaggttaggggcggcaaggcccggtttgaccccgaccgcatcgtgctcaccgccggcgccacggcagctaacgagctgctcacgttcgtcctggccaacccgggagacgcgctgctgatccctactccttactatcctgggtaagcggagcgctaagaagcatccagcatgctgcatgcatgcacggacccccggcgctatgctactgttacaccgcaacagtgctctacacagacagacagacaaactgacatgctctgcttgcagtttcgacagagacctgcggtggaggacgggggtgaacatcgtgccggtgcactgcgacagcgccaacgggttccaggtcacggccgccgcgctccaggcggcgtacgaggaggccgaggcagcggggacgcgcgtccgcgccgtcctgctcaccaacccgtccaacccgctgggcaccaccgtgacgcggccggccctcgaggacgtgctcgacttcgtggcccgcaacaacatccacctcatctccgacgagatatactccggctcggtcttcgcggcgccggacctggtcagcgtggcggagctggtcgagtcccgcggcgaccccggcgtcgcggagcgcgtccacatcgtgtacagcctgtccaaggacctgggcctcccggggttccgcgtcggcgtcgtgtactcgtacaacgacgccgtggtcaccgcggcgcgccgcatgtccagcttcacgctcgtgtcgtcgcagacgcagaagacgctcgccgccatgctctcggacgccgggttcgcggacgcctacgtccgcaccaaccgccagcgcctccgggcgcggcacgaccacgtcgtcgccgggctggcccgcgcgggcgtgccgtgcctccgcggcaacgccgggctgttcgtgtggatggacatgaggcggctgctcggcgaggccaccaccgtcgccggcgagctccgcctgtgggaccggatgctgcgggaggcgaagctcaacatctcgccgggctcgtcgtgccattgctcggagcctggctggttcagggtgtgcttcgccaacatgagcctggacacgctggatgttgcactcgctaggatgagccgcttcgtagacacgtggaacaaggaaacgacagcgtcgacgcagcagcactagcagcagcagcagcatacgaagtaaattttttggagggtaaattacgtcattggacagattaaatcacagagtagttatacagggggattcttttatggtttttcgattgatggtaacatcgattttgtaacaataactatcgcctctcagatggaggagggacacatatatgtatgtatttataaaaattcttactttggcccaagcaaaagcgtctccgctacacattcagtattattgttttgcttcgtttttccctcctgagttgtggcaaacaaacacagatgacgtatgtttgctcattcattcatatatttatatctgcctggaagcgaacctaaattagcaacaagagagaacatgtccattccgagtactgagaataagtgcagcagaatgaatgctggttgttggatttaaattagatggatgttgtttgtcgtcagaaaatggagagagagaaatcagttagcgaatccctttctcgttttatactgctggttttctatacttttgaagaaaaaaaactgttttgctgaggtgttcgatgttgtaaattactgattgacttcatgtgattgcgtaacatgttactggacggaggtttacttctatacatgagtagtgtcataccaaaccaaaaaaaaaagggaatcgaaagtcttgatagcggacgtgttgattaaagaaaaagaaaccgagttccaaatgtcaacccttccttaagccggtagtaagctttgtcaaccagctaaataagggaacgcatatcc

SEQ ID NO: 10 - DNA, 1759 bp, Zea mays
  CDS (160)...(1548): ACS3
cagcagcagcctcttgtgctcctctctgtcctttgcttgctcatccctcgacctcgagcggcggcggcggtcactgcactgcgtgtgcgtatccatccatcttgcaggcatcgtcgtctgcaccgcgcgcttgtgcgtgagccaagggcggcagaggcgatgggtggcaagctgttgctgggcgcgagccagagccgccacgcgcacgcggtggcgtcgcctcccctgtcaaaggtggccacttccggcctccacggcgaggactcgccctacttcgccgggtggaaagcctacgacgagaacccctacgacgccgtctccaaccccggcggcgtcattcagatgggcctcgccgagaaccaggtgtccttcgacctcctcgaggggtacctcagggaccacccggaggccgcgggctggggcggctccggctccggcgtcgccagcttcagggacaacgcgctgttccaggactaccacggcctcaaggccttcagaaaggcgatggccaacttcatggagaaggttaggggcggcaaggcccggtttgaccccgaccgcatcgtgctcaccgccggcgccacggcagctaacgagctgctcacgttcgtcctggccaacccgggagacgcgctgctgatccctactccttactatcctggtttcgacagagacctgcggtggaggacgggggtgaacatcgtgccggtgcactgcgacagcgccaacgggttccaggtcacggccgccgcgctccaggcggcgtacgaggaggccgaggcagcggggacgcgcgtccgcgccgtcctgctcaccaacccgtccaacccgctgggcaccaccgtgacgcggccggccctcgaggacgtgctcgacttcgtggcccgcaacaacatccacctcatctccgacgagatatactccggctcggtcttcgcggcgccggacctggtcagcgtggcggagctggtcgagtcccgcggcgaccccggcgtcgcggagcgcgtccacatcgtgtacagcctgtccaaggacctgggcctcccggggttccgcgtcggcgtcgtgtactcgtacaacgacgccgtggtcaccgcggcgcgccgcatgtccagcttcacgctcgtgtcgtcgcagacgcagaagacgctcgccgccatgctctcggacgccgggttcgcggacgcctacgtccgcaccaaccgccagcgcctccgggcgcggcacgaccacgtcgtcgccgggctggcccgcgcgggcgtgccgtgcctccgcggcaacgccgggctgttcgtgtggatggacatgaggcggctgctcggcgaggccaccaccgtcgccggcgagctccgcctgtgggaccggatgctgcgggaggcgaagctcaacatctcgccgggctcgtcgtgccattgctcggagcctggctggttcagggtgtgcttcgccaacatgagcctggacacgctggatgttgcactcgctaggatgagccgcttcgtagacacgtggaacaaggaaacgacagcgtcgacgcagcagcactagcagcagcagcagcatacgaagtaaattttttggagggtaaattacgtcattggacagattaaatcacagagtagttatacagggggattcttttatggtttttcgattgatggtaacatcgattttgtaacaataactatcgcctctcagatggaggagggacacatatatgtatgtatttataaaaattcttactttggcccaagcaaaa

SEQ ID NO: 11 - protein, 462 aa, Zea mays
MGGKLLLGASQSRHAHAVASPPLSKVATSGLHGEDSPYFAGWKAYDENPYDAVSNPGGVIQMGLAENQVSFDLLEGYLRDHPEAAGWGGSGSGVASFRDNALFQDYHGLKAFRKAMANFMEKVRGGKARFDPDRIVLTAGATAANELLTFVLANPGDALLIPTPYYPGFDRDLRWRTGVNIVPVHCDSANGFQVTAAALQAAYEEAEAAGTRVRAVLLTNPSNPLGTTVTRPALEDVLDFVARNNIHLISDEIYSGSVFAAPDLVSVAELVESRGDPGVAERVHIVYSLSKDLGLPGFRVGVVYSYNDAVVTAARRMSSFTLVSSQTQKTLAAMLSDAGFADAYVRTNRQRLRARHDHVVAGLARAGVPCLRGNAGLFVWMDMRRLLGEATTVAGELRLWDRMLREAKLNISPGSSCHCSEPGWFRVCFANMSLDTLDVALARMSRFVDTWNKETTASTQQH

SEQ ID NO: 12 - DNA, 1464 bp, Oryza sativa
  CDS (1)...(1461)
atggtgagccaagtggtcgccgaggagaagccgcagctgctgtccaagaaggccgggtgcaacagccacggccaggactcgtcctacttcctggggtggcaggagtacgagaaaaacccgttcgaccccgtctccaacccttccggcatcatccagatgggcctcgccgagaaccagctgtcgttcgacctgcttgaggagtggctggagaagaacccccacgcgctcggcctccggcgagagggcggcggcgcctccgtcttccgcgagctcgcgctgttccaggactaccacggcctcccggctttcaaaaatgcattggcgcggttcatgtcggagcagagagggtacaaggtggtgttcgaccccagcaacatcgtgctcaccgccggcgccacctcggctaacgaggcgctcatgttctgcctcgccgaccacggcgacgccttcctcatccccaccccatactacccagggttcgaccgcgacctcaagtggcgcaccggcgcggagatcgtacccgtgcactgcgcgagcgcgaacgggttccgggtgacgcgccccgcgctggacgacgcgtaccgccgcgcgcagaagcgccggctgcgcgtcaagggggtgctgatcaccaacccgtccaacccgctcggcaccgcgtcgccgcgcgccgacctcgagacgatcgtcgacttcgtcgccgccaagggcatccacctcatcagcgacgagatctacgccggcacggcgttcgccgagccgcccgcgggcttcgtcagcgcgctcgaggtcgtggccgggcgcgacggcggcggcgctgacgtgtccgaccgcgtgcacgtcgtgtacagcctgtccaaggacctcggcctcccggggttccgcgtcggcgccatctactccgccaacgccgccgtcgtgtccgcggcgaccaagatgtccagcttcggcctcgtgtcgtcccagacgcagtacctcctcgcggcgctgctcggcgacagggacttcacccggagctacgtcgcggagaacaagcggcggatcaaggagcggcacgaccagctcgtggacgggctcagggagatcggcattgggtgcctgcccagcaacgccggcctcttctgctgggtggacatgagccacctgatgcggagccggtcgttcgccggcgagatggagctctggaagaaggtggtgttcgaggtcggcctcaacatctcccccgggtcgtcgtgccactgccgcgagcccggctggttccgcgtctgcttcgccaacatgtcggccaagaccctcgacgtcgccatgcagcgcctcaggtcgttcgtcgactccgccaccggcggcggcgacaacgccgccctccgccgcgccgccgtccccgtcaggagcgtcagctgcccgctcgccatcaagtgggcgctccgcctcaccccgtccatcgccgaccggaaggccgagagataa

SEQ ID NO: 13 - protein, 487 aa, Oryza sativa
MVSQVVAEEKPQLLSKKAGCNSHGQDSSYFLGWQEYEKNPFDPVSNPSGIIQMGLAENQLSFDLLEEWLEKNPHALGLRREGGGASVFRELALFQDYHGLPAFKNALARFMSEQRGYKVVFDPSNIVLTAGATSANEALMFCLADHGDAFLIPTPYYPGFDRDLKWRTGAEIVPVHCASANGFRVTRPALDDAYRRAQKRRLRVKGVLITNPSNPLGTASPRADLETIVDFVAAKGIHLISDEIYAGTAFAEPPAGFVSALEVVAGRDGGGADVSDRVHVVYSLSKDLGLPGFRVGAIYSANAAVVSAATKMSSFGLVSSQTQYLLAALLGDRDFTRSYVAENKRRIKERHDQLVDGLREIGIGCLPSNAGLFCWVDMSHLMRSRSFAGEMELWKKVVFEVGLNISPGSSCHCREPGWFRVCFANMSAKTLDVAMQRLRSFVDSATGGGDNAALRRAAVPVRSVSCPLAIKWALRLTPSIADRKAER

SEQ ID NO: 14 - DNA, 1446 bp, Zea mays
atgatcgccgacgagaagccgcagccgcagctgctgtccaagaaggccgcctgcaacagccacggccaggactcgtcctacttcctggggtgggaggagtatgagaaaaacccatacgaccccgtcgccaaccccggcggcatcatccagatgggcctcgccgagaaccagctgtccttcgacctgctggaggcgtggctggaggccaacccggacgcgctcggcctccgccggggaggcgcctctgtattccgcgagctcgcgctcttccaggactaccacggcatgccggccttcaagaatgcattggcgaggttcatgtcggagcaacgtgggtaccgggtgaccttcgaccccagcaacatcgtgctcaccgccggagccacctcggccaacgaggccctcatgttctgcctcgccgaccacggagacgcctttctcatccccacgccatactacccagggttcgaccgtgacctcaagtggcgcaccggcgcggagatcgtccccgtgcactgcacgagcggcaacggcttccggctgacgcgcgccgcgctggacgacgcgtaccggcgcgcgcagaagctgcggctgcgcgtcaagggcgtgctcatcaccaacccttccaacccgctgggcaccacgtcgccgcgcgccgacctggagatgctggtggacttcgtggccgccaagggcatccacctggtgagcgacgagatatactcgggcacggtcttcgcggacccgggcttcgtgagcgtcctcgaggtggtggccgcgcgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagaaggtggtcttcgaggtggggctcaacatctccccgggctcctcctgccactgccgggagcccggctggttccgcgtctgcttcgccaacatgtccgccaagacgctcgacgtcgcgctccagcgcctgggcgccttcgcggaggccgccaccgcggggcgccgcgtgcttgcccccgccaggagcatcagcctcccggtccgcttcagctgggctaaccgcctcaccccgggctccgccgccgaccggaaggccgagcgg

SEQ ID NO: 15 - protein, 482 aa, Zea mays
MIADEKPQPQLLSKKAACNSHGQDSSYFLGWEEYEKNPYDPVANPGGIIQMGLAENQLSFDLLEAWLEANPDALGLRRGGASVFRELALFQDYHGMPAFKNALARFMSEQRGYRVTFDPSNIVLTAGATSANEALMFCLADHGDAFLIPTPYYPGFDRDLKWRTGAEIVPVHCTSGNGFRLTRAALDDAYRRAQKLRLRVKGVLITNPSNPLGTTSPRADLEMLVDFVAAKGIHLVSDEIYSGTVFADPGFVSVLEVVAARAATDDGVVGVGPLSDRVHVVYSLSKDLGLPGFRVGAIYSSNAGVVSAATKMSSFGLVSSQTQHLLASLLGDRDFTRRYIAENTRRIRERREQLAEGLAAVGIECLESNAGLFCWVNMRRLMRSRSFEGEMELWKKVVFEVGLNISPGSSCHCREPGWFRVCFANMSAKTLDVALQRLGAFAEAATAGRRVLAPARSISLPVRFSWANRLTPGSAADRKAER

(19) United States
(12) Patent Application Publication
(10) Pub. No.: US 2013/0326730 A1
(43) Pub. Date: Dec. 5, 2013

(54) DOWN-REGULATION OF ACC SYNTHASE FOR IMPROVED PLANT PERFORMANCE

(71) Applicant: PIONEER HI BRED INTERNATIONAL INC, Johnston, IA (US)

(72) Inventors: Xiaoming Bao, Beijing (CN); Nicholas J. Bate, Raleigh, NC (US); Jeffrey E. Habben, Urbandale, IA (US); Renee Lafitte, Davis, CA (US); Kellie Reimann, Ankeny, IA (US)

(73) Assignee: Pioneer Hi Bred International Inc, Johnston, IA (US)

(21) Appl. No.: 13/867,594

(22) Filed: Apr. 22, 2013

Related U.S. Application Data
(63) Continuation of application No. 12/897,489, filed on Oct. 4, 2010, now abandoned.
(60) Provisional application No. 61/248,060, filed on Oct. 2, 2009.
(60) Provisional application No. 61/290,902, filed on Dec. 30, 2009.
(60) Provisional application No. 61/332,069, filed on May 6, 2010.

(51) Int. Cl. C12N 15/82

(52) U.S. Cl. CPC C12N 15/8249
USPC 800/283; 435/320.1; 435/419; 800/298; 435/412; 435/415; 435/416; 800/320.1; 800/320.3; 800/320.2; 800/320; 800/312; 800/306; 800/322; 800/278

(57) ABSTRACT

Methods for modulating plants using optimized ACC synthase down-regulation constructs are disclosed. Also disclosed are nucleotide sequences, constructs, vectors, and modified plant cells, as well as transgenic plants displaying increased seed and/or biomass yield, improved tolerance to abiotic stress such as drought or high plant density, improved nitrogen utilization efficiency and/or reduction in ethylene production.

### CROSS-REFERENCE

This application is a continuation of U.S. patent application Ser. No. 12/897,489 filed Oct. 4, 2010 and claims priority to U.S. Provisional Patent Application Ser. No. 61/248,060, filed Oct. 2, 2009 and to U.S. Provisional Patent Application Ser. No. 61/290,902, filed Dec. 30, 2009 and to U.S. Provisional Patent Application Ser. No. 61/332,069, filed May 6, 2010, all of which are hereby incorporated by reference herein.

### FIELD OF THE INVENTION

This invention relates generally to the field of molecular biology and the modulation of expression or activity of genes and proteins affecting yield, abiotic stress tolerance and nitrogen utilization efficiency in plants.

### BACKGROUND OF THE INVENTION

Ethylene (C₂H₄) is a gaseous plant hormone. It has a spectrum of effects that can be tissue-specific and/or species-specific. For example, physiological effects include, but are not limited to, promotion of fruit ripening, abscission of leaves and fruit of dicotyledonous species, flower senescence, stem extension of aquatic plants, gas space (aerenchyma) development in roots, leaf epinastic curvature, stem and shoot swelling (often in association with stunting), femaleness in cucurbits, fruit growth in certain species, apical hook closure in etiolated shoots, root hair formation, flowering in the Bromeliaceae, and diageotropism of etiolated shoots. Ethylene is released naturally by ripening fruit and is also produced by most plant tissues, e.g., in response to stress (e.g., density, pathogen attack) and in maturing and senescing organs.

Ethylene is generated from methionine by a biosynthetic pathway involving the conversion of S-adenosyl-L-methionine (SAM or Ado Met) to the cyclic amino acid 1-aminocyclopropane-1-carboxylic acid (ACC) which is facilitated by ACC synthase. Sulphur is conserved in the process by recycling 5′-methylthioadenosine.

ACC synthase is an aminotransferase which catalyzes the rate-limiting step in the formation of ethylene by converting S-adenosylmethionine to ACC. Typically, the enzyme requires pyridoxal phosphate as a cofactor. Features of the invention include ACC synthase sequences and subsequences.

Ethylene is then produced from the oxidation of ACC through the action of ACC oxidase (also known as the ethylene forming enzyme). The ACC oxidase enzyme is stereospecific and uses cofactors, e.g., Fe⁺², O₂, ascorbate, etc. Activity of ACC oxidase can be inhibited by anoxia and cobalt ions. Finally, ethylene can be metabolized by oxidation to CO₂ or to ethylene oxide and ethylene glycol.

The maize ACC synthase (ACS) gene family includes three members: ACS2, ACS6 and ACS7. The identification and analysis of Arabidopsis and tomato ACC synthase mutants deficient in ethylene biosynthesis have helped to establish the important role that ethylene plays in plant growth and development. ACC synthase, the first committed enzyme in the ethylene biosynthetic pathway, plays critical regulatory roles throughout cereal development as well as a key role in regulating responses to environmental stress.

There is a continuing need for modulation of the ethylene production pathway in plants for manipulating plant development or stress responses. This invention relates to the creation of novel ACC synthase downregulation polynucleotide constructs to modulate yield of seed and/or biomass, abiotic stress tolerance, including density tolerance, drought tolerance, nitrogen utilization efficiency and/or ethylene production in plants, including novel polynucleotide sequences, expression cassettes, constructs, vectors, plant cells and resultant plants. These and other features of the invention will become apparent upon review of the following.

### SUMMARY OF THE INVENTION

This invention provides methods and compositions for modulating yield, drought tolerance and/or nitrogen utilization efficiency in plants as well as modulating (e.g., reducing) ethylene production in plants. This invention relates to compositions and methods for down-regulating the level and/or activity of ACC synthase in plants, exemplified by, e.g., SEQ ID NO: 1 and/or SEQ ID NO: 2, including the development of specific RNAi constructs (see, SEQ ID NO: 3) for creation of plants with improved yield and/or improved abiotic stress tolerance, which may include improved drought tolerance, improved density tolerance, and/or improved NUE (nitrogen utilization efficiency). NUE includes both improved yield in low nitrogen conditions and more efficient nitrogen utilization in normal conditions

Therefore, in one aspect, the present invention relates to an isolated nucleic acid comprising a polynucleotide sequence for use in a down-regulation construct, such as an RNAi vector which modulates ACS expression. One embodiment of the invention is an isolated polynucleotide comprising a nucleotide sequence of SEQ ID NO: 1 and/or SEQ ID NO: 2 which may optimize interaction with endogenous RNA sequences.

In another aspect, the present invention relates to recombinant down-regulation constructs comprising the polynucleotides as described (see, SEQ ID NO: 3). The down-regulation constructs generally comprise the polynucleotides of SEQ ID NO: 1 and/or SEQ ID NO: 2 and a promoter operably linked to the same. Additionally, the constructs include several features which result in effective down-regulation of ACS through RNAi embodiments or facilitate modulation of ACS expression. One such feature is the inclusion of one or more FLP/FRT sites. Other features include specific elimination of extraneous open reading frames in the hairpin structure, elimination of an open reading frame from the intron of the ubiquitin promoter, alteration of the hairpin to include an Adhl intron and reconfiguration of the construct so that the hairpin cassette and the herbicide-tolerance marker are in tandem orientation. The invention also relates to a vector containing the recombinant expression cassette. Further, the vector containing the recombinant expression cassette can facilitate the transcription of the nucleic acid in a host cell. The present invention also relates to the host cells able to transcribe a polynucleotide.

In certain embodiments, the present invention is directed to a transgenic plant or plant cell containing a polynucleotide comprising a down-regulation construct. In certain embodiments, a plant cell of the invention is from a dicot or monocot. Preferred plants containing the polynucleotides include, but are not limited to, maize, soybean, sunflower, sorghum, canola, wheat, alfalfa, cotton, rice, barley, tomato and millet. In certain embodiments, the transgenic plant is a maize plant or plant cell. A transgenic seed comprising a transgenic down-regulation construct as described herein is an embodiment. In one embodiment, the plant cell is in a hybrid plant comprising a drought tolerance phenotype and/or a nitrogen utilization efficiency phenotype and/or an improved yield phenotype. In another embodiment, the plant cell is in a plant comprising a sterility phenotype, e.g., a male sterility phenotype. Plants may comprise a combination of such phenotypes. A plant regenerated from a plant cell of the invention is also a feature of the invention.

Certain embodiments have improved drought tolerance as compared to a control plant. The improved drought tolerance of a plant of the invention may reflect physiological aspects such as, but not limited to, (a) a reduction in the production of at least one ACC-synthase-encoding mRNA; (b) a reduction in the production of an ACC synthase; (c) a reduction in the production of ACC; (d) a reduction in the production of ethylene; (e) an increase in plant height or (f) any combination of (a)-(e), compared to a corresponding control plant. Plants exhibiting improved drought tolerance may also exhibit one or more additional abiotic stress tolerance phenotyopes, such as improved nitrogen utilization efficiency and increased density tolerance.

The invention also provides methods for inhibiting ethylene production in a plant and plants produced by such methods. For example, a method of inhibiting ethylene production comprises inhibiting the expression of one or more ACC synthase genes in the plant, wherein the one or more ACC synthase genes encode one or more ACC synthases. Multiple methods and/or multiple constructs may be used to down regulate a single ACC synthase polynucleotide or polypeptide. Multiple ACC synthase polynucleotides or polypeptides may be down-regulated by a single method or by multiple methods; in either case, one or more compositions may be employed.

Methods for modulating drought tolerance in plants are also a feature of the invention, as are plants produced by such methods. For example, a method of modulating drought tolerance comprises: (a) selecting at least one ACC synthase gene (e.g., ACS6) to impact, thereby providing at least one desired ACC synthase gene; (b) introducing a mutant form (e.g., an antisense or sense configuration of at least one ACC synthase gene or subsequence thereof, an RNA silencing configuration of at least one ACC synthase gene or subsequence thereof, and the like) of the at least one desired ACC synthase gene into the plant and (c) expressing the mutant form, thereby modulating drought tolerance in the plant. In certain embodiments, the mutant gene is introduced by Agrobacterium-mediated transfer, electroporation, micro-projectile bombardment, a sexual cross or the like.

Detection of expression products is performed either qualitatively (by detecting presence or absence of one or more product of interest) or quantitatively (by monitoring the level of expression of one or more product of interest). In one embodiment, the expression product is an RNA expression product. Aspects of the invention optionally include monitoring an expression level of a nucleic acid, polypeptide or chemical (e.g., ACC, ethylene, etc.) as noted herein for detection of ACC synthase, ethylene production, drought tolerance, etc., in a plant or in a population of plants.

Kits which incorporate one or more of the nucleic acids noted above are also a feature of the invention. Such kits can include any of the above noted components and further include, e.g., instructions for use of the components in any of the methods noted herein, packaging materials and/or containers for holding the components. For example, a kit for detection of ACS expression levels in a plant includes at least one polynucleotide sequence comprising a nucleic acid sequence, where the nucleic acid sequence is, e.g., at least about 70%, at least about 75%, at least about 80%, at least about 85%, at least about 90%, at least about 95%, at least about 99%, about 99.5% or more, identical to SEQ ID NO: 3 or a subsequence thereof or a complement thereof. The subsequence may be SEQ ID No. 1 or 2. In a further embodiment, the kit includes instructional materials for the use of the at least one polynucleotide sequence to modulate drought tolerance in a plant.

### BRIEF DESCRIPTION OF THE DRAWINGS

FIG. 1 provides details of a plasmid (SEQ ID NO: 3) comprising a hairpin construct. Full sequence of the described plasmid is provided in SEQ ID NO: 3. A ubiquitin promoter (UBI1ZM PRO) drives expression of the hairpin, which comprises TR3 and TR4 (SEQ ID NOS: 1 and 2).

FIG. 2 shows the yield of transformed plants under flowering stress in Season 1, Environment 1. Each bar represents a separate transformation event. Average yield of transgene-negative segregants is shown (139 bu/a) as control (CN). A total of 74% of the events yielded nominally more than the control plants. Plants representing 18 transgenic events outyielded the control at P<0.10.

FIG. 3 shows the yield of transformed plants of the invention under grain-fill stress in Season 1, Environment 2. Each bar represents a separate transformation event. Average yield of transgene-negative segregants is shown (176 bu/a) as control (CN). Thirteen events out-yielded the CN at P<0.10. Of these, eight had also shown significant improvement under flowering stress.

FIG. 4 shows the yield, as a percent of control, of transformed plants of the invention (indicated by a circle), as well as plants transformed using an alternative ACS6 down-regulation vector (indicated by a square) under grain fill stress in Season 1, Environment 3. Each data point represents a separate transformation event. NS=not statistically significant. SIG=statistically significant. The control plants are bulked transgene-negative segregants. As can be seen, 64% of the events of the invention had significantly superior yield; only 17% of the alternative ACS6 down-regulation events had significantly superior yield, relative to the control.

FIG. 5 shows the yield, as a percent of control, of transformed plants of the invention (indicated by a circle), as well as plants transformed using an alternative ACS6 down-regulation vector (indicated by a square) under rain-fed conditions in Season 1, Environment 4. Each data point represents a separate transformation event. NS=not significant. The control plants are bulked transgene-negative segregants. As can be seen, all points exhibiting statistically significant increases in yield represent events of the invention disclosed herein. In addition, all points exhibiting statistically significant decreases in yield are events containing the alternative ACS6 down-regulation vector.

FIG. 6 is a schematic of a representative expression cassette of the invention.

FIG. 7 is an alignment of rice ACS6 coding sequence with the TR4 hairpin truncation (SEQ ID NO: 2).

FIG. 8 is an alignment of maize ACS6 and rice ACS6 sequences.

FIG. 9 shows grain yield (bushels/acre) of events in Background 1 in Season 2.

FIG. 10 shows grain yield (bushels/acre) of events in Background 1 in Season 3.

FIG. 11 shows plant height (inches) of events in Background 1 in Season 3.

FIG. 12 shows grain yield (bushels/acre) and plant height (in inches) of events in Backgrounds 2 and 3 in Season 3.

FIG. 13 shows grain yield (bushels/acre) of events in Backgrounds 2 and 3 across three water treatments and four testers in Season 4.

FIG. 14 provides an alignment of amino acid sequences of ZmACS6 and ZmACS3.

FIG. 15 provides an alignment of coding sequences of ZmACS6 and ZmACS3.

FIG. 16 provides an alignment of TR3 (SEQ ID NO: 1) with ACS3.

FIG. 17 shows ACC levels for four events in transgenic and control root tissues at maize growth stage VT.

FIG. 18 provides quantitative rtPCR data indicating reduced expression of ACS6 in root tissue of maize seedlings transgenic for one of ten ACS downregulation events.

### DETAILED DESCRIPTION OF THE INVENTION

### Definitions

It is to be understood that the terminology used herein is for the purpose of describing particular embodiments only and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

Unless defined otherwise, all technical and scientific terms used herein have the same meaning as commonly understood by one of ordinary skill in the art to which the invention pertains. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present invention now will be described more fully hereinafter with reference to the accompanying drawings and other illustrative non-limiting embodiments.

Many modifications and other embodiments of the invention set forth herein are within the scope of the claimed invention based on the benefit of the teachings in the present descriptions and the associated drawings. Therefore, it is to be understood that the invention is not to be limited to the specific embodiments disclosed and that modifications and other embodiments are intended to be included within the scope of the appended claims.

The practice of the present invention will employ, unless otherwise indicated, conventional techniques of agronomy, botany, microbiology, tissue culture, molecular biology, chemistry, biochemistry and recombinant DNA technology, which are within the skill of the art.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein either by their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32)) can be modified to yield a functionally identical molecule. Accordingly, each silent variation of a nucleic acid is implicit in each described polypeptide sequence and incorporated herein by reference.

As to amino acid sequences, one of skill will recognize that individual substitution, deletion or addition to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, for example, any number of amino acid residues selected from the group of integers consisting of from 1 to 15, such as 1, 2, 3, 4, 5, 7 or 10, can be so altered. Conservatively modified variants typically provide biological activity similar to that of the unmodified polypeptide sequence from which they are derived. For example, substrate specificity, enzyme activity or ligand/receptor binding is generally at least 30%, 40%, 50%, 60%, 70%, 80% or 90%, preferably 60-90% of the binding of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as does the original object polynucleotide and where the hybridization conditions include a wash step in 0.1×SSC and 0.1% sodium dodecyl sulfate at 65° C. Generally, additional sequence or sequences do not materially affect the basic and novel characteristics of the claimed invention, e.g. down-regulation of ACS6. For example, in an embodiment, additional sequences may be included at the 5′ or 3′ end of the hairpin structure without materially affecting the RNA interference function of the construct.

The term “construct” is used to refer generally to an artificial combination of polynucleotide sequences, i.e. a combination which does not occur in nature, normally comprising one or more regulatory elements and one or more coding sequences. The term may include reference to expression cassettes and/or vector sequences, as is appropriate for the context.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control plant may also be a plant transformed with an alternative ACS6 down-regulation construct.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host in which the nucleic acid is to be expressed. For example, although nucleic acid sequences may be expressed in both monocotyledonous and dicotyledonous plant species, sequences can be modified to account for the specific codon preferences and GC content preferences of monocotyledonous plants or dicotyledonous plants (see Murray, et al., (1989) Nucleic Acids Res. 17:477-98, and herein incorporated by reference). Thus, the maize preferred codon for a particular amino acid might be derived from known gene sequences from maize. Maize codon usage for 28 genes from maize plants is listed in Table 4 of Murray, et al., supra.

By “flowering stress” is meant that water is withheld from plants such that drought stress occurs at or around the time of anthesis.

By “grain fill stress” is meant that water is withheld from plants such that drought stress occurs during the time when seeds are accumulating storage products (carbohydrates, protein and/or oil).

By “rain-fed conditions” is meant that water is neither deliberately withheld nor artificially supplemented.

By “well-watered conditions” is meant that water available to the plant is generally adequate for optimum growth.

Drought stress conditions for maize may be controlled to result in a targeted yield reduction. For example, a 20%, 30%, 40%, 50%, 60%, 70% or greater reduction in yield of control plants can be accomplished by providing measured amounts of water during specific phases of plant development.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell which comprises a heterologous nucleic acid sequence of the invention. Host cells may be prokaryotic cells such as E. coli or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet, sugarcane, turfgrass and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “down-regulate” and its forms, e.g. down-regulation, refers to a reduction which may be partial or complete. For example, down-regulation of an ACS polynucleotide in a plant or cell encompasses a reduction in expression to a level that is 99%, 95%, 90%, 85%, 80%, 75%, 70%, 65%, 60%, 55%, 50%, 45%, 40%, 35%, 30%, 25%, 20%, 15%, 10%, 5% or 0% of the expression level of the corresponding ACS polynucleotide in a control plant or cell.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The term “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids which are “isolated”, as defined herein, are also referred to as “heterologous” nucleic acids.

As used herein the term “modulation of ACC synthase activity” shall be interpreted to mean any change in an ACC synthase biological activity, which can include an altered level of ACC synthase present in a plant cell, altered efficacy of the enzyme or any other means which affects one or more of the biological properties of ACC synthase in relation to its role in converting S-adenosylmethionine to ACC in the formation of ethylene. Accordingly, “inhibition of ACC synthase activity” encompasses a reduction in the efficacy of the enzyme or a reduction in the level of ACC synthase present in a plant cell, for example, due to a reduction in the expression of an ACC synthase gene.

In other embodiments, expression of a downregulation construct described herein could modulate other steps along the ethylene synthesis pathway to improve plant yield or abiotic stress tolerance of a plant. For example, the rate of conversion of SAM to polyamines could be increased or the level or activity of ACC oxidase could be decreased or the level or activity of SAM could be increased or some combination of these and/or other modifications in the ethylene synthesis pathway could occur as a result of the genetic modulation described herein. While not wishing to be bound by any theory, it is postulated that modification of one or more steps towards ethylene synthesis results in decreased ethylene activity. In any event, the invention is directed to increasing plant yield in optimum conditions, as well as improving performance under abiotic stress conditions, by modulating expression of an ACC synthase gene, regardless of the precise effect of that modulation on the ethylene synthesis pathway, ethylene production or ethylene activity.

The term “nitrogen utilization efficiency” (NUE) refers to physiological processes of uptake and/or assimilation of nitrogen and/or the subsequent remobilization and reutilization of accumulated nitrogen reserves. Improved NUE refers to enhancement of these processes relative to a control plant. Plants in which NUE is improved may be more productive than control plants under comparable conditions of ample nitrogen availability and/or may maintain productivity under significantly reduced nitrogen availability. Improving NUE, particularly in maize, would increase harvestable yield per unit of input nitrogen fertilizer, both in developing nations where access to nitrogen fertilizer is limited and in developed nations where the level of nitrogen use is high. Improved NUE reduces on-farm input costs, decreases dependence on the non-renewable energy sources required for nitrogen fertilizer production and diminishes the environmental impact of nitrogen fertilizer manufacturing and agricultural use. Improved NUE may be reflected in one or more attributes such as increased biomass, increased grain yield, increased harvest index, increased photosynthetic rates and increased tolerance to biotic or abiotic stress. These attributes may reflect or result in changes including a modulation of root development, shoot and leaf development and/or reproductive tissue development. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion. Furthermore, higher root biomass production may affect production of compounds synthesized by root cells or transgenic root cells or cell cultures of said transgenic root cells. Methods of measuring developmental alterations in the root system are known in the art. See, for example, US Patent Application Publication Number 2003/0074698 and Werner, et al., (2001) PNAS 18:10487-10492, both of which are herein incorporated by reference.

Reducing activity of at least one ACC synthase in a plant can improve the nitrogen stress tolerance of the plant. Such plants may exhibit maintenance of productivity with significantly less nitrogen fertilizer input and/or exhibit enhanced uptake and assimilation of nitrogen fertilizer and/or exhibit altered remobilization and reutilization of accumulated nitrogen reserves or exhibit any combination of such characteristics. In addition to an overall increase in yield, the improvement of nitrogen stress tolerance through the inhibition of ACC synthase can also result in increased root mass and/or length, increased ear, leaf, seed and/or endosperm size and/or improved standability. Accordingly, in some embodiments, the methods further comprise growing said plants under nitrogen limiting conditions and optionally selecting those plants exhibiting greater tolerance to the low nitrogen levels.

Further, methods and compositions are provided for improving yield under abiotic stress, which include evaluating the environmental conditions of an area of cultivation for abiotic stressors (e.g., low nitrogen levels in the soil) and growing plants having reduced ethylene synthesis, which in some embodiments is due to reduced activity of at least one ACC synthase, in stressful environments.

The term “low nitrogen conditions” or “nitrogen limiting conditions” as used herein shall be interpreted to mean any environmental condition in which plant-available nitrogen is less than would be optimal for expression of maximum yield potential.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3 and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, cells in or from seeds, suspension cultures, embryos, meristematic regions, callus tissue, leaves, roots, shoots, gametophytes, sporophytes, pollen and microspores. The class of plants which can be used in the methods of the invention is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (typically 15% for maize, for example) and/or the volume of biomass generated (e.g. for forage crops such as alfalfa, maize for silage and any species grown for biofuel production). Biomass is measured as the weight of harvestable plant material generated.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as do the naturally occurring polynucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and/or other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such as Agrobacterium or Rhizobium.

The term “ACS polypeptide” refers to one or more amino acid sequences of an ACS enzyme. The term is also inclusive of fragments, variants, homologs, alleles or precursors (e.g., preproproteins or proproteins) thereof. An “ACS protein” comprises an ACS polypeptide.

As used herein “recombinant” includes reference to a cell or vector that has been modified by the introduction of a heterologous nucleic acid or a cell that is derived from a cell so modified and maintains the modification. Thus, for example, recombinant cells express genes that are not found in identical form within the native (non-recombinant) form of the cell or express native genes that are otherwise abnormally expressed, under expressed or not expressed at all, as a result of deliberate human intervention or may have reduced or eliminated expression of a native gene. In certain examples, recombinant cells exhibit reduced expression of one or more targeted genes or a reduced level or activity of a polypeptide of interest, relative to the non-recombinant cell. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “residue” and “amino acid residue” and “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, often 60-90% sequence identity and may have 100% sequence identity (i.e., are complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993) and Current Protocols in Molecular Biology, chapter 2, Ausubel, et al., eds, Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, a segment of a full-length cDNA or gene sequence or the complete cDNA or gene sequence.

As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or more nucleotides. Those of skill in the art understand that to avoid inference of inappropriately high similarity to a reference sequence, a gap penalty is typically introduced and is subtracted from the number of matches.

Methods of alignment of nucleotide and amino acid sequences for comparison are well known in the art, such as the local homology algorithm (BESTFIT) of Smith and Waterman, (1981) Adv. Appl. Math 2:482, which may conduct optimal alignment of sequences for comparison; the homology alignment algorithm (GAP) of Needleman and Wunsch, (1970) J. Mol. Biol. 48:443-53; the search for similarity method (Tfasta and Fasta) of Pearson and Lipman, (1988) Proc. Natl. Acad. Sci. USA 85:2444 and computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package®, Version 8 (available from Genetics Computer Group (GCG® programs, Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, such as at least 50% 60%, 70%, 80%, 90% or 95% sequence identity, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, such as 55%, 60%, 70%, 80%, 90% or 95%.

Another indication that nucleotide sequences are substantially identical is that two molecules hybridize to each other under stringent conditions. The degeneracy of the genetic code allows for many nucleic acid substitutions that lead to variety in the nucleotide sequence that code for the same amino acid, hence it is possible that two DNA sequences could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

The terms “substantial identity” in the context of a peptide indicates that a peptide comprises a sequence with between 55-100% sequence identity to a reference sequence, such as 55%, 60%, 70%, 80%, 85%, 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides which are “substantially similar” share sequences as noted above except that residue positions which are not identical may differ by conservative amino acid changes.

### Construction of Nucleic Acids

The isolated nucleic acids can be made using: (a) standard recombinant methods, (b) synthetic techniques or (c) combinations thereof. In some embodiments, the polynucleotides will be cloned, amplified or otherwise constructed from plants, fungi or bacteria.

A nucleic acid, excluding the polynucleotide sequence, is optionally a vector, adapter or linker for cloning and/or expression of a polynucleotide. Additional sequences may be added to such cloning and/or expression sequences to optimize their function in cloning and/or expression, to aid in isolation of the polynucleotide or to improve the introduction of the polynucleotide into a cell. For example one may use recombination sites, such as FRT sites, for creation and isolation of the polynucleotides of the invention, as disclosed in US Patent Application Publication Number 2008/0202505. Examples of recombination sites are known in the art and include FRT sites (See, for example, Schlake and Bode, (1994) Biochemistry 33:12746-12751; Huang, et al., (1991) Nucleic Acids Research 19:443-448; Sadowski, (1995) In Progress in Nucleic Acid Research and Molecular Biology vol. 51, pp. 53-91; Cox, (1989) In Mobile DNA, Berg and Howe, (eds) American Society of Microbiology, Washington D.C., pp. 116-670; Umlauf and Cox, (1988) The EMBO Journal 7:1845-1852; Buchholz, et al., (1996) Nucleic Acids Research 24:3118-3119; Kilby, et al., (1993) Trends Genet. 9:413-421; Rossant and Geagy, (1995) Nat. Med. 1:592-594; Albert, et al., (1995) The Plant Journal 7:649-659; Bayley, et al., (1992) Plant Mol. Biol. 18:353-361; Odell, et al., (1990) Mol. Gen. Genet. 223:369-378 and Dale and Ow, (1991) Proc. Natl. Acad. Sci. USA 88:10558-105620, all of which are herein incorporated by reference.); Lox (Albert, et al., (1995) Plant J. 7:649-659; Qui, et al., (1994) Proc. Natl. Acad. Sci. USA 91:1706-1710; Stuurman, et al., (1996) Plant Mol. Biol. 32:901-913; Odell, et al., (1990) Mol. Gen. Gevet. 223:369-378; Dale, et al., (1990) Gene 91:79-85 and Bayley, et al., (1992) Plant Mol. Biol. 18:353-361; Vega, et al., (2008) Plant Mol. Biol. 66(6):587-598).

Site-specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. Engineering FLP/FRT sites within, or adjacent to, the hairpin structure may facilitate excision of selectable markers and other vector backbone sequence from a host cell.

Use of cloning vectors, expression vectors, adapters and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox and lambda MOSEIox. Optional vectors for the present invention, include but are not limited to, lambda ZAP II and pGEX. For a description of various nucleic acids see, e.g., Stratagene Cloning Systems, Catalogs 1995, 1996, 1997 (La Jolla, Calif.) and Amersham Life Sciences, Inc, Catalog '97 (Arlington Heights, Ill.).

### Synthetic Methods for Constructing Nucleic Acids

The isolated nucleic acids can also be prepared by direct chemical synthesis as known in the art. Chemical synthesis generally produces a single stranded oligonucleotide. This may be converted into double stranded DNA by hybridization with a complementary sequence or by polymerization with a DNA polymerase using the single strand as a template. Longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G> 7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). The number of polynucleotides (3 nucleotides per amino acid) that can be used to determine a codon usage frequency can be any integer from 3 to the number of polynucleotides tested. Optionally, the polynucleotides will be full-length sequences. An exemplary number of sequences for statistical analysis can be at least 1, 5, 10, 20, 50 or 100.

### Recombinant Expression Cassettes

The present invention further provides recombinant expression cassettes comprising a nucleic acid. A recombinant expression cassette will typically comprise a polynucleotide operably linked to transcriptional initiation regulatory sequences which will direct the transcription of the polynucleotide in the intended host cell, such as tissues of a transformed plant.

For example, plant expression vectors may include: (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated or cell- or tissue-specific/preferred expression), a transcription initiation start site, a ribosome binding site, an RNA processing signal, a transcription termination site and/or a polyadenylation signal.

A plant promoter fragment can be employed which will direct expression of a polynucleotide in all, or nearly all, tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill in the art.

Tissue preferred, cell type preferred, developmentally regulated and inducible promoters are examples of “non-constitutive” promoters.

Tissue-preferred promoters can be utilized to target expression within a particular plant tissue. By “tissue-preferred” is intended to mean that expression is predominantly in a particular tissue, albeit not necessarily exclusively in that tissue. Examples include promoters that preferentially initiate transcription in leaves, roots, seeds, endosperm, fibers, xylem vessels, tracheids or sclerenchyma. Certain tissue-preferred promoters may drive expression only in photosynthetic (“green”) tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen Genet. 255(3):337-353; Russell, et al., (1997) Transgenic Res. 6(2):157-168; Rinehart, et al., (1996) Plant Physiol. 112(3):1331-1351; Van Camp, et al., (1996) Plant Physiol. 112(2):525-535; Canevascini, et al., (1996) Plant Physiol. 112(2):513-525; Yamamoto, et al., (1995) Plant Cell Physiol. 35(5):773-778; Lam, (1995) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590; the maize glb1 promoter (GenBank L22344) and Guevara-Garcia, et al., (1993) Plant J. 5(3):595-505. Such promoters can be modified, if necessary, for weak expression. See, also, US Patent Application Number 2003/0074698, herein incorporated by reference.

Shoot-preferred promoters include, shoot meristem-preferred promoters such as promoters disclosed in Weigal, et al., (1992) Cell 69:853-859; Accession Number AJ131822; Accession Number Z71981; Accession Number AF059870, the ZAP promoter (U.S. patent application Ser. No. 10/387,937), the maize tb1 promoter (Wang, et al., (1999) Nature 398:236-239 and shoot-preferred promoters disclosed in McAvoy, et al., (2003) Acta Hort. (ISHS) 625:379-385.

Root-preferred promoters are known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 15(3):533-553 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-651; Leach and Aoyagi, (1991) Plant Science (Limerick) 79(1):69-76); Teeri, et al., (1989) EMBO J. 8(2):353-350. Additional root-preferred promoters include the VfENOD-GRP3 gene promoter (Kuster, et al., (1995) Plant Mol. Biol. 29(5):759-772); rolB promoter (Capana, et al., (1995) Plant Mol. Biol. 25(5):681-691 and the CRWAQ81 root-preferred promoter with the ADH first intron (U.S. Pat. No. 7,411,112). See also, U.S. Pat. Nos. 5,837,876; 5,750,386; 5,633,363; 5,559,252; 5,501,836; 5,110,732 and 5,023,179.

A “cell type”-specific or cell type-preferred promoter primarily drives expression in certain cell types in one or more organs, for example, vascular cells in roots or leaves or mesophyll cells. A mesophyllic cell preferred promoter includes, but is not limited to, known phosphoenopyruvate decarboxylase (PEPC) promoters or putative PEPC promoters from any number of species, for example, Zea mays, Oryza sativa, Arabidopsis thaliana, Glycine max or Sorghum bicolor. Examples include Zea mays PEPC of GenBank Accession Number gi:116268332_HTG AC190686 and gCAT GSS composite sequence; Oryza sativa PEPC of GenBank Accession Number gi|20804452|dbj|AP003052.3|; Arabidopsis thaliana PEPC of GenBank Accession Number gi|5541653|dbj|AP000370.1|AP000370; gi:7769847 or gi|20198070|gb|AC007087.7; Glycine max (GSS contigs) or Sorghum bicolor (JGI assembly scaffold_{—}832, 89230 bp., JGI assembly scaffold_{—}1632, (1997) Plant J. 12(2):255-265; Kwon, et al., (1995) Plant Physiol. 105:357-67; Yamamoto, et al., (1995) Plant Cell Physiol. 35(5):773-778; Gotor, et al., (1993) Plant J. 3:509-18; Orozco, et al., (1993) Plant Mol. Biol. 23(6):1129-1138; Baszczynski, et al., (1988) Nucl. Acid Res. 16:5732; Mitra, et al., (1995) Plant Molecular Biology 26:35-93; Kayaya, et al., (1995) Molecular and General Genetics 258:668-675 and Matsuoka, et al., (1993) Proc. Natl. Acad. Sci. USA 90(20):9586-9590.

The plant promoter may be under more precise environmental control, e.g. the promoter may initiate transcription of an operably-linked gene in response to an external stimulus. Such promoters are referred to here as “inducible” promoters. Environmental conditions that may effect transcription by inducible promoters include pathogen attack, anaerobic conditions or the presence of light. Examples of inducible promoters are the Adh1 promoter, which is inducible by hypoxia or cold stress; the Hsp70 promoter, which is inducible by heat stress; the PPDK promoter, which is inducible by light and abiotic-stress-inducible promoters rab17 (Vilardell, et al., (1991) Plant Mol. Biol. 17(5):985-993); rd29a (Yamaguchi-Shinozaki, et al., (1993) Mol. Gen. Genet. 236:331-340) and KT250 (US Patent Publication Number 2009/0229014); see also, US Patent Publication Number 2004/0123347.

A developmentally regulated promoter may have both a temporal and a spatial limitation, for example, a promoter that drives expression in specific tissue types during pollen development or during inflorescence development. See, e.g., US Patent Publication Numbers 2007/0234444 and 2009/0094713. Another example is a senescence regulated promoter, such as SAM22 (Crowell, et al., (1992) Plant Mol. Biol. 18:559-566); see also, U.S. Pat. No. 5,589,052.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, a polyadenylation region is often included at the 3′-end of a polynucleotide coding region. The polyadenylation region can be derived from a variety of plant genes or from T-DNA. The sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence or the partial coding sequence to increase the amount of the mature message that accumulates in the cytosol; for example, the maize Adh1 and Bz1 introns (Callis, et al., (1987) Genes Dev. 1:1183-1200). Inclusion of a spliceable intron in the transcription unit in expression constructs has been shown to increase gene expression at both the mRNA and protein levels (if applicable) up to 1000-fold (Buchman and Berg, (1988) Mol. Cell Biol. 8:4395-4405). Such intron enhancement of gene expression is typically greatest when placed near the 5′ end of the transcription unit. For a review, see Simpson and Filipowicz, (1996) Plant Mol. Biol. 32:1-41.

Plant signal sequences include, but are not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or barley alpha amylase (BAA) (Rahmatullah, et al., (1989) Plant Mol. Biol. 12:119) or signal peptides which target proteins to the plastids such as that of rapeseed enoyl-Acp reductase (Verwaert, et al., (1994) Plant Mol. Biol. 26:189-202).

A vector comprising the sequences of a polynucleotide of the present invention will typically comprise a marker gene which confers a selectable phenotype on plant cells. The selectable marker gene may encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance. Also useful are genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta and the ALS gene encodes resistance to the herbicide chlorsulfuron. Also useful are genes encoding resistance to glyphosate; see, for example, U.S. Pat. Nos. 7,462,481; 7,531,339; 7,405,075; 7,666,644; 7,622,641 and 7,714,188. Typical vectors useful for expression of genes in higher plants are well known in the art and include vectors derived from the tumor-inducing (Ti) plasmid of Agrobacterium tumefaciens described by Rogers, et al., (1987), Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2, available from CLONTECH Laboratories, Inc. (Palo Alto, Calif.).

### Expression of Sequences in Host Cells

One may express a polynucleotide in a recombinantly engineered cell such as bacteria, yeast, insect or preferably plant cell. The cell produces the polynucleotide in a non-natural condition (e.g., altered in quantity, composition, location and/or time), because it has been genetically altered through human intervention to do so.

It is expected that those of skill in the art are knowledgeable in the numerous expression systems available for expression of a polynucleotide. No attempt will be made to describe in detail all the various methods known for expression in prokaryotes or eukaryotes.

In brief summary, the expression of isolated polynucleotides will typically be achieved by operably linking, for example, the DNA or cDNA to a promoter, followed by incorporation into an expression vector. The vector can be suitable for replication and integration in either prokaryotes or eukaryotes. Typical expression vectors contain transcription and translation terminators, initiation sequences and promoters useful for regulation of the expression of the DNA. To obtain high level expression of a cloned gene, it is desirable to construct expression vectors which contain, at the minimum, a promoter such as ubiquitin to direct transcription, a ribosome binding site for translational initiation and a transcription/translation terminator. Constitutive promoters are classified as providing for a range of constitutive expression. Thus, some are weak constitutive promoters and others are strong constitutive promoters. See, for example, U.S. Pat. No. 6,504,083. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level” or about 1/10 transcripts to about 1/100 transcripts to about 1/1,000 transcripts.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein are available using Bacillus sp. and Salmonella (Palva, et al., (1983) Gene 22:229-35; Mosbach, et al., (1983) Nature 302:543-5).

### Expression in Eukaryotes

A variety of eukaryotic expression systems such as yeast, insect cell lines, plant and mammalian cells are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins can also be ligated to various expression vectors for use in transfecting cell cultures of, for instance, insect or plant origin. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins are available, for instance, from the American Type Culture Collection, P.O. Box 1549, Manassas, Va., USA, 20108.

As with yeast, when plant host cells are employed, polyadenlyation or transcription terminator sequences are typically incorporated into the vector. An example of a terminator sequence is the potato pinll terminator (Keil et al., supra; An et al., supra). Sequences for accurate splicing of the transcript may also be included. An example of a splicing sequence is the VP1 intron from SV40 (Sprague, et al., J. Virol. 45:773-81 (1983)).

### Plant Transformation Methods

Numerous methods for introducing foreign genes into plants are known and can be used to insert an ACS polynucleotide into a plant host, including biological and physical plant transformation protocols. See, e.g., Miki, et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch et al., Science 227:1229-31 (1985)), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, e.g., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722) and ballistic particle acceleration (see, for example, Sanford, et al., U.S. Pat. No. 4,945,050; WO 91/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips, Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209, Longman, N.Y. (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

A widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, a polynucleotide of interest can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the vir gene is present on a separate vector. Such systems, vectors for use therein, and methods of transforming plant cells are described in U.S. Pat. No. 4,658,082; US patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, including but not limited to soybean, maize, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Number 604662 B1 discloses a method for transforming monocots using Agrobacterium. EP Patent Number 672752 B1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Roots or shoots transformed by inoculation of plant tissue with A. rhizogenes or A. tumefaciens can be used as a source of plant tissue to regenerate transgenic plants, either via somatic embryogenesis or organogenesis. Alternatively, plant tissue, in the form of an explant, such as cotyledonary tissue or leaf disks, can be inoculated with these vectors and cultured under conditions which promote plant regeneration. Examples of such methods for regenerating plant tissue are known to those of skill in the art.

### Direct Gene Transfer

Despite the fact that the host range for Agrobacterium-mediated transformation is broad, some major cereal crop species and gymnosperms were initially recalcitrant to this mode of gene transfer. Success and refinements have been reported, both for Agrobacterium-mediated transformation and for alternative methods, collectively referred to as direct gene transfer. For example, with respect to rice, see, Kathuria, et al., (2007) Critical Reviews in Plant Sciences 26:65-103. With respect to wheat, see, He, (2010) J. Exp. Bot 61(6):1567-1581; XiuDao, et al., (2010) Sci. Agri. Sinica 43(8):1539-1553; Zale, (2009) Plant Cell Rep. 28(6):903-913; Wang, et al., (2009) Cereal Res. Commun. 37(1):1-12; Greer, (2009) New Biotech. 26(1/2):44-52. With respect to sugar cane, see, van der Vyver, (2010) Sugar Tech. 12(1):21-25; Joyce, et al., (2010) Plant Cell Rep. 29(2):173-183; Kalunke, et al., (2009) Sugar Tech. 11(4):365-369; Gilbert, et al., (2009) Field Crops Res. 111(1-2):39-46. With respect to turfgrass, see, Cao, (2006) Plant Cell, Tissue, Organ Culture 85(3):307-316.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Reducing the Activity and/or Level of an ACS Polypeptide

Methods are provided to reduce or eliminate the level or activity of an ACS polypeptide by transforming a plant cell with an expression cassette that expresses a polynucleotide that reduces the expression of the ACS polypeptide. The polynucleotide may reduce the expression of the ACS polypeptide directly, by preventing transcription or translation of the ACS messenger RNA, or indirectly, by encoding a polypeptide that reduces the transcription or translation of an ACS gene encoding an ACS polypeptide. Methods for reducing or eliminating the expression of a gene in a plant are well known in the art and any such method may be used in the present invention to reduce the expression of ACS polypeptide.

The expression of an ACS polypeptide is reduced if the level of the ACS polypeptide is less than 100%, 99% 95%, 90%, 85%, 80%, 75%, 70%, 65%, 60%, 55%, 50%, 45%, 40%, 35%, 30%, 25%, 20%, 15%, 10%, 5% or 1% of the level of the same ACS polypeptide in a control plant. In particular embodiments, the level of the ACS polypeptide in a modified plant is less than 60%, less than 50%, less than 40%, less than 30%, less than 20%, less than 10%, less than 5% or less than 2% of the level of the same or a related ACS polypeptide in a control plant. The ACS polynucleotide expression level and/or polypeptide level and/or enzymatic activity may be reduced such that the reduction is phenotypically sufficient to provide tolerance to drought conditions without a yield penalty occurring under well-watered conditions. The level or activity of one or more ACS polynucleotides, polypeptides or enzymes may be impacted. The expression level of the ACS polypeptide may be measured directly, for example, by assaying for the quantity of ACS polypeptide expressed in the plant cell or plant, or indirectly, for example, by measuring the ACS or ethylene synthesis activity in the plant cell or plant or by measuring the phenotypic changes in the plant. Methods for performing such assays are described elsewhere herein.

In certain embodiments of the invention, the activity of the ACS polypeptide is reduced or eliminated by transforming a plant cell with an expression cassette comprising a polynucleotide encoding a polypeptide that inhibits the activity of an ACS polypeptide. The activity of an ACS polypeptide is reduced if the activity of the ACS polypeptide is less than 100%, 99% 95%, 90%, 85%, 80%, 75%, 70%, 65%, 60%, 55%, 50%, 45%, 40%, 35%, 30%, 25%, 20%, 15%, 10%, 5% or 1% of the activity of the same ACS polypeptide in a control plant. In particular embodiments, the ACS activity of the ACS polypeptide in a modified plant is less than 60%, less than 50%, less than 40%, less than 30%, less than 20%, less than 10% or less than 5% of the ACS activity of the same polypeptide in a control plant. The ACS activity of an ACS polypeptide is “eliminated” according to the invention when it is not detectable by the assay methods described elsewhere herein. Methods of determining the alteration of activity of an ACS polypeptide are described elsewhere herein.

In other embodiments, the activity of an ACS polypeptide may be reduced or eliminated by disrupting or excising at least a part of the gene encoding the ACS polypeptide. Mutagenized plants that carry mutations in ACS genes also result in reduced expression of the ACS gene and/or reduced activity of the encoded ACS polypeptide.

Thus, many methods may be used to reduce or eliminate the activity of an ACS polypeptide. One or more methods may be used to reduce the activity of a single ACS polypeptide. One or more methods may be used to reduce the activity of multiple ACS polypeptides.

### 1. Polynucleotide-Based Methods:

In some embodiments, a plant is transformed with an expression cassette that is capable of expressing a polynucleotide that reduces the expression of an ACS polypeptide. The term “expression” as used herein refers to the biosynthesis of a gene product, including the transcription and/or translation of said gene product. For example, an expression cassette capable of expressing a polynucleotide that reduces the expression of at least one ACS polypeptide is an expression cassette capable of producing an RNA molecule that inhibits the transcription and/or translation of at least one ACS polypeptide. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that modulate the expression of an ACS polypeptide are given below.

i. Sense Suppression/Cosuppression

In some embodiments, down-regulation of the expression of an ACS polypeptide may be accomplished by sense suppression or cosuppression. For cosuppression, an expression cassette is designed to express an RNA molecule corresponding to all or part of a messenger RNA encoding an ACS polypeptide in the “sense” orientation. Over-expression of the RNA molecule can result in reduced expression of the native gene. Accordingly, multiple plant lines transformed with the cosuppression expression cassette are screened to identify those that show the reduction of ACS polypeptide expression.

The polynucleotide used for cosuppression may correspond to all or part of the sequence encoding the ACS polypeptide, all or part of the 5′ and/or 3′ untranslated region of an ACS polypeptide transcript or all or part of both the coding sequence and the untranslated regions of a transcript encoding an ACS polypeptide. In some embodiments where the polynucleotide comprises all or part of the coding region for the ACS polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the full-length sequence or a fragment or portion of the transcript of the endogenous gene, generally greater than about 65% sequence identity, often greater than about 85% sequence identity, sometimes greater than about 95% sequence identity. See, U.S. Pat. Nos. 5,283,184 and 5,034,323, herein incorporated by reference.

ii. Antisense Suppression

In some embodiments, reduction of the expression of the ACS polypeptide may be obtained by antisense suppression. For antisense suppression, the expression cassette is designed to express an RNA molecule complementary to all or part of a messenger RNA encoding the ACS polypeptide. Over expression of the antisense RNA molecule can result in reduced expression of the native gene. Accordingly, multiple plant lines transformed with the antisense suppression expression cassette are screened to identify those that show the optimum down-regulation of ACS polypeptide expression.

The polynucleotide for use in antisense suppression may correspond to all or part of the complement of the sequence encoding the ACS polypeptide, all or part of the complement of the 5′ and/or 3′ untranslated region of the ACS transcript or all or part of the complement of both the coding sequence and the untranslated regions of a transcript encoding the ACS polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or more nucleotides may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the invention, down-regulation of the expression of an ACS polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in down-regulation of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the optimum down-regulation of ACS polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 99/49029, WO 99/53050, WO 99/61631 and WO 00/49035, each of which is herein incorporated by reference.

iv. Hairpin RNA Interference and Intron-Containing Hairpin RNA Interference

In some embodiments of the invention, down-regulation of the expression of an ACS polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. The antisense sequence may be located “upstream” of the sense sequence (i.e., the antisense sequence may be closer to the promoter driving expression of the hpRNA than is the sense sequence.) The base-paired stem region may correspond to a portion of a promoter sequence controlling expression of the gene to be inhibited. Thus, the base-paired stem region of the molecule generally determines the specificity of the RNA interference. The sense sequence and the antisense sequence are generally of similar lengths but may differ in length. Thus, these sequences may be portions or fragments of at least 10, 19, 20, 21, 22, 23, 24, 25, 26, 27, 28, 29, 30, 50, 70, 90, 100, 120, 140, 160, 180, 200, 220, 240, 260, 280, 300, 320, 340, 360, 380, 400, 500, 600, 700, 800 or 900 nucleotides in length or at least 1, 2, 3, 4, 5, 6, 7, 8, 9 or 10 kb in length. The loop region of the expression cassette may vary in length. Thus, the loop region may be at least 50, 80, 100, 200, 300, 400, 500, 600, 700, 800 or 900 nucleotides in length or at least 1, 2, 3, 4, 5, 6, 7, 8, 9 or 10 kb in length.

hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to reduce or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing and this increases the efficiency of interference. In some embodiments, the intron is the Adh1 intron 1. Methods for using ihpRNA interference to inhibit the expression of endogenous plant genes are described, for example, in Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant-virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the ACS polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,635,805, each of which is herein incorporated by reference.

vi. Ribozymes

In some embodiments, the polynucleotide expressed by the expression cassette is catalytic RNA or has ribozyme activity specific for the messenger RNA of the ACS polypeptide. Thus, the polynucleotide causes the degradation of the endogenous messenger RNA, resulting in reduced expression of the ACS polypeptide. This method is described, for example, in U.S. Pat. No. 4,987,071, herein incorporated by reference.

### Methods for Modulating Drought Tolerance in a Plant

Methods for modulating drought tolerance in plants are also features of the invention. The ability to introduce different degrees of drought tolerance into plants offers flexibility in the use of the invention: for example, introduction of strong drought tolerance for improved grain-filling or for silage in areas with longer or drier growing seasons, versus the introduction of a moderate drought tolerance for silage in agricultural areas with shorter growing seasons. Modulation of drought tolerance of a plant of the invention may reflect one or more of the following: (a) a reduction in the production of at least one ACC-synthase-encoding mRNA; (b) a reduction in the production of an ACC synthase; (c) a reduction in the production of ACC; (d) a reduction in the production of ethylene; (e) an increase in plant height or (f) any combination of (a)-(e), compared to a corresponding control plant.

For example, a method of the invention can include: (a) selecting at least one ACC synthase gene to mutate, thereby providing at least one desired ACC synthase gene; (b) introducing a mutant form of the at least one desired ACC synthase gene into the plant and (c) expressing the mutant form, thereby modulating drought tolerance in the plant. Plants produced by such methods are also a feature of the invention.

The degree of drought tolerance introduced into a plant can be determined by a number of factors, e.g., which ACC synthase gene is selected, whether the mutant gene member is present in a heterozygous or homozygous state or by the number of members of this family which are inactivated or by a combination of two or more such factors.

Once the desired ACC synthase gene is selected, a mutant form of the ACC synthase gene is introduced into a plant. In certain embodiments, the mutant form is introduced by Agrobacterium-mediated transfer, electroporation, micro-projectile bombardment, homologous recombination or a sexual cross. In certain embodiments, the mutant form includes, e.g., a heterozygous mutation in the at least one ACC synthase gene, a homozygous mutation in the at least one ACC synthase gene or a combination of homozygous mutation and heterozygous mutation if more than one ACC synthase gene is selected. In another embodiment, the mutant form includes a subsequence of the at least one desired ACC synthase gene in an antisense, sense or RNA silencing or interference configuration.

Expression of the mutant form of the ACC synthase gene can be determined in a number of ways. For example, detection of expression products is performed either qualitatively (presence or absence of one or more product of interest) or quantitatively (by monitoring the level of expression of one or more product of interest). In one embodiment, the expression product is an RNA expression product. The invention optionally includes monitoring an expression level of a nucleic acid or polypeptide as noted herein for detection of ACC synthase in a plant or in a population of plants. Monitoring levels of ethylene or ACC can also serve to detect down-regulation of expression or activity of the ACC synthase gene.

### Methods for Modulating Density Tolerance in a Plant

In addition to increasing tolerance to drought stress in plants of the invention compared to a control plant, the invention also enables higher density planting of plants of the invention, leading to increased yield per acre of corn. Most of the increased yield per acre of corn over the last century has come from increasing tolerance to density, which is a stress to plants. Methods for modulating plant stress response, e.g., increasing tolerance for density, are also a feature of the invention. For example, a method of the invention can include: (a) selecting at least one ACC synthase gene to mutate, thereby providing at least one desired ACC synthase gene; (b) introducing a mutant form of the at least one desired ACC synthase gene into the plant and (c) expressing the mutant form, thereby modulating density tolerance in the plant. Plants produced by such methods are also a feature of the invention. When ethylene production is reduced in a plant by a mutant form of a desired ACC synthase gene, the plant may have a reduced perception of and/or response to density. Thus, plants of the invention can be planted at higher density than currently practiced by farmers and produce an increase in yield of seed and/or biomass.

### Methods for Modulating Nitrogen Utilization Efficiency in a Plant

In addition to increasing tolerance to drought stress and improving density stress tolerance in plants of the invention compared to a control plant, the invention also may provide greater nitrogen utilization efficiency. For example, a method of the invention can include: (a) selecting at least one ACC synthase gene to mutate, thereby providing at least one desired ACC synthase gene; (b) introducing a mutant form of the at least one desired ACC synthase gene into the plant and (c) expressing the mutant form, thereby modulating NUE in the plant. Plants produced by such methods are also a feature of the invention. Plants in which NUE is improved may be more productive than control plants under comparable conditions of ample nitrogen availability and/or may maintain productivity under significantly reduced nitrogen availability. Improved NUE may be reflected in one or more attributes such as increased biomass, increased grain yield, increased harvest index, increased photosynthetic rates and increased tolerance to biotic or abiotic stress. In particular, improving NUE in maize would increase harvestable yield per unit of input nitrogen fertilizer, both in developing nations where access to nitrogen fertilizer is limited and in developed nations where the level of nitrogen use remains high.

### Screening/Characterization of Plants or Plant Cells

Plants can be screened and/or characterized genotypically, biochemically, phenotypically or by a combination of two or more of these methods. For example, plants may be characterized to determine the presence, absence and/or expression level (e.g., amount, modulation, such as a decrease or increase compared to a control cell) of a polynucleotide of the invention; the presence, absence, expression and/or enzymatic activity of a polypeptide of the invention and/or modulation of drought tolerance, modulation of nitrogen use efficiency, modulation of density tolerance and/or modulation of ethylene production.

Chemicals, e.g., ethylene, ACC, etc., can be recovered and assayed from the cell extracts. For example, internal concentrations of ACC can be assayed by gas chromatography-mass spectroscopy, in acidic plant extracts as ethylene after decomposition in alkaline hypochlorite solution, etc. The concentration of ethylene can be determined by, e.g., gas chromatography-mass spectroscopy, etc. See, e.g., Nagahama, et al., (1991) J. Gen. Microbiol. 137:2281 2286. For example, ethylene can be measured with a gas chromatograph equipped with, e.g., an alumina based column (such as an HP-PLOT A1203 capillary column (Agilent Technologies, Santa Clara, Calif.) and a flame ionization detector.

Phenotypic analysis includes, e.g., analyzing changes in chemical composition, morphology or physiological properties of the plant. For example, phenotypic changes can include, but are not limited to, an increase in drought tolerance, an increase in density tolerance, an increase in nitrogen use efficiency and a decrease in ethylene production.

A variety of assays can be used for monitoring drought tolerance and/or NUE. For example, assays include, but are not limited to, visual inspection, monitoring photosynthesis measurements and measuring levels of chlorophyll, DNA, RNA and/or protein content of, e.g., the leaves, under stress and non-stress conditions.

### Plants of the Invention

Plant cells useful in the invention include, but are not limited to, meristem cells, Type I, Type II and Type III callus, immature embryos and gametic cells such as microspores, pollen, sperm and egg. In certain embodiments, the plant cell of the invention is from a dicot or monocot. A plant regenerated from the plant cell(s) of the invention is also a feature of the invention.

In one embodiment, the plant cell is in a plant, e.g., a hybrid plant, comprising a drought tolerant phenotype. In another embodiment, the plant cell is in a plant comprising a sterility phenotype, e.g., a male sterility phenotype. Through a series of breeding manipulations, the construct impacting an ACC synthase gene can be moved from one plant line to another plant line. For example, a hybrid plant can be produced by sexual cross of a plant comprising a modified expression of one or more ACC synthase genes and a control plant.

Modified plant cells are also a feature of the invention. In a first aspect, the invention provides for an isolated or recombinant plant cell comprising at least one down-regulation construct capable of inhibiting an endogenous ACC synthase gene; e.g., a nucleic acid sequence, or complement thereof, comprising, e.g., at least about 70%, at least about 75%, at least about 80%, at least about 85%, at least about 90%, at least about 95%, at least about 99%, about 99.5% or more, sequence identity to the ACS6 down-regulation expression construct of SEQ ID NO: 4, SEQ ID NO: 5, SEQ ID NO: 6 or SEQ ID NO: 7. The down-regulation of expression or activity of at least one ACC synthase polynucleotide or protein is compared to a corresponding control plant cell lacking the down-regulation construct. Essentially any plant can be used in the methods and compositions of the invention. Such species include, but are not restricted to, members of the families Poaceae (formerly Graminae), including Zea mays (corn or maize), rye, triticale, barley, millet, rice, wheat, oats, etc.; Leguminosae, including pea, beans, lentil, peanut, yam bean, cowpeas, velvet beans, soybean, clover, alfalfa, lupine, vetch, lotus, sweet clover, wisteria, sweetpea, etc.; Compositae, the largest family of vascular plants, including at least 1,000 genera, including important commercial crops such as sunflower; Rosaciae, including raspberry, apricot, almond, peach, rose, etc.; as well as nut plants, including, walnut, pecan, hazelnut, etc., forest trees (including Pinus, Quercus, Pseutotsuga, Sequoia, Populus, etc. and other common crop plants, e.g., cotton, sorghum, lawn grasses, tomato, potato, pepper, canola, broccoli, cabbage, etc.

Additional plants, as well as those specified above, include plants from the genera: Acamptoclados, Achnatherum, Achnella, Acroceras, Aegilops, Aegopgon, Agroelymus, Agrohordeum, Agropogon, Agropyron, Agrositanion, Agrostis, Aira, Allolepis, Alloteropsis, Alopecurus, Amblyopyrum, Ammophila, Ampelodesmos, Amphibromus, Amphicarpum, Amphilophis, Anastrophus, Anatherum, Andropogron, Anemathele, Aneurolepidium, Anisantha, Anthaenantia, Anthephora, Anthochloa, Anthoxanthum, Apera, Apluda, Archtagrostis, Arctophila, Argillochloa, Aristida, Arrhenatherum, Arthraxon, Arthrostylidium, Arundinaria, Arundinella, Arundo, Aspris, Atheropogon, Avena (e.g., oats), Avenella, Avenochloa, Avenula, Axonopus, Bambusa, Beckmannia, Blepharidachne, Blepharoneuron, Bothriochloa, Bouteloua, Brachiaria, Brachyelytrum, Brachypodium, Briza, Brizopyrum, Bromelica, Bromopsis, Bromus, Buchloe, Bulbilis, Calamagrostis, Calamovilfa, Campulosus, Capriola, Catabrosa, Catapodium, Cathestecum, Cenchropsis, Cenchrus, Centotheca, Ceratochloa, Chaetochloa, Chasmanthium, Chimonobambusa, Chionochloa, Chloris, Chondrosum, Chrysopon, Chusquea, Cinna, Cladoraphis, Coelorachis, Coix, Coleanthus, Colpodium, Coridochloa, Cornucopiae, Cortaderia, Corynephorus, Cottea, Critesion, Crypsis, Ctenium, Cutandia, Cylindropyrum, Cymbopogon, Cynodon, Cynosurus, Cytrococcum, Dactylis, Dactyloctenium, Danthonia, Dasyochloa, Dasyprum, Davyella, Dendrocalamus, Deschampsia, Desmazeria, Deyeuxia, Diarina, Diarrhena, Dichanthelium, Dichanthium, Dichelachne, Diectomus, Digitaria, Dimeria, Dimorpostachys, Dinebra, Diplachne, Dissanthelium, Dissochondrus, Distichlis, Drepanostachyum, Dupoa, Dupontia, Echinochloa, Ectosperma, Ehrharta, Eleusine, Elyhordeum, Elyleymus, Elymordeum, Elymus, Elyonurus, Elysitanion, Elytesion, Elytrigia, Enneapogon, Enteropogon, Epicampes, Eragrostis, Eremochloa, Eremopoa, Eremopyrum, Erianthus, Ericoma, Erichloa, Eriochrysis, Erioneuron, Euchlaena, Euclasta, Eulalia, Eulaliopsis, Eustachys, Fargesia, Festuca, Festulolium, Fingerhuthia, Fluminia, Garnotia, Gastridium, Gaudinia, Gigantochloa, Glyceria, Graphephorum, Gymnopogon, Gynerium, Hackelochloa, Hainardia, Hakonechloa, Haynaldia, Heleochloa, Helictotrichon, Hemarthria, Hesperochloa, Hesperostipa, Heteropogon, Hibanobambusa, Hierochloe, Hilaria, Holcus, Homalocenchrus, Hordeum (e.g., barley), Hydrochloa, Hymenachne, Hyparrhenia, Hypogynium, Hystrix, Ichnanthus, Imperata, Indocalamus, Isachne, lschaemum, Ixophorus, Koeleria, Korycarpus, Lagurus, Lamarckia, Lasiacis, Leersia, Leptochloa, Leptochloopsis, Leptocoryphium, Leptoloma, Leptogon, Lepturus, Lerchenfeldia, Leucopoa, Leymostachys, Leymus, Limnodea, Lithachne, Lolium, Lophochlaena, Lophochloa, Lophopyrum, Ludolfia, Luziola, Lycurus, Lygeum, Maltea, Manisuris, Megastachya, Melica, Melinis, Mibora, Microchloa, Microlaena, Microstegium, Milium, Miscanthus, Mnesithea, Molinia, Monanthochloe, Monerma, Monroa, Muhlenbergia, Nardus, Nassella, Nazia, Neeragrostis, Neoschischkinia, Neostapfia, Neyraudia, Nothoholcus, Olyra, Opizia, Oplismenus, Orcuttia, Oryza (e.g., rice), Oryzopsis, Otatea, Oxytenanthera, Particularia, Panicum, Pappophorum, Parapholis, Pascopyrum, Paspalidium, Paspalum, Pennisetum (e.g., millet), Phalaris, Phalaroides, Phanopyrum, Pharus, Phippsia, Phleum, Pholiurus, Phragmites, Phyllostachys, Piptatherum, Piptochaetium, Pleioblastus, Pleopogon, Pleuraphis, Pleuropogon, Poa, Podagrostis, Polypogon, Polytrias, Psathyrostachys, Pseudelymus, Pseudoroegneria, Pseudosasa, Ptilagrostis, Puccinellia, Pucciphippsia, Redfieldia, Reimaria, Reimarochloa, Rhaphis, Rhombolytrum, Rhynchelytrum, Roegneria, Rostraria, Rottboellia, Rytilix, Saccharum, Sacciolepis, Sasa, Sasaella, Sasamorpha, Savastana, Schedonnardus, Schismus, Schizachne, Schizachyrium, Schizostachyum, Sclerochloa, Scleropoa, Scleropogon, Scolochloa, Scribneria, Secale (e.g., rye), Semiarundinaria, Sesleria, Setaria, Shibataea, Sieglingia, Sinarundinaria, Sinobambusa, Sinocalamus, Sitanion, Sorghastrum, Sorghum, Spartina, Sphenopholis, Spodiopogon, Sporobolus, Stapfia, Steinchisma, Stenotaphrum, Stipa, Stipagrostis, Stiporyzopsis, Swallenia, Syntherisma, Taeniatherum, Terrellia, Terrelymus, Thamnocalamus, Themeda, Thinopyrum, Thuarea, Thysanolaena, Torresia, Torreyochloa, Trachynia, Trachypogon, Tragus, Trichachne, Trichloris, Tricholaena, Trichoneura, Tridens, Triodia, Triplasis, Tripogon, Tripsacum, Trisetobromus, Trisetum, Triticosecale, Triticum (e.g., wheat), Tuctoria, Uniola, Urachne, Uralepis, Urochloa, Vahlodea, Valota, Vaseyochloa, Ventenata, Vetiveria, Vilfa, Vulpia, Willkommia, Yushania, Zea (e.g., corn), Zizania, Zizaniopsis and Zoysia.

### Regeneration of Isolated, Recombinant or Transgenic Plants

Transformed plant cells which are derived by plant transformation techniques and isolated or recombinant plant cells derived therefrom, including those discussed above, can be cultured to regenerate a whole plant which possesses the desired genotype (i.e., comprising an ACC synthase down-regulation nucleic acid) and/or thus the desired phenotype, e.g., improved NUE and/or drought tolerance phenotype, density tolerant phenotype, etc. The desired cells, which can be identified, e.g., by selection or screening, are cultured in medium that supports regeneration. The cells can then be allowed to mature into plants. For example, such regeneration techniques can rely on manipulation of certain phytohormones in a tissue culture growth medium, typically relying on a biocide and/or herbicide marker which has been introduced into the plant together with the desired nucleotide sequences. Alternatively, cells, tissues or plants can be screened for down-regulation of expression and/or activity of ACC synthase, reduction in ethylene production conferred by the ACC synthase down-regulation nucleic acid sequence, etc. Plant regeneration from cultured protoplasts is described in Evans, et al., (1983) Protoplasts Isolation and Culture, Handbook of Plant Cell Culture, pp 124 176, Macmillan Publishing Company, New York; Davey, (1983) Protoplasts, pp. 12-29, Birkhauser, Basal 1983; Dale, (1983) Protoplasts pp. 31-41, Birkhauser, Basel and Binding (1985) Regeneration of Plants, Plant Protoplasts pp 21-73, CRC Press, Boca Raton. Regeneration can also be obtained from plant callus, explants, organs or parts thereof. Such regeneration techniques are described generally in Klee, et al., (1987) Ann Rev of Plant Phys 38:467-486. See also, e.g., Payne and Gamborg. For transformation and regeneration of maize see, for example, U.S. Pat. No. 5,736,369.

Plants cells transformed with a plant expression vector can be regenerated, e.g., from single cells, callus tissue or leaf discs according to standard plant tissue culture techniques. It is well known in the art that various cells, tissues and organs from almost any plant can be successfully cultured to regenerate an entire plant. Plant regeneration from cultured protoplasts is described in Evans, et al., Protoplasts Isolation and Culture, Handbook of Plant Cell Culture, Macmillilan Publishing Company, New York, pp. 124-176 (1983) and Binding, Regeneration of Plants, Plant Protoplasts, CRC Press, Boca Raton, pp. 21-73 (1985).

The regeneration of plants containing the foreign gene introduced by Agrobacterium from leaf explants can be achieved as described by Horsch, et al., (1985) Science 227:1229-1231. After transformation with Agrobacterium, the explants typically are transferred to selection medium. One of skill will realize that the selection medium depends on the selectable marker that is co-transfected into the explants. In this procedure, transformants are grown in the presence of a selection agent and in a medium that induces the regeneration of shoots in the plant species being transformed as described by Fraley, et al., (1983) Proc. Nat'l. Acad. Sci. USA, 80:4803. This procedure typically produces shoots, e.g., within two to four weeks, and these transformant shoots (which are typically about 1-2 cm in length) are then transferred to an appropriate root-inducing medium containing the selective agent and an antibiotic to prevent bacterial growth. Selective pressure is typically maintained in the root and shoot medium.

Typically, the transformants will develop roots in about 1-2 weeks and form plantlets. After the plantlets are about 3-5 cm in height, they are placed in sterile soil in fiber pots. Those of skill in the art will realize that different acclimation procedures are used to obtain transformed plants of different species. For example, after developing a root and shoot, cuttings, as well as somatic embryos of transformed plants, are transferred to medium for establishment of plantlets. For a description of selection and regeneration of transformed plants, see, e.g., Dodds and Roberts, (1995) Experiments in Plant Tissue Culture, 3rd Ed., Cambridge University Press. Transgenic plants may be fertile or sterile.

The regeneration of plants from either single plant protoplasts or various explants is well known in the art. See, for example, Methods for Plant Molecular Biology, Weissbach and Weissbach, eds., Academic Press, Inc., San Diego, Calif. (1988). This regeneration and growth process includes the steps of selection of transformant cells and shoots, rooting the transformant shoots and growth of the plantlets in soil. For maize cell culture and regeneration see generally, The Maize Handbook, Freeling and Walbot, Eds., Springer, N.Y. (1994); Corn and Corn Improvement, 3rd edition, Sprague and Dudley, Eds., American Society of Agronomy, Madison, Wis. (1988).

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed-propagated crops, mature transgenic plants can be self-pollinated to produce a homozygous inbred plant. The inbred plant produces seed containing the newly introduced heterologous nucleic acid. These seeds can be grown to produce plants that would produce the selected phenotype. Mature transgenic plants can also be crossed with other appropriate plants, generally another inbred or hybrid, including, for example, an isogenic untransformed inbred.

Parts obtained from the regenerated plant, such as flowers, seeds, leaves, branches, fruit and the like are included in the invention, provided that these parts comprise cells comprising the down-regulation construct or a functional fragment thereof. Progeny and variants and mutants of the regenerated plants are also included within the scope of the invention, provided that these plants comprise the down-regulation construct or a functional fragment thereof.

Transgenic plants expressing the selectable marker can be screened for transmission of the down-regulation construct by, for example, standard immunoblot and DNA detection techniques. Transgenic lines are also typically evaluated for levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. In addition, in situ hybridization and immunocytochemistry according to standard protocols can be done using heterologous nucleic acid specific polynucleotide probes to localize sites of expression within transgenic tissue. Generally, a number of transgenic lines are screened for the incorporated nucleic acid to identify and select plants with the most appropriate expression profiles.

Some embodiments comprise a transgenic plant that is homozygous for the added heterologous nucleic acid; i.e., a transgenic plant that contains two added nucleic acid sequences at corresponding loci on each chromosome of a chromosome pair. A homozygous transgenic plant can be obtained by sexually mating (selfing) a heterozygous (aka hemizygous) transgenic plant that contains a single added heterologous nucleic acid, germinating some of the seed produced and analyzing the resulting plants produced for altered expression of a polynucleotide of the present invention relative to a control plant. Back-crossing to a parental plant and out-crossing with a non-transgenic plant or with a plant transgenic for the same or another trait or traits are also contemplated.

It is also expected that the transformed plants will be used in traditional breeding programs, including TOPCROSS pollination systems as disclosed in U.S. Pat. No. 5,706,603 and U.S. Pat. No. 5,704,160, the disclosure of each of which is incorporated herein by reference.

In addition to Berger, Ausubel and Sambrook, useful general references for plant cell cloning, culture and regeneration include Jones, (ed) (1995) Plant Gene Transfer and Expression Protocols—Methods in Molecular Biology, Volume 49 Humana Press Towata NJ; Payne, et al., (1992) Plant Cell and Tissue Culture in Liquid Systems, John Wiley & Sons, Inc. New York, N.Y. (Payne) and Gamborg and Phillips, (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks, (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St. Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, UK.

### “Stacking” of Constructs and Traits

In certain embodiments, the nucleic acid sequences of the present invention can be used in combination (“stacked”) with other polynucleotide sequences of interest in order to create plants with a desired phenotype. The polynucleotides of the present invention may be stacked with any gene or combination of genes and the combinations generated can include multiple copies of any one or more of the polynucleotides of interest. Stacking can be performed either through molecular stacking or through a conventional breeding approach. Site-specific integration of one or more transgenes at the ACS locus is also possible. The desired combination may affect one or more traits; that is, certain combinations may be created for modulation of gene expression affecting ACC synthase activity and/or ethylene production. Other combinations may be designed to produce plants with a variety of desired traits, including but not limited to traits desirable for animal feed such as high oil genes (e.g., U.S. Pat. No. 6,232,529); balanced amino acids (e.g. hordothionins (U.S. Pat. Nos. 5,990,389; 5,885,801; 5,885,802 and 5,703,409); barley high lysine (Williamson, et al., (1987) Eur. J. Biochem. 165:99-106 and WO 98/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS and/or glyphosate N-acetyltransferase (GAT) genes; see, for example, U.S. Pat. Nos. 7,462,481; 7,531,339; 7,405,075; 7,666,644; 7,622,641 and 7,714,188); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see, U.S. Pat. No. 5,583,210), stalk strength, flowering time or transformation technology traits such as cell cycle regulation or gene targeting (e.g. WO 99/61619; WO 00/17364; WO 99/25821), the disclosures of which are herein incorporated by reference.

For example, in addition to an ACS downregulation expression cassette (which may be an ACS6 downregulation expression cassette), a stacked combination may include one or more expression cassettes providing one or more of the following: modulation of ABA perception/response targeted to reproductive tissues (e.g., eepl promoter driving Arabidopsis ABI1 mutant; see, US Patent Publication Number 2004/0148654); modulation of cytokinin expression or activity (see, e.g., US Patent Publication Number 2009/0165177 and U.S. Pat. No. 6,992,237); modulation of cis-prenyltransferase expression or activity (see, e.g., U.S. Pat. Nos. 6,645,747 and 7,273,737; modulation of cellulose synthase (see, e.g., U.S. Pat. Nos. 7,214,852 and 7,524,933). In one or more of these stacks, the ACS downregulation expression cassette may comprise a tissue-preferred promoter (see, e.g., the eep5 promoter disclosed in US Patent Publication Number 2009/0307800 or the eepl promoter disclosed in US Patent Publication Number 2004/0237147).

These stacked combinations can be created by any method, including but not limited to cross breeding plants by any conventional or TopCross methodology or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences of interest can be driven by the same promoter or by different promoters. In certain cases, it may be desirable to introduce a transformation cassette that will suppress the expression of a polynucleotide of interest. This may be accompanied by any combination of other suppression cassettes or over-expression cassettes to generate the desired combination of traits in the plant.

### Use in Breeding Methods

The transformed plants of the invention may be used in a plant breeding program. The goal of plant breeding is to combine, in a single variety or hybrid, various desirable traits. For field crops, these traits may include, for example, resistance to diseases and insects, tolerance to heat and drought, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This invention encompasses methods for producing a maize plant by crossing a first parent maize plant with a second parent maize plant wherein one or both of the parent maize plants is a transformed plant displaying a drought tolerance phenotype, a sterility phenotype, a density tolerance phenotype or the like, as described herein.

Plant breeding techniques known in the art and used in a maize plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of maize hybrids in a maize plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular maize plant using transformation techniques can be moved into another line using traditional breeding techniques that are well known in the plant breeding arts. For example, a backcrossing approach is commonly used to move a transgene from a transformed maize plant to an elite inbred line and the resulting progeny would then comprise the transgene(s). Also, if an inbred line was used for the transformation, then the transgenic plants could be crossed to a different inbred in order to produce a transgenic hybrid maize plant. As used herein, “crossing” can refer to a simple X by Y cross or the process of backcrossing, depending on the context.

The development of a maize hybrid in a maize plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process in maize, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Transgenic plants of the present invention may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Kits for Modulating Drought Tolerance or Other Traits

Certain embodiments of the invention can optionally be provided to a user as a kit. For example, a kit of the invention can contain one or more nucleic acid, polypeptide, antibody, diagnostic nucleic acid or polypeptide, e.g., antibody, probe set, e.g., as a cDNA microarray, one or more vector and/or cell line described herein. Most often, the kit is packaged in a suitable container. The kit typically further comprises one or more additional reagents, e.g., substrates, labels, primers or the like for labeling expression products, tubes and/or other accessories, reagents for collecting samples, buffers, hybridization chambers, cover slips, etc. The kit optionally further comprises an instruction set or user manual detailing preferred methods of using the kit components for discovery or application of gene sets. When used according to the instructions, the kit can be used, e.g., for evaluating expression or polymorphisms in a plant sample, e.g., for evaluating ACC synthase activity, ethylene production, density resistance potential, sterility, etc. Alternatively, the kit can be used according to instructions for using at least one ACC synthase polynucleotide sequence to modulate drought tolerance in a plant.

As another example, a kit includes a container containing at least one polynucleotide sequence comprising a nucleic acid sequence, wherein the nucleic acid sequence is, e.g., at least about 70%, at least about 75%, at least about 80%, at least about 85%, at least about 90%, at least about 95%, at least about 99%, about 99.5% or more, identical to SEQ ID NO: 1, 2, 3 or 4 or a subsequence thereof or a complement thereof. The kit optionally also includes instructional materials for the use of the at least one polynucleotide sequence in a plant.

### Other Nucleic Acid and Protein Assays

In the context of the invention, nucleic acids and/or proteins are manipulated according to well known molecular biology methods. Detailed protocols for numerous such procedures are described in, e.g., in Ausubel, et al., Current Protocols in Molecular Biology (supplemented through 2004) John Wiley & Sons, New York (“Ausubel”); Sambrook, et al., Molecular Cloning—A Laboratory Manual (2nd Ed.), Vol. 1-3, Cold Spring Harbor Laboratory, Cold Spring Harbor, N.Y., (1989) (“Sambrook”) and Berger and Kimmel, Guide to Molecular Cloning Techniques, Methods in Enzymology volume 152 Academic Press, Inc., San Diego, Calif. (“Berger”).

In addition to the above references, protocols for in vitro amplification techniques, such as the polymerase chain reaction (PCR), the ligase chain reaction (LCR), Qβ-replicase amplification and other RNA polymerase mediated techniques (e.g., NASBA), useful, e.g., for amplifying polynucleotides of the invention, are found in Mullis, et al., (1987) U.S. Pat. No. 4,683,202; PCR Protocols A Guide to Methods and Applications (Innis, et al., eds) Academic Press Inc. San Diego, Calif. (1990) (“Innis”); Arnheim and Levinson, (1990) C&EN 36; The Journal Of NIH Research (1991) 3:81; Kwoh, et al., (1989) Proc Natl Acad Sci USA 86:1173; Guatelli, et al., (1990) Proc Natl Acad Sci USA 87:1874; Lomell, et al., (1989) J Clin Chem 35:1826; Landegren, et al., (1988) Science 241:1077; Van Brunt, (1990) Biotechnology 8:291; Wu and Wallace, (1989) Gene 4:560; Barringer, et al., (1990) Gene 89:117 and Sooknanan and Malek, (1995) Biotechnology 13:563. Additional methods, useful for cloning nucleic acids in the context of the invention, include Wallace, et al., U.S. Pat. No. 5,426,039. Improved methods of amplifying large nucleic acids by PCR are summarized in Cheng, et al., (1994) Nature 369:684 and the references therein.

Certain polynucleotides of the invention can be synthesized utilizing various solid-phase strategies involving mononucleotide- and/or trinucleotide-based phosphoramidite coupling chemistry. For example, nucleic acid sequences can be synthesized by the sequential addition of activated monomers and/or trimers to an elongating polynucleotide chain. See, e.g., Caruthers, et al., (1992) Meth Enzymol 211:3. In lieu of synthesizing the desired sequences, essentially any nucleic acid can be custom ordered from any of a variety of commercial sources, such as The Midland Certified Reagent Company (mcrc@oligos.com) (Midland, Tex.), The Great American Gene Company (available on the World Wide Web at genco.com) (Ramona, Calif.), ExpressGen, Inc. (available on the World Wide Web at expressgen.com) (Chicago, Ill.), Operon Technologies, Inc. (available on the World Wide Web at operon.com) (Alameda, Calif.) and many others.

TABLE 1
Sequence Identification.

| SEQ | Position | |
|---|---|---|
| ID | within SEQ | |
| NO: | ID NO: 3 | DESCRIPTION |
| 1 | 3272-3776 | TR3 ACS6 down-regulation sequence |
| 2 | 4332-4874 | TR4 ACS6 down-regulation sequence |
| 3 | 1-51,280 | Entire plasmid sequence |
| 4 | 3272-4874 | Comprising TR3, ADH1 intron 1, and TR4 |
| 5 | 1218-4874 | Comprising UBI1Zm promoter, UBI1Zm 5′UTR, |
| | | UBI1Zm Intron 1, TR3, ADH1 intron 1, and TR4 |
| 6 | 1218-7989 | Comprising UBI1Zm promoter, UBI1Zm 5′UTR, |
| | | UBI1Zm Intron 1, TR3, ADH1 intron 1, TR4, |
| | | ATTB2, FRT12, UBI1Zm promoter, UBI1Zm 5′ |
| | | UTR, UBI1Zm Intron 1, MO-PAT, and PinII |
| | | terminator |
| 7 | 1-8350 | Complete ACS down-regulation expression cassette |
| 8 | n/a | Sorghum bicolor PEP carboxylase promoter |
| 9 | n/a | genomic maize ACS3 sequence |
| 10 | n/a | UTR and CDS of maize ACS3 sequence |
| 11 | n/a | maize ACS3 amino acid sequence |
| 12 | n/a | rice ACS6 coding sequence |
| 13 | n/a | rice ACS6 amino acid sequence |

### EXAMPLES

The following examples are offered to illustrate, but not to limit, the claimed invention. Various modifications by persons skilled in the art are to be included within the spirit and purview of this application and scope of the appended claims.

### Example 1

### Protein Extraction

For total protein isolation, maize leaves are collected at the indicated times, quick-frozen in liquid nitrogen and ground to a fine powder. One ml of extraction buffer (20 mM HEPES (pH 7.6), 100 mM KCl, 10% Glycerol) is added to approximately 0.1 g frozen powder and mixed thoroughly. Samples are centrifuged 10 minutes at 10,000 rpm, the supernatant removed to a new tube and the concentration determined spectrophotometrically according to the methods of Bradford, (1976). See, Bradford, (1976) Anal. Biochem. 72:248-254.

### Chlorophyll Extraction

Leaves are frozen in liquid nitrogen and ground to a fine powder. Samples of approximately 0.1 g are removed to a 1.5 ml tube and weighed. Chlorophyll is extracted 5× with 1 ml (or 0.8 ml) of 80% acetone. Individual extractions are combined and the final volume adjusted to 10 ml (or 15 ml) with additional 80% acetone. Chlorophyll content (a+b) is determined spectrophotometrically according to the methods of Wellburn, (1994). See, Wellburn, (1994) J. Plant Physiol. 144:307-313.

### Measurement of Photosynthesis

Plants are grown in the field under normal and drought-stress conditions. Under normal conditions, plants are watered with an amount sufficient for optimum growth and yield. For drought-stressed plants, water may be limited for a period starting approximately one week before pollination and continuing through three weeks after pollination. During the period of limited water availability, drought-stressed plants may show visible signs of wilting and leaf rolling. The degree of stress may be calculated as % yield reduction relative to that obtained under well-watered conditions. Transpiration, stomatal conductance and CO₂ assimilation are determined with a portable TPS-1 Photosynthesis System (PP Systems, Amesbury, Mass.). Each leaf on a plant may be measured, e.g. at forty days after pollination. Values typically represent a mean of six determinations.

### DNA and RNA Purification

For total nucleic acid isolation, maize leaves are collected at desired times, quick-frozen in liquid nitrogen and ground to a fine powder. Ten ml of extraction buffer (100 mM Tris (pH 8.0), 50 mM EDTA, 200 mM NaCl, 1% SDS, 10 μl/ml β-mercaptoethanol) is added and mixed thoroughly until thawed. Ten ml of Phenol/Chloroform (1:1, vol:vol) is added and mixed thoroughly. Samples are centrifuged 10 min at 8,000 rpm, the supernatant is removed to a new tube and the nucleic acid is precipitated at −20° C. following addition of 1/10 vol 3M sodium acetate and 1 vol isopropanol. Total nucleic acid is pelleted by centrifugation at 8,000 rpm and resuspended in 1 ml TE. One half of the prep is used for DNA purification and the remaining half is used for RNA purification. Alternatively, DNA or total nucleic acids can be extracted from 1 cm² of seedling leaf, quick-frozen in liquid nitrogen and ground to a fine powder. 600 μl of extraction buffer [100 mM Tris (pH 8.0), 50 mM EDTA, 200 mM NaCl, 1% SDS, 10 μl/ml β-mercaptoethanol] is added and the sample mixed. The sample is extracted with 700 μl phenol/chloroform (1:1) and centrifuged for 10 minutes at 12,000 rpm. DNA is precipitated and resuspended in 600 μl H2O.

For DNA purification, 500 μg Dnase-free Rnase is added to the tube and incubated at 37° C. for 1 hr. Following Rnase digestion, an equal volume of Phenol/Chloroform (1:1, vol:vol) is added and mixed thoroughly. Samples are centrifuged 10 min at 10,000 rpm, the supernatant is removed to a new tube and the DNA precipitated at −20° C. following addition of 1/10 vol 3M sodium acetate and 1 vol isopropanol. DNA is resuspended in sterile water and the concentration is determined spectrophotometrically. To determine DNA integrity, 20 mg of DNA is separated on a 1.8% agarose gel and visualized following staining with ethidium bromide.

RNA is purified by 2 rounds of LiCl₂ precipitation according to methods described by Sambrook, et al., supra.

### Real-Time RT-PCR Analysis

Fifty μg total RNA is treated with RQ1™ DNase (Promega) to ensure that no contaminating DNA is present. Two μg total RNA is used directly for cDNA synthesis using the Omniscript™ reverse transcription kit (Qiagen) with oligo-dT(20) as the primer.

Analysis of transcript abundance is accomplished using the QuantiTect™ SYBR Green PCR kit (Qiagen). Reactions contain 1× buffer, 0.5 μl of the reverse transcription reaction (equivalent to 50 ng total RNA) and 0.25 μM (final concentration) forward and reverse primers in a total reaction volume of 25 μl.

Reactions are carried out using an ABI PRISM 7700 sequence detection system under the following conditions: 95° C./15 minutes (1 cycle); 95 C/30 sec, 62° C./30 sec, 72° C./2 minute (50 cycles); 72° C./5 minutes (1 cycle). Each gene is analyzed a minimum of four times.

Primer combinations are initially run and visualized on an agarose gel to confirm the presence of a single product of the correct size. Amplification products are subcloned into the pGEM®-T Easy Vector System (Promega) to use for generation of standard curves to facilitate conversion of expression data to a copy/μg RNA basis.

### Ethylene Determination

Ethylene may be measured from leaves, such as the second fully-expanded leaf of seedlings at the 4-leaf stage or the terminal 15 cm of leaves of plants 20, 30 or 40 days after pollination (DAP). Leaves are harvested at the indicated time or times and allowed to recover between moist paper towels for 2 hours prior to collecting ethylene. Leaves are placed into glass vials and capped with a rubber septum. Following a 3- to 4-hour incubation, 0.9 mL of headspace is sampled from each vial.

Ethylene may be measured from developing kernels at four time points: 14, 21, 27 and 29 days after pollination (DAP). Kernels are harvested and incubated in well-circulated air for 2 hr to liberate any stress ethylene. Kernels are then placed into 5 mL glass vials and immediately sealed with airtight subaseal stoppers or crimp tops. The vials are then incubated in the dark for 24 h at 28° C. Following this incubation, 0.2 mL of headspace is sampled from each vial.

Ethylene content is measured using an GC6890 series gas chromatography system with FID detection (Agilent Technologies, Palo Alto, Calif.) with the following parameters: Column J&W Porous Layer Open Tubular (PLOT) with HP-AL/M stationary phase; size 50 m×0.535 mm×15 μm; oven temperature 75° C. isothermal; run time 2 minutes; injector 250° C. splitless, pressure 22 psi. Standards used are from Praxair (Danbury, Conn.) for 10 ppm, 50 ppm and 100 ppm ethylene in an air balance. The limit of detection (LOD) is approximately 0.1 ppm; the limit of quantitation (LOQ) is approximately 0.5 ppm.

### Western Blot Analysis

Leaves are collected at the indicated times and ground in liquid nitrogen to a fine powder. One ml of extraction buffer [20 mM HEPES (pH 7.6), 100 mM KCl, 10% glycerol, 1 mM PMSF] is added to approximately 0.1 g frozen powder and mixed thoroughly. Cell debris is pelleted by centrifugation at 10,000 rpm for 10 min and the protein concentration determined as described (Bradford, 1976). Antiserum raised against the large subunit of rice Rubisco is obtained from Dr. Tadahiko Mae (Tohoku University, Sendai, Japan). Protein extracts are resolved using standard SDS-PAGE and the protein transferred to 0.22 μm nitrocellulose membrane by electroblotting. Following transfer, the membranes are blocked in 5% milk, 0.01% thimerosal in TPBS (0.1% TWEEN® 20, 13.7 mM NaCl, 0.27 mM KCl, 1 mM Na2HPO4, 0.14 mM KH2PO4) followed by incubation with primary antibodies diluted typically 1:1000 to 1:2000 in TPBS with 1% milk for 1.5 hrs. The blots are then washed twice with TPBS and incubated with goat anti-rabbit horseradish peroxidase-conjugated antibodies (Southern Biotechnology Associates, Inc.) diluted to 1:5000 to 1:10,000 for 1 hr. The blots are washed twice with TPBS and the signal detected typically between 1 to 15 min using chemiluminescence (Amersham Corp).

### Example 2

### ACC Synthase Down-Regulation by Hairpin RNA Expression

As noted previously, plant cells and plants can be modified by introduction of an ACC synthase polynucleotide sequence configured for RNA silencing or interference. This example describes hairpin RNA expression cassettes for modifying ethylene production, drought tolerance, NUE, seed or biomass yield, density tolerance or other phenotypes, e.g., in maize. As noted previously, down-regulation of ACC synthase(s), e.g., by hairpin RNA (hpRNA) expression, can result in plants or plant cells having reduced expression (up to and including no detectable expression) of one or more ACC synthases.

Expression of hpRNA molecules specific for one or more ACC synthase genes (e.g., ACC synthase promoters, other untranslated regions or coding regions) in plants can alter phenotypes such as ethylene production, drought tolerance, density tolerance, seed or biomass yield and/or nitrogen use efficiency of the plants, through RNA interference.

An hpRNA construct as described herein is generated by linking a ubiquitin promoter to a portion of the coding sequence of an ACS gene, such as the ACS6 gene and its inverted repeat sequence. Each construct is transformed into maize using Agrobacterium-mediated transformation techniques or another known transformation method. Nucleic acid molecules and methods for preparing the constructs and transforming maize are as previously described and known in the art; see, e.g., the sections herein entitled “Plant Transformation Methods,” “Other Nucleic Acid and Protein Assays” and the following example “Transformation of Maize”.

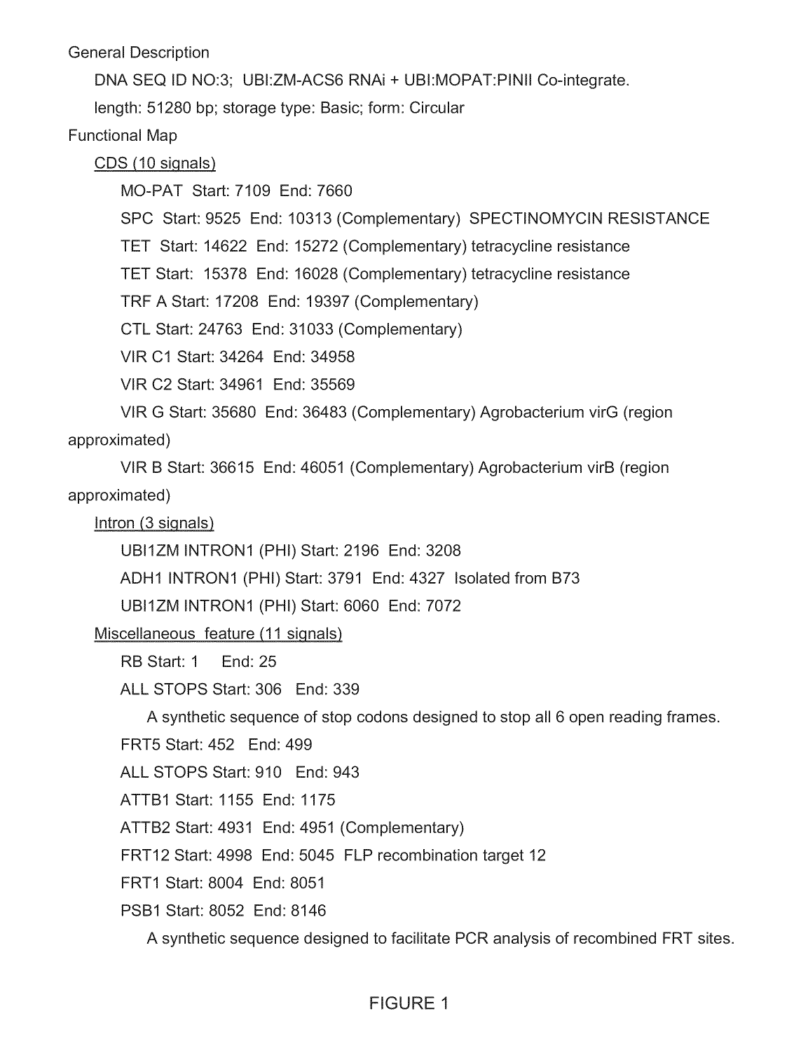

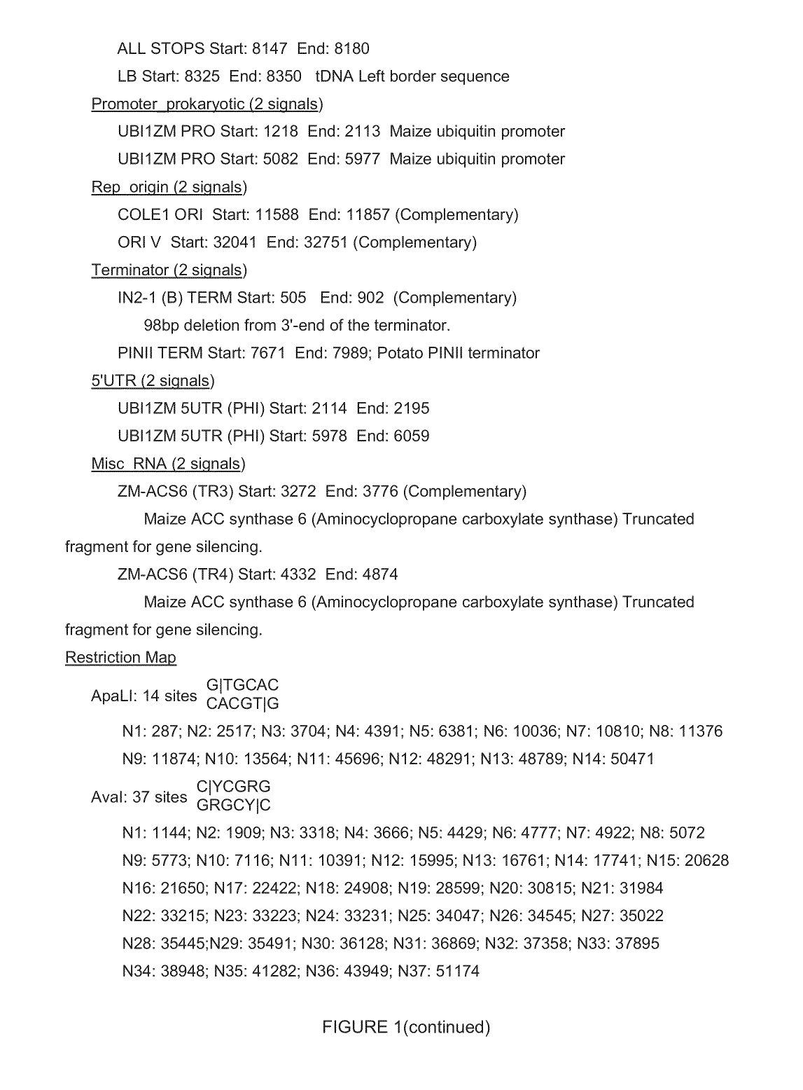

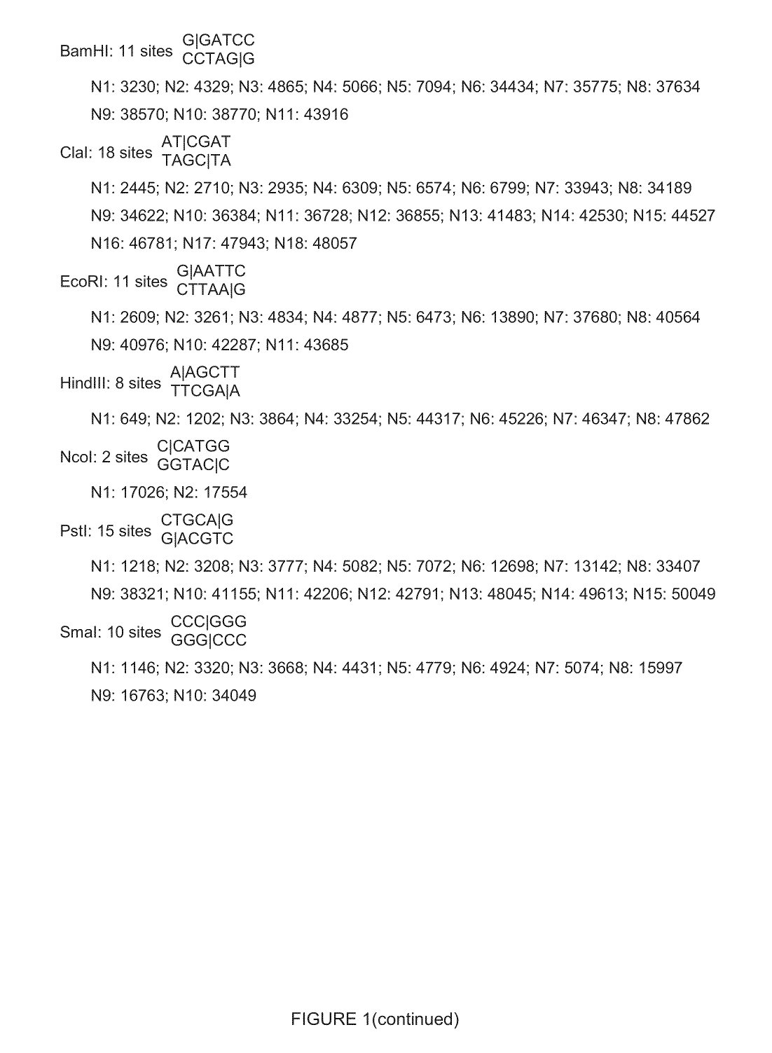

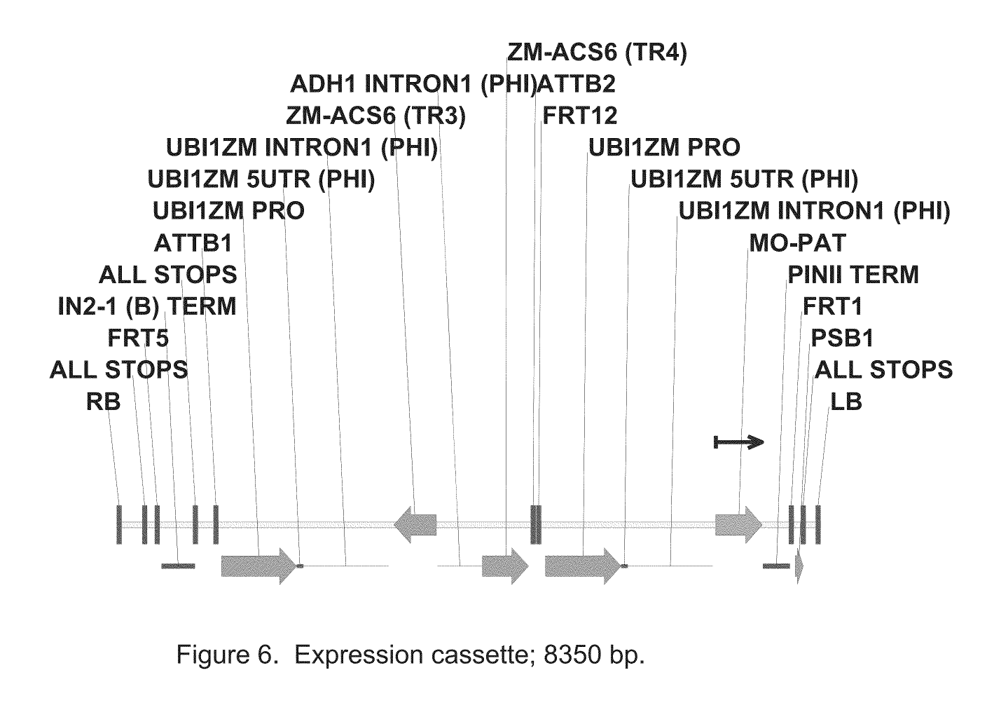

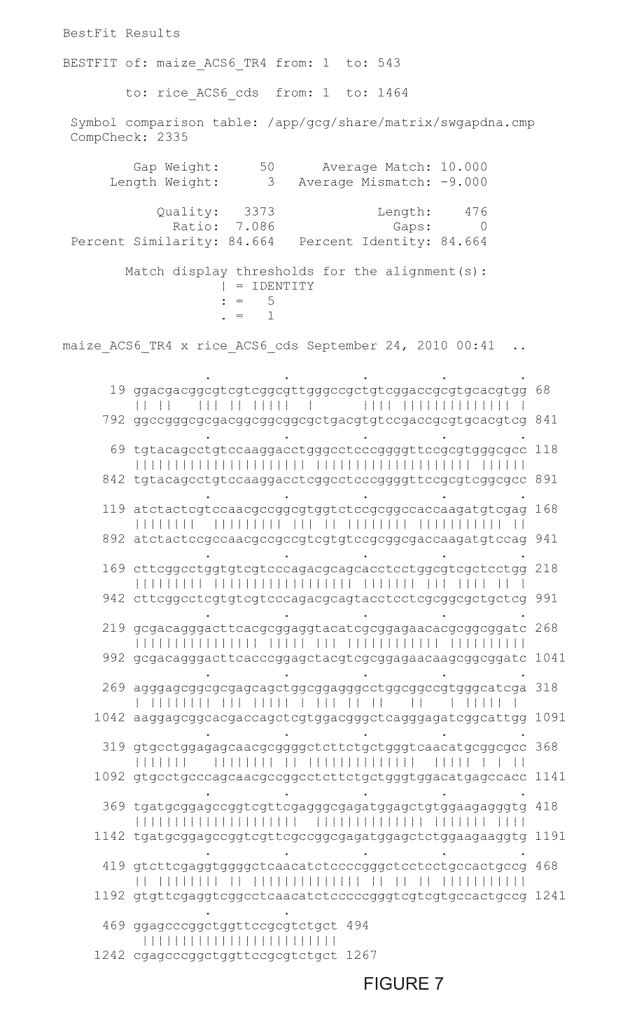

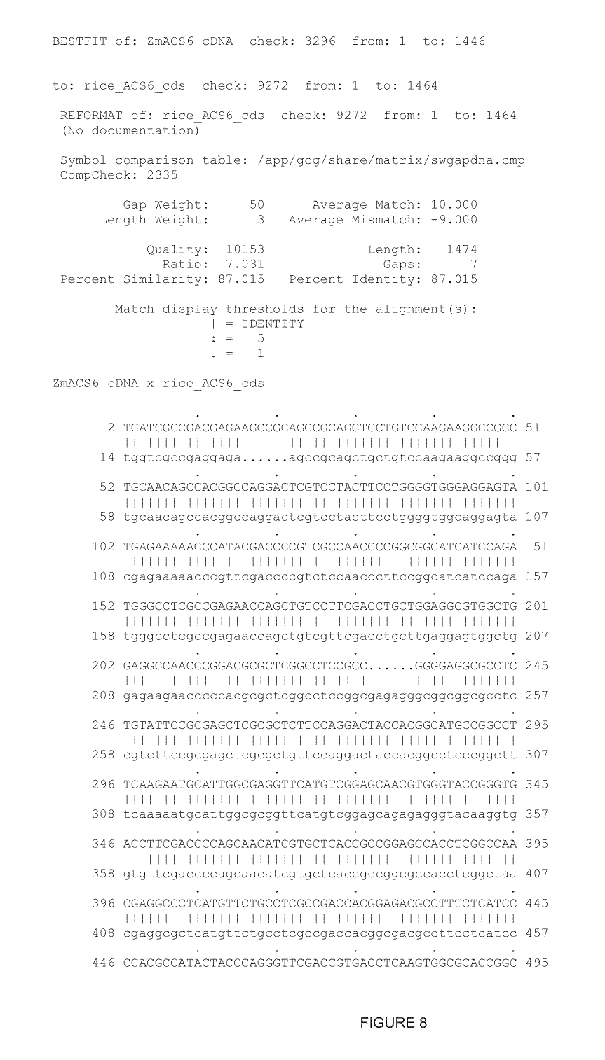

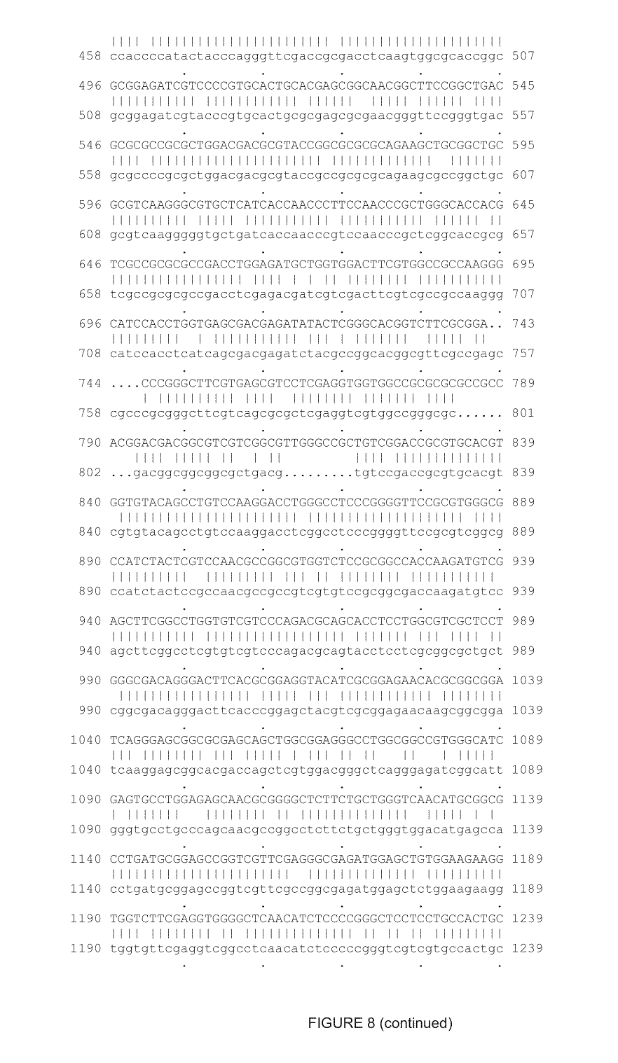

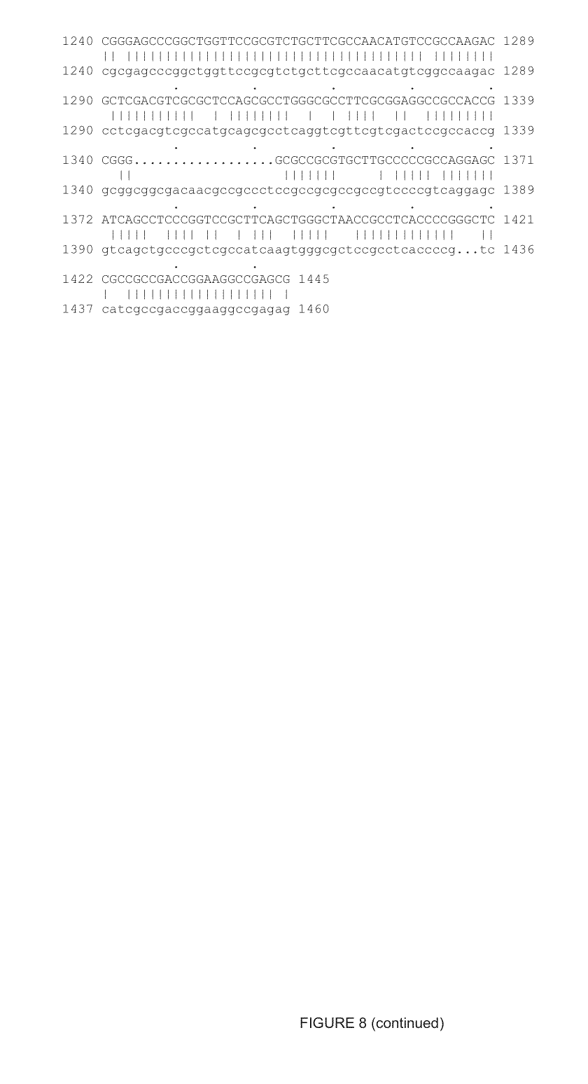

Expression of hpRNA targeting one or more ACC synthase genes, such as an ACS6 coding sequence, may result in maize plants that display no detrimental effects in vegetative and reproductive growth. Sequence of a plasmid comprising such an hpRNA construct a construct of the invention is provided in SEQ ID NO: 3. FIG. 6 is a schematic of a representative expression cassette; the expression cassette sequence is provided in SEQ ID NO: 7. FIG. 1 provides a listing of features of a plasmid comprising a representative expression cassette.

### Example 3

### Yield Evaluation—Season 1

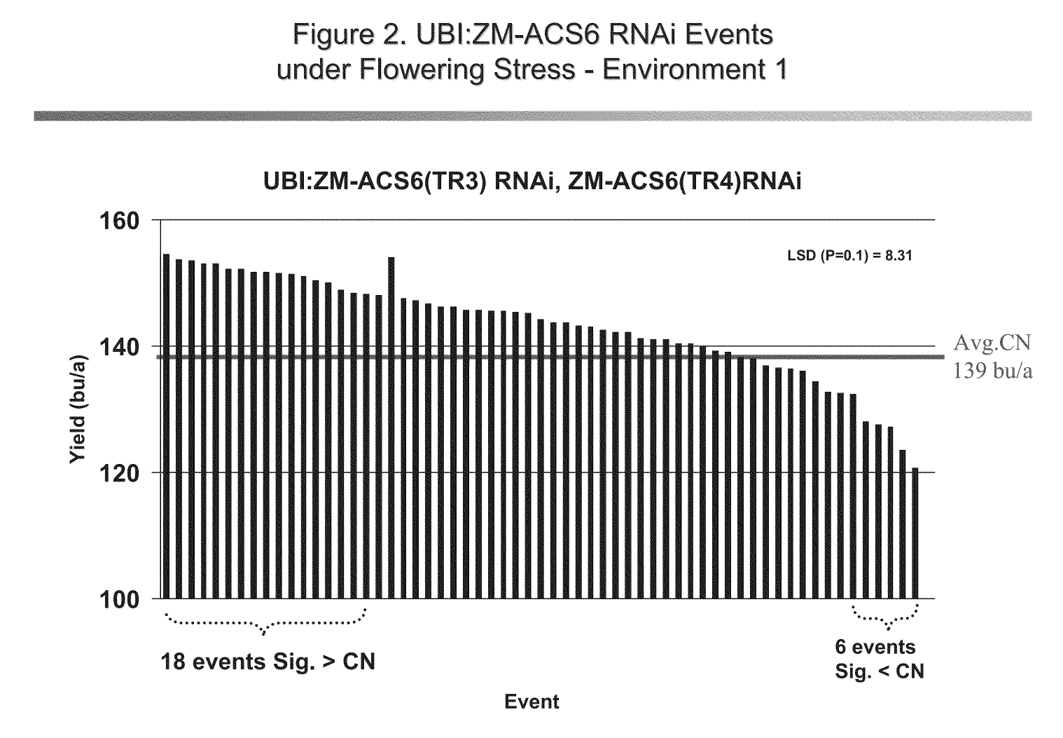

Transformed plants of genetic background #1, comprising the sequence of SEQ ID NO: 4 were evaluated for yield under four environments. Eight reps were grown under flowering stress in Environment 1; 6 reps were grown under grain fill stress in Environment 2; 6 reps were grown under grain fill stress in Environment 3 and 4 reps were grown under rain-fed conditions in Environment 4. Yields were compared with a highly repeated construct null (CN) comprising non-transgenic segregants of plants transformed with the construct. The data are shown in FIGS. 2-5.

FIG. 2 shows the yield of transformed plants of the invention under flowering stress in Environment 1. Each bar represents a separate transformation event. Average yield of transgene-negative segregants is shown (139 bu/a) as control (CN). A total of 74% of the events yielded nominally more than the control plants. Plants representing 18 transgenic events outyielded the control at P<0.10.

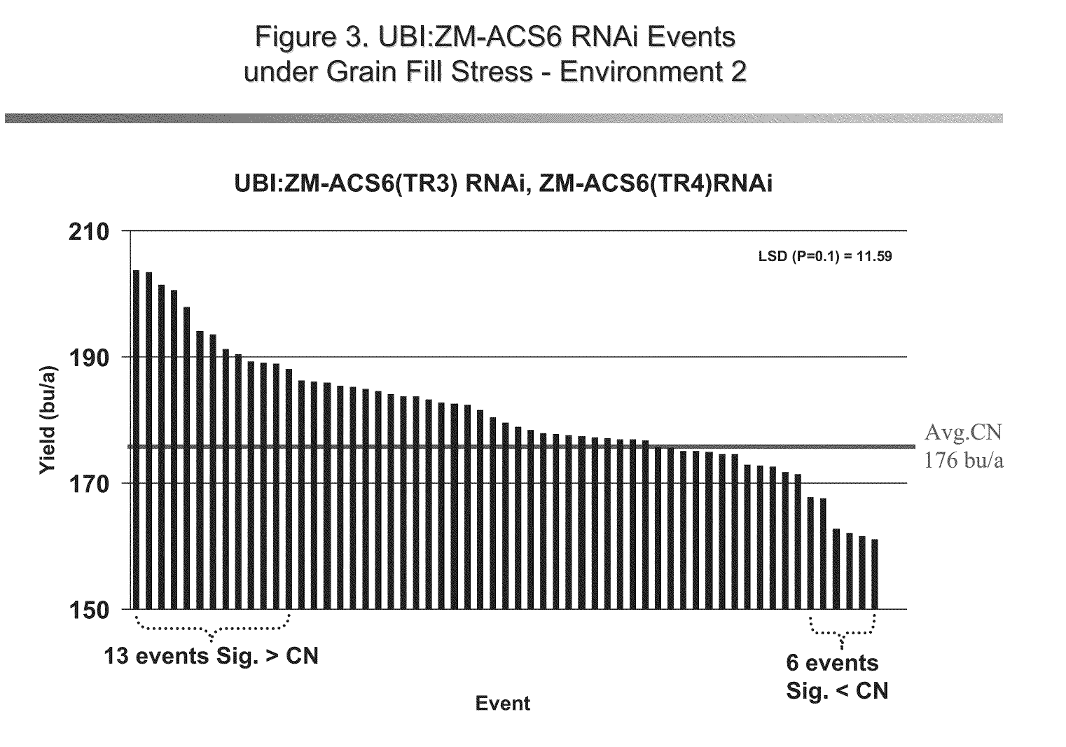

FIG. 3 shows the yield of transformed plants of the invention under grain-fill stress in Environment 2. Each bar represents a separate transformation event. Average yield of transgene-negative segregants is shown (176 bu/a) as control (CN). Thirteen events out-yielded the CN at P<0.10. Of these, eight had also shown significant improvement under flowering stress.

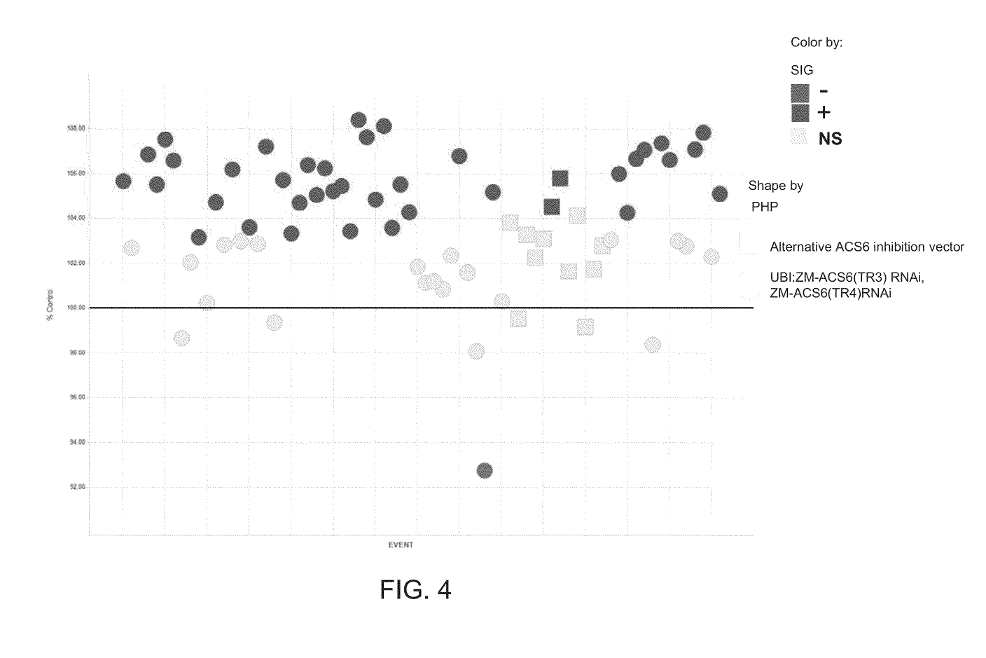

FIG. 4 shows the yield, as a percent of control, of transformed plants of the invention (indicated by a circle), as well as plants transformed using an alternative ACS6 down-regulation vector (indicated by a square) under grain fill stress in Environment 3. Each data point represents a separate transformation event. NS=not significant. The control plants are bulked transgene-negative segregants. As can be seen, 64% of the events of the invention had significantly superior yield; only 17% of the alternative ACS6 down-regulation events had significantly superior yield, relative to the control.

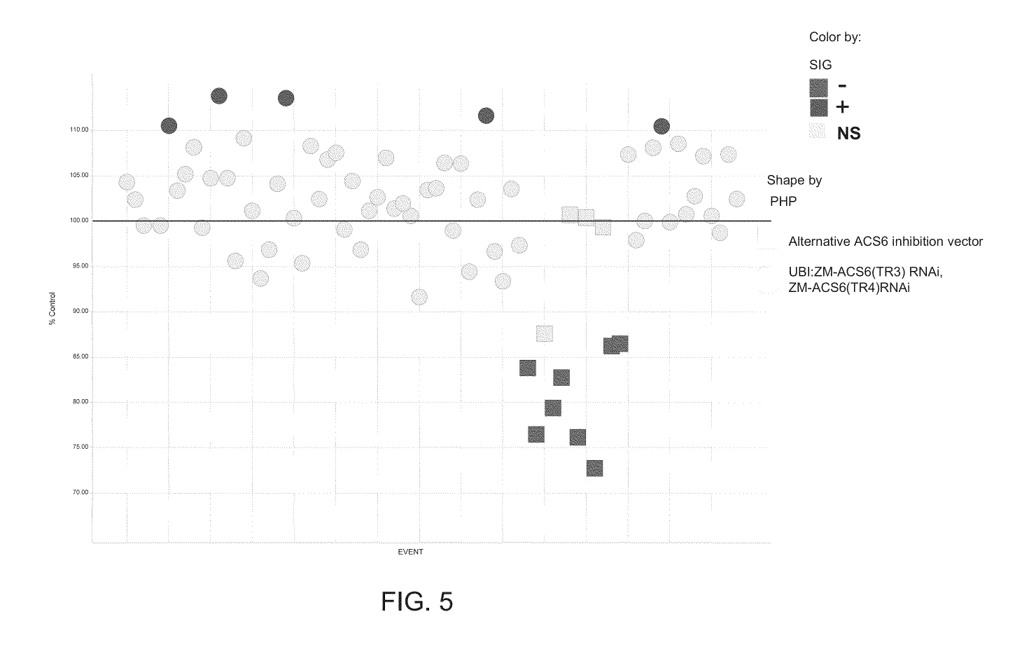

FIG. 5 shows the yield, as a percent of control, of transformed plants of the invention (indicated by a circle), as well as plants transformed using an alternative ACS6 down-regulation construct (indicated by a square) under rain-fed conditions in Environment 4. Each data point represents a separate transformation event. NS=not significant. The control plants are bulked transgene-negative segregants. As can be seen, all points exhibiting statistically significant increases in yield represent events as disclosed herein. In addition, all points exhibiting statistically significant decreases in yield are events containing the alternative ACS6 down-regulation construct.

Without being limited to any particular theory, the construct as herein disclosed provides improvement in yield and other phenotypic traits by modulating ACS expression. For example, inclusion of an intron (e.g., Adh1 intron) within the ACS6 hairpin may modulate ACS6 downregulation within an effective range. Alternatively or additionally, the construct of the invention may impact expression of other genes, for example ACS2 and/or ACS3.

### Example 4

### Screening of Gaspe Bay Flint Derived Maize Lines Under Nitrogen Limiting Conditions

Transgenic plants will contain two or three doses of Gaspe Flint-3 with one dose of GS3 (GS3/(Gaspe-3)2× or GS3/(Gaspe-3)3×) and will segregate 1:1 for a dominant transgene. Transgenic GS3×Gaspe T1 seeds and their respective nulls will be planted in 4-inch pots containing TURFACE®, a commercial potting medium and watered four times each day with 1 mM KNO₃ growth medium and with 2 mM (or higher) KNO₃ growth medium. After emergence, plants will be sampled to determine which are transgenic and which are nulls. At anthesis, plants are harvested and dried in a 70° C. oven for 72 hours and the shoot and ear dry weight determined. Results are analyzed for statistical significance. Expression of a transgene results in plants with improved nitrogen use efficiency in 1 mM KNO₃ when compared to a transgenic null. Increase in biomass, greenness and/or ear size at anthesis indicates increased NUE.

### Example 5

### NUE Assay

Seeds of Arabidopsis thaliana (control and transgenic line), ecotype Columbia, are surface sterilized (Sánchez, et al., 2002) and then plated on to Murashige and Skoog (MS) medium containing 0.8% (w/v) Bacto™-Agar (Difco). Plates are incubated for 3 days in darkness at 4° C. to break dormancy (stratification) and transferred thereafter to growth chambers (Conviron, Manitoba, Canada) at a temperature of 20° C. under a 16-h light/8-h dark cycle. The average light intensity is 120 μE/m2/s. Seedlings are grown for 12 days and then transferred to soil based pots. Potted plants are grown on a nutrient-free soil LB2 Metro-Mix® 200 (Scott's Sierra Horticultural Products, Marysville, Ohio, USA) in individual 1.5-in pots (Arabidopsis system; Lehle Seeds, Round Rock, Tex., USA) in growth chambers, as described above. Plants are watered with 0.6 or 6.5 mM potassium nitrate in the nutrient solution based on Murashige and Skoog (MS free Nitrogen) medium. The relative humidity is maintained around 70%. Sixteen to eighteen days later, plant shoots are collected for evaluation of biomass and SPAD (chlorophyll) readings.

### Example 6

### Sucrose Growth Assay

The Columbia line of Arabidopsis thaliana is obtained from the Arabidopsis Biological Resource Center (Columbus, Ohio). For early analysis (Columbia and T3 transgenic lines), seed are surface-sterilized with 70% ethanol for 5 minutes followed by 40% Clorox® for 5 minutes and rinsed with sterile deionized water. Surface-sterilized seed are sown onto square Petri plates (25 cm) containing 95 mL of sterile medium consisting of 0.5 Murashige and Skoog (1962) salts (Life Technologies) and 4% (w/v) phytagel (Sigma). The medium contains no supplemental sucrose. Sucrose is added to medium in 0.1%, 0.5% and 1.5% concentration. Plates are arranged vertically in plastic racks and placed in a cold room for 3 days at 4° C. to synchronize germination. Racks with cold stratified seed are then transferred into growth chambers (Conviron, Manitoba, Canada) with day and night temperatures of 22 and 20° C., respectively. The average light intensity at the level of the rosette is maintained at 110 mol/m2/sec1 during a 16-hr light cycle development beginning at removal from the cold room (day 3 after sowing) until the seedlings are harvested on day 14. Images are taken and total fresh weight of root and shoot are measured.

### Example 7

### Low Nitrogen Seedling Assay Protocol

Seed of transgenic events are separated into transgene and null seed. Two different random assignments of treatments are made to each block of 54 pots arranged 6 rows of 9 columns using 9 replicates of all treatments. In one case null seed of 5 events of the same construct are mixed and used as control for comparison of the 5 positive events in this block, making up 6 treatment combinations in each block. In the second case, 3 transgenic positive treatments and their corresponding nulls are randomly assigned to the 54 pots of the block, making 6 treatment combinations for each block, containing 9 replicates of all treatment combinations. In the first case transgenic parameters are compared to a bulked construct null and in the second case transgenic parameters are compared to the corresponding event null. In cases where there are 10, 15 or 20 events in a construct, the events are assigned in groups of 5 events, the variances calculated for each block of 54 pots but the block null means pooled across blocks before mean comparisons are made.

Two seed of each treatment are planted in 4 inch, square pots containing TURFACE®-MVP on 8 inch, staggered centers and watered four times each day with a solution containing the following nutrients:

| | | | |
|---|---|---|---|
| 1 mM CaCl₂ | 2 mM MgSO₄ | 0.5 mM KH₂PO₄ | 83 ppm Sprint330 |
| 3 mM KCl | 1 mM KNO₃ | 1 uM ZnSO₄ | 1 uM MnCl₂ |
| 3 uM H₃BO₄ | 1 uM MnCl₂ | 0.1 uM CuSO₄ | 0.1 uM NaMoO₄ |

After emergence the plants are thinned to one seed per pot. Seedlings are harvested 18 days after planting. At harvest, plants are removed from the pots and the Turface washed from the roots. The roots are separated from the shoot, placed in a paper bag and dried at 70° C. for 70 hr. The dried plant parts (roots and shoots) are weighed and placed in a 50 ml conical tube with approximately 20 5/32 inch steel balls and ground by shaking in a paint shaker. Approximately, 30 mg of the ground tissue is hydrolyzed in 2 ml of 20% H₂O₂ and 6M H₂SO₄ for 30 minutes at 170° C. After cooling, water is added to 20 ml, mixed thoroughly, and a 50 μl aliquot removed and added to 950 μl 1M Na₂CO₃. The ammonia in this solution is used to estimate total reduced plant nitrogen by placing 100 μl of this solution in individual wells of a 96 well plate followed by adding 50 μl of OPA solution. Fluorescence, excitation=360 nM/emission=530 nM, is determined and compared to NH₄Cl standards dissolved in a similar solution and treated with OPA solution.

OPA solution−5 ul Mercaptoethanol+1 ml OPA stock solution
OPA stock−50 mg o-phthadialdehyde (OPA—Sigma #P0657) dissolved in 1.5 ml methanol+4.4 ml 1M Borate buffer pH9.5 (3.09 g H₃BO₄+1 g NaOH in 50 ml water)+0.55 ml 20% SDS

The following parameters are measured and means compared to null mean parameters using a Student's t test: total plant biomass; root biomass; shoot biomass; root/shoot ratio; plant N concentration; total plant N.

Variance is calculated within each block using a nearest neighbor calculation as well as by Analysis of Variance (ANOVA) using a completely random design (CRD) model. An overall treatment effect for each block is calculated using an F statistic by dividing overall block treatment mean square by the overall block error mean square.

### Example 8

### Transformation of Maize

### Biolistics

Polynucleotides contained within a vector can be transformed into embryogenic maize callus by particle bombardment, generally as described by Tomes, et al., Plant Cell, Tissue and Organ Culture: Fundamental Methods, Eds. Gamborg and Phillips, Chapter 8, pgs. 197-213 (1995) and as briefly outlined below. Transgenic maize plants can be produced by bombardment of embryogenically responsive immature embryos with tungsten particles associated with DNA plasmids. The plasmids typically comprise a selectable marker and a structural gene, or a selectable marker and an ACC synthase downregulation polynucleotide sequence or subsequence, or the like.

### Preparation of Particles

Fifteen mg of tungsten particles (General Electric), 0.5 to 1.8μ, preferably 1 to 1.8μ, and most preferably 1μ, are added to 2 ml of concentrated nitric acid. This suspension is sonicated at 0° C. for 20 minutes (Branson Sonifier Model 450, 40% output, constant duty cycle). Tungsten particles are pelleted by centrifugation at 10000 rpm (Biofuge) for one minute and the supernatant is removed. Two milliliters of sterile distilled water are added to the pellet, and brief sonication is used to resuspend the particles. The suspension is pelleted, one milliliter of absolute ethanol is added to the pellet and brief sonication is used to resuspend the particles. Rinsing, pelleting and resuspending of the particles are performed two more times with sterile distilled water and finally the particles are resuspended in two milliliters of sterile distilled water. The particles are subdivided into 250-μl aliquots and stored frozen.

### Preparation of Particle-Plasmid DNA Association

The stock of tungsten particles are sonicated briefly in a water bath sonicator (Branson Sonifier Model 450, 20% output, constant duty cycle) and 50 μl is transferred to a microfuge tube. The vectors are typically cis: that is, the selectable marker and the gene (or other polynucleotide sequence) of interest are on the same plasmid.

Plasmid DNA is added to the particles for a final DNA amount of 0.1 to 10 μg in 10 μL total volume and briefly sonicated. Preferably, 10 μg (1 μg/μL in TE buffer) total DNA is used to mix DNA and particles for bombardment. Fifty microliters (50 μL) of sterile aqueous 2.5 M CaCl₂ are added and the mixture is briefly sonicated and vortexed. Twenty microliters (20 μL) of sterile aqueous 0.1 M spermidine are added and the mixture is briefly sonicated and vortexed. The mixture is incubated at room temperature for 20 minutes with intermittent brief sonication. The particle suspension is centrifuged and the supernatant is removed. Two hundred fifty microliters (250 μL) of absolute ethanol are added to the pellet, followed by brief sonication. The suspension is pelleted, the supernatant is removed and 60 μl of absolute ethanol are added. The suspension is sonicated briefly before loading the particle-DNA agglomeration onto macrocarriers.

### Preparation of Tissue

Immature embryos of maize variety High Type II are the target for particle bombardment-mediated transformation. This genotype is the F1 of two purebred genetic lines, parents A and B, derived from the cross of two known maize inbreds, A188 and B73. Both parents were selected for high competence of somatic embryogenesis, according to Armstrong, et al., (1991) Maize Genetics Coop. News 65:92.

Ears from F1 plants are selfed or sibbed and embryos are aseptically dissected from developing caryopses when the scutellum first becomes opaque. This stage occurs about 9 to 13 days post-pollination and most generally about 10 days post-pollination, depending on growth conditions. The embryos are about 0.75 to 1.5 millimeters long. Ears are surface sterilized with 20% to 50% Clorox® for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite® and 8.5 mg/l AgNO₃. Chu, et al., (1975) Sci. Sin. 18:659; Eriksson, (1965) Physiol. Plant 18:976. The medium is sterilized by autoclaving at 121° C. for 15 minutes and dispensed into 100×25 mm Petri dishes. AgNO₃ is filter-sterilized and added to the medium after autoclaving. The tissues are cultured in complete darkness at 28° C. After about 3 to 7 days, most usually about 4 days, the scutellum of the embryo swells to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3 to 16 hours, preferably 4 hours, in complete darkness at 28° C. just prior to bombardment with particles associated with plasmid DNA.

To effect particle bombardment of embryos, the particle-DNA agglomerates are accelerated using a DuPont PDS-1000 particle acceleration device. The particle-DNA agglomeration is briefly sonicated and 10 μl are deposited on macrocarriers and the ethanol is allowed to evaporate. The macrocarrier is accelerated onto a stainless-steel stopping screen by the rupture of a polymer diaphragm (rupture disk). Rupture is effected by pressurized helium. The velocity of particle-DNA acceleration is determined based on the rupture disk breaking pressure. Rupture disk pressures of 200 to 1800 psi are used, with 650 to 1100 psi being preferred and about 900 psi being most highly preferred. Multiple disks are used to effect a range of rupture pressures.

The shelf containing the plate with embryos is placed 5.1 cm below the bottom of the macrocarrier platform (shelf #3). To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 in Hg. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/l thiamine HCl, 30 gm/l sucrose, 1 mg/l 2,4-dichlorophenoxyacetic acid, 2 gm/l Gelrite®, 0.85 mg/l Ag NO₃ and 3 mg/l bialaphos (Herbiace, Meiji). Bialaphos is added filter-sterilized. The embryos are subcultured to fresh selection medium at 10 to 14 day intervals. After about 7 weeks, embryogenic tissue, putatively transformed for both selectable and unselected marker genes, proliferates from a fraction of the bombarded embryos. Putative transgenic tissue is rescued and that tissue derived from individual embryos is considered to be an event and is propagated independently on selection medium. Two cycles of clonal propagation are achieved by visual selection for the smallest contiguous fragments of organized embryogenic tissue.

A sample of tissue from each event is processed to recover DNA. The DNA is restricted with a restriction endonuclease and probed with primer sequences designed to amplify DNA sequences overlapping the ACC synthase and non-ACC synthase portion of the plasmid. Embryogenic tissue with amplifiable sequence is advanced to plant regeneration.

For regeneration of transgenic plants, embryogenic tissue is subcultured to a medium comprising MS salts and vitamins (Murashige and Skoog, (1962) Physiol. Plant 15:473), 100 mg/l myo-inositol, 60 gm/l sucrose, 3 gm/l Gelrite®, 0.5 mg/l zeatin, 1 mg/l indole-3-acetic acid, 26.4 ng/I cis-trans-abscissic acid and 3 mg/l bialaphos in 100×25 mm Petri dishes and is incubated in darkness at 28° C. until the development of well-formed, matured somatic embryos is seen. This requires about 14 days. Well-formed somatic embryos are opaque and cream-colored and are comprised of an identifiable scutellum and coleoptile. The embryos are individually subcultured to a germination medium comprising MS salts and vitamins, 100 mg/l myo-inositol, 40 gm/l sucrose and 1.5 gm/l Gelrite® in 100×25 mm Petri dishes and incubated under a 16 hour light:8 hour dark photoperiod and 40 meinsteinsm⁻² sec⁻¹ from cool-white fluorescent tubes. After about 7 days, the somatic embryos germinate and produce a well-defined shoot and root. The individual plants are subcultured to germination medium in 125×25 mm glass tubes to allow further plant development. The plants are maintained under a 16 hour light: 8 hour dark photoperiod and 40 meinsteinsm⁻² sec-1 from cool-white fluorescent tubes. After about 7 days, the plants are well-established and are transplanted to horticultural soil, hardened off and potted into commercial greenhouse soil mixture and grown to sexual maturity in a greenhouse. An elite inbred line is used as a male to pollinate regenerated transgenic plants.

### Agrobacterium-Mediated

For Agrobacterium-mediated transformation, the method of Zhao, et al., may be employed as in PCT Patent Publication Number WO 1998/32326, the contents of which are hereby incorporated by reference. Briefly, immature embryos are isolated from maize and the embryos contacted with a suspension of Agrobacterium (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos re cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants.

### Example 9

### Expression of Transgenes in Monocots

A plasmid vector is constructed comprising a preferred promoter operably linked to an isolated polynucleotide comprising an ACC synthase polynucleotide sequence or subsequence. This construct can then be introduced into maize cells by the following procedure.

Immature maize embryos are dissected from developing caryopses derived from crosses of maize lines. The embryos are isolated 10 to 11 days after pollination when they are 1.0 to 1.5 mm long. The embryos are then placed with the axis-side facing down and in contact with agarose-solidified N6 medium (Chu, et al., (1975) Sci. Sin. Peking 18:659-668). The embryos are kept in the dark at 27° C. Friable embryogenic callus, consisting of undifferentiated masses of cells with somatic proembryoids and embryoids borne on suspensor structures, proliferates from the scutellum of these immature embryos. The embryogenic callus isolated from the primary explant can be cultured on N6 medium and sub-cultured on this medium every 2 to 3 weeks.

The plasmid p35S/Ac (Hoechst Ag, Frankfurt, Germany) or equivalent may be used in transformation experiments in order to provide for a selectable marker. This plasmid contains the Pat gene (see, EP Patent Publication Number 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell, et al., (1985) Nature 313:810-812) and comprises the 3′ region of the nopaline synthase gene from the T-DNA of the Ti plasmid of Agrobacterium tumefaciens.

The particle bombardment method (Klein, et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic™ PDS-1000/He biolistic particle delivery system (Bio-Rad Instruments, Hercules, Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains glufosinate (2 mg per liter) and lacks casein or proline. The tissue continues to grow slowly on this medium. After an additional 2 weeks the tissue can be transferred to fresh N6 medium containing glufosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839).

### Example 10

### Expression of Transgenes in Dicots

Soybean embryos are bombarded with a plasmid comprising a preferred promoter operably linked to a heterologous nucleotide sequence comprising an ACC synthase polynucleotide sequence or subsequence (e.g., SEQ ID NOS: 1 and 2), as follows. To induce somatic embryos, cotyledons of 3 to 5 mm in length are dissected from surface-sterilized, immature seeds of the soybean cultivar A2872, then cultured in the light or dark at 26° C. on an appropriate agar medium for six to ten weeks. Somatic embryos producing secondary embryos are then excised and placed into a suitable liquid medium. After repeated selection for clusters of somatic embryos that multiply as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are sub-cultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A DuPont Biolistic™ PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette of interest, comprising the preferred promoter and a heterologous ACC synthase polynucleotide, can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M) and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×5 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 11

### Field Trials Under Nitrogen Stress and Normal Nitrogen Conditions

Corn hybrids containing an ACS down-regulation construct transgene are planted in the field under nitrogen-stress and normal-nitrogen conditions. Under normal nitrogen, a total of 250 lbs nitrogen is applied in the form of urea ammonium nitrate (UAN). Nitrogen stress is achieved through depletion of soil nitrogen reserves by planting corn with no added nitrogen for two years. Soil nitrate reserves are monitored to assess the level of depletion. To achieve the target level of stress, UAN is applied by fertigation or sidedress between V2 and VT growth stages, for a total of 50-150 lbs nitrogen.

Events from the construct are nested together with the null to minimize the spatial effects of field variation. Multiple reps are planted. The seed yield of events containing the transgene is compared to the yield of a transgenic null. Statistical analysis is conducted to assess whether there is a significant improvement in yield compared with the transgenic null, taking into account row and column spatial effects.

Differences in yield, yield components or other agronomic traits between transgenic and non-transgenic plants in reduced-nitrogen fertility plots may indicate improvement in nitrogen utilization efficiency contributed by expression of a transgenic event. Similar comparisons are made in plots supplemented with recommended nitrogen fertility rates. Effective transgenic events may achieve similar yields in the nitrogen-limited and normal nitrogen environments or may perform better than the non-transgenic counterpart in low-nitrogen environments.

### Example 12

### ACS6 Down-Regulation Construct Improves Yield in Reduced-Nitrogen Conditions

Plants comprising a downregulation construct comprising SEQ ID NO: 4 were planted in the field under nitrogen-stress and normal-nitrogen conditions. Nitrogen stress was achieved through targeted depletion of soil nitrogen reserves by previous corn production and/or limited application of nitrogen fertilizer. In addition to cropping history, soil type and other environmental factors were taken into consideration in creating appropriate nitrogen-stress conditions.

The grain yield of plants containing the transgene was compared to the yield of a wild-type or transgenic null. The test used a randomized complete block design with six replications. Statistical analysis was conducted using ASRemI to assess differences in yield, taking into account row and column spatial effects and autoregressive (AR1) adjustments.

Table 2 provides yield data in bushels/acre for plants representing 19 transformation events under nitrogen-stress conditions in two geographic locations. Yields marked with an asterisk are significantly greater than the control at P<0.1.

TABLE 2

| Event | Location 1 | Location 2 |
|---|---|---|
| 2.12 | 121 | 202* |
| 2.29 | 124* | 199 |
| 2.32 | 123 | 211* |
| 113.2.7 | 124* | 206* |
| 4.3 | 124* | 204* |
| 4.8 | 125* | 203* |
| 1.23 | 127* | 208* |
| 1.44 | 126* | 207* |
| 2.15 | 124* | 205* |
| 2.2 | 124 | 201 |
| 2.24 | 124* | 198 |
| 2.38 | 125* | 204* |
| 2.49 | 123 | 202* |
| 1.14 | 125* | 210* |
| 2.18 | 126* | 206* |
| 2.22 | 124* | 208* |
| 2.8 | 125* | 205* |
| 2.1 | 125* | 206* |
| 66.2.7 | 124* | 202* |
| Control | 120 | 197 |

Additional measurements were taken at Location 2, as follows. Average yield of the transgenic plants under normal-nitrogen conditions was 232 bushels per acre; under nitrogen-stress conditions, the average yield was 203 bushels per acre. Under nitrogen stress, growing-degree-units to pollen shed was 1273 compared to 1330 under normal-nitrogen conditions. In addition, plants grown in the nitrogen-stress environment showed a reduction in anthesis-silking interval (ASI) of 18. Barren count in the low-nitrogen environment was 1 on a 1 to 10 scale, where 10 is least favorable.

### Example 13

### Yield Evaluation—Season 2

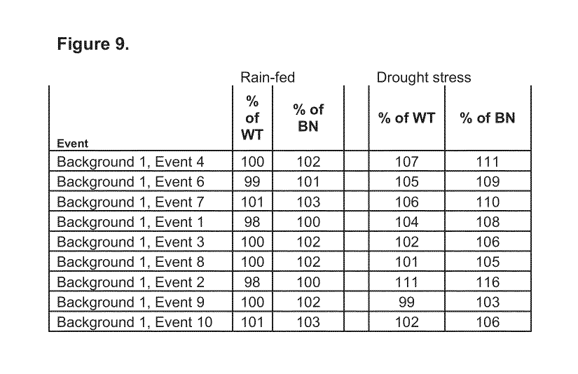

Maize hybrids were generated by crossing tester lines with plants comprising an ACS downregulation construct substantially as described in FIG. 6. These plants represented nine separate transformation events in Background 1. Hybrids were evaluated for yield in multiple locations in Season 2. Grain yield was compared to that of untransformed hybrids (WT, wild-type) and bulked nontransgenic segregants (BN, bulk null) from all constructs in the block sharing the same background. Results are shown in FIG. 9.

### Example 14

### Yield Evaluation—Season 3

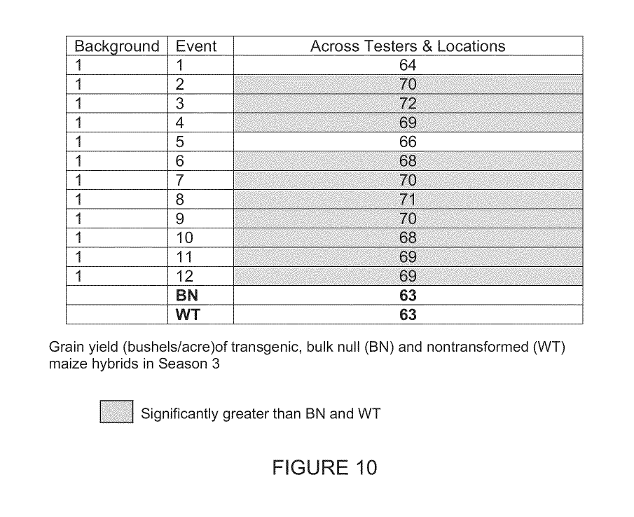

Four maize hybrids were generated by crossing tester lines with plants comprising an ACS downregulation construct substantially as described in FIG. 6. Twelve separate transformation events were individually represented in each hybrid combination. The hybrids were grown in a split-plot experimental design with four replications in each of four testing sites. Bulked nontransgenic segregants (BN, bulk null) from all constructs in a block, and/or untransformed hybrids (WT, wild-type), served as controls at each site. Grain yield (bushels/acre) and plant height data were collected and analyzed. Significance was determined at P<0.1 using BLUP (Best Linear Unbiased Predictor) analysis (Henderson, (1975) Biometrics 31(2):423-447; Robinson, (1991) Statistical Science 6(1):15-32). As shown in FIG. 10, ten of twelve transgenic events yielded more than both the bulk null and wild-type controls.

### Example 15

ACC synthase (ACS) catalyzes the synthesis of 1-aminocyclopropane-1-carboxylic acid (ACC) from S-adenosyl-L-methionine (SAM), the first committed step of ethylene biosynthesis. This step is rate-limiting for ethylene formation; expression of ACS is tightly regulated at both the transcriptional and post-transcriptional levels (review, Kende, (1993) Ann. Rev. Plant Physiol. Plant Mol. Biol. 44:283-307).

In Arabidopsis, 12 genes were named as AtACS. Later AtACS3 was identified as a pseudogene and AtACS10 and AtACS12 were found to encode not ACS but aminotransferases (Yamagami, et al., (2003) Journal of Biol. Chem. 278(49):49102-49112. In maize, three ACC synthases (ZmACS6, ZmACS2 and ZmACS7) have been previously studied (Gallie and Young, (2004) Mol. Gen. Gen. 271:267-281; Wang, et al., (2002) Plant Cell 14:S131-151). A previously unreported maize ACC synthase, designated ZmACS3, is shown in SEQ ID NOS: 9-11. Modulation of expression of ZmACS3, particularly downregulation of ZmACS3, alone or in combination with modulation of other genes, may reduce ethylene production, resulting in increased growth rate and improved stress tolerance in plants. For example, suppression of expression of both ZmACS6 and ZmACS3 in maize may result in higher growth rate and improved yield under optimal and/or stress (e.g. drought) conditions.

Methods and compositions to modulate plant development may use DNA, RNA or protein of or derived from the ZmACS3 gene. Certain embodiments provide an isolated polypeptide comprising an amino acid sequence selected from the group consisting of: (a) the polypeptide comprising the amino acid sequence of SEQ ID NO:11; (b) a polypeptide having at least 80% sequence identity to the full length of SEQ ID NO: 11, wherein the polypeptide has ACC synthase activity; (c) a polypeptide encoded by a polynucleotide that hybridizes under stringent conditions to a polynucleotide comprising the complement of SEQ ID NO: 10, wherein the stringent conditions comprise 50% formamide, 1 M NaCl, 1% SDS at 37° C. and a wash in 0.1×SSC at 60° C. to 65° C. and (d) the polypeptide having at least 70 consecutive amino acids of SEQ ID NO:11, wherein the polypeptide retains ACC synthase activity.

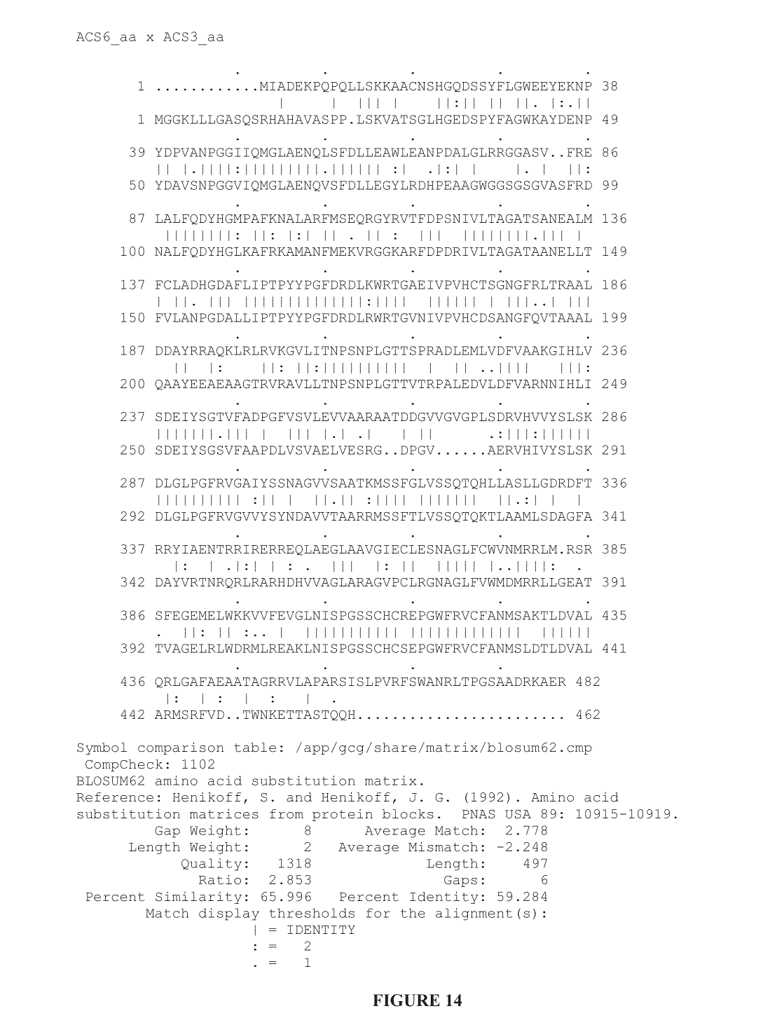

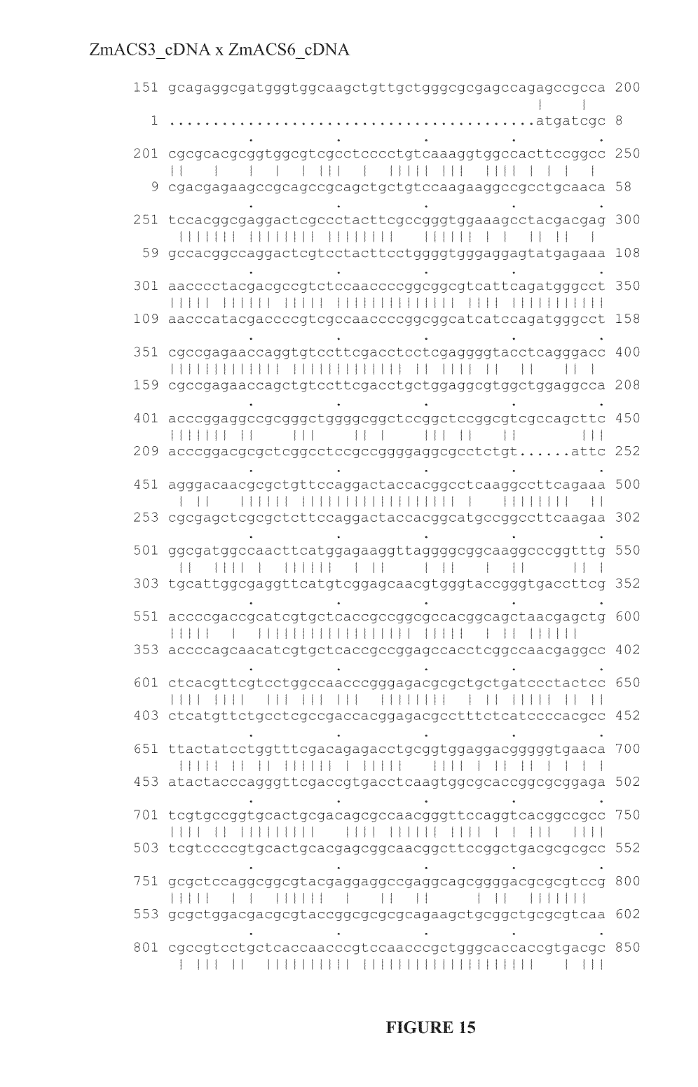

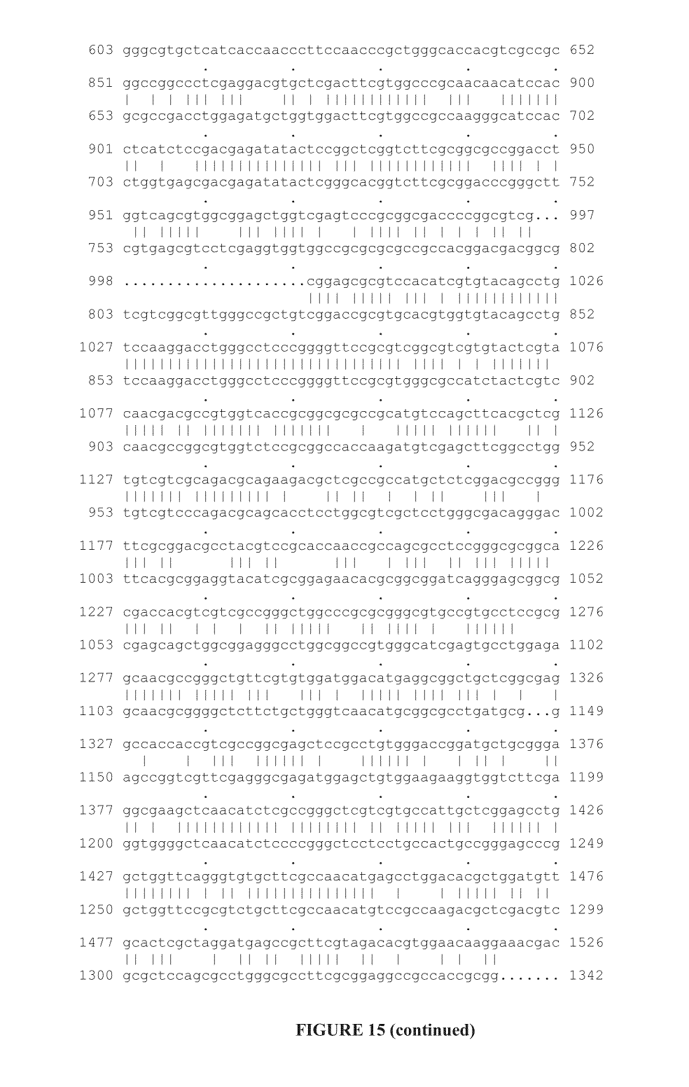

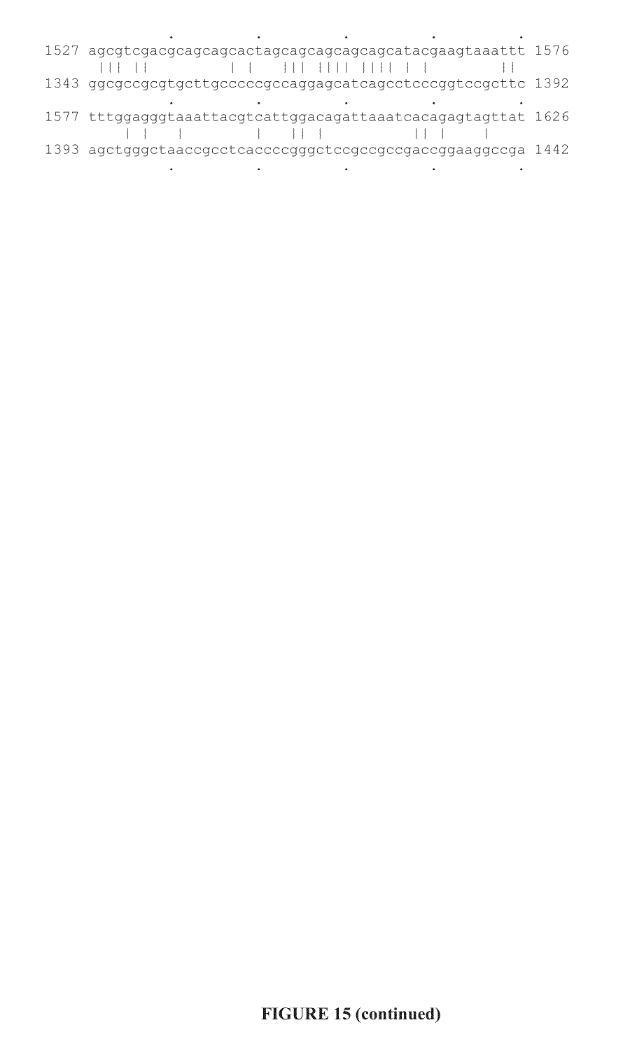

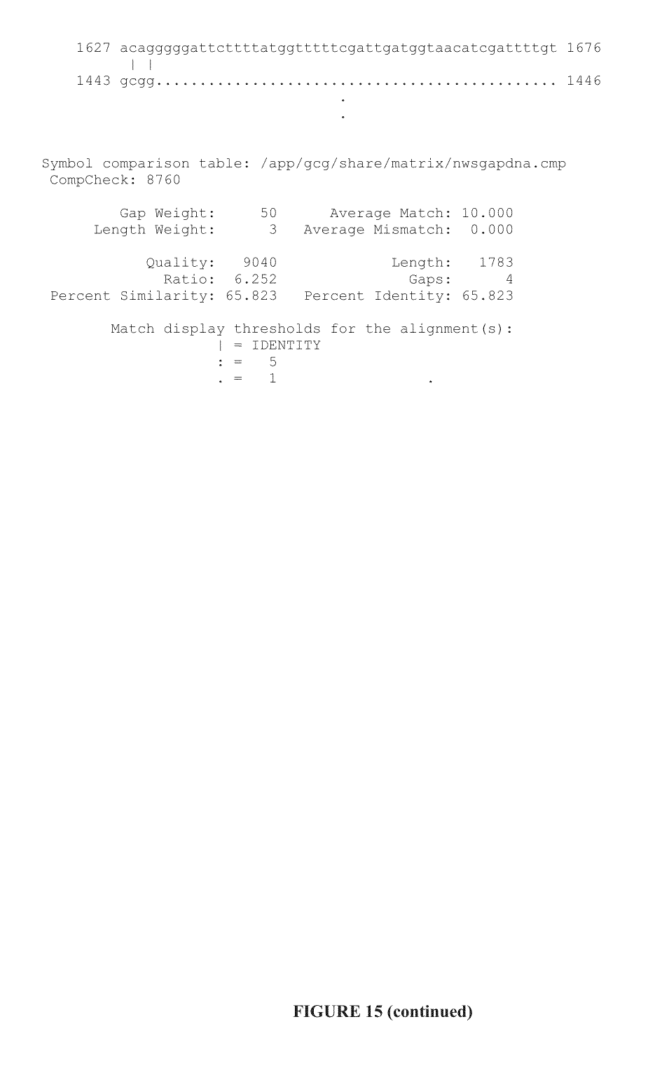

The ACS3 polypeptide shares moderate (59%) identity with the ACS6 protein; see, FIG. 14 for an alignment. Identity between the cDNA sequences of ACS6 and ACS3 is approximately 66%; see, FIG. 15 for an alignment.

By “ACS3 activity” or “ACC Synthase 3 activity” is intended the ACS3 polypeptide has exemplary activity, such as in catalyzing a step in ethylene synthesis. Methods to assay for such activity are known in the art and are described more fully herein. Depending on context, “ACS3 activity” may refer to the activity of a native ACS3 polynucleotide or polypeptide. Such native activity may be modulated by expression of a heterologous ZmACS3 sequence as provided herein, for example when provided in a construct which downregulates the native ZmACS3.

The level of the ACS3 polypeptide may be measured directly, for example, by assaying for the level of the ACS3 polypeptide in the plant, or indirectly, for example, by measuring the ACS3 activity of the ACS3 polypeptide in the plant. Methods for determining the presence of ACS3 activity are described elsewhere herein or known in the art.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence, or its expression, in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984, all of which are herein incorporated by reference. See also, PCT Application Publication Numbers WO 98/49350, WO 99/07865, WO 99/25821 and Beetham, et al., (1999) Proc. Natl. Acad. Sci. USA 96:8774-8778, herein incorporated by reference.

The Zm-ACS3 nucleotide sequence set forth in SEQ ID NO: 10 can be used to generate variant nucleotide sequences having the nucleotide sequence of the open reading frame with about 70%, 75%, 80%, 85%, 90% or 95% nucleotide sequence identity when compared to the starting unaltered ORF nucleotide sequence of SEQ ID NO: 10. These functional variants are generated using a standard codon table. While the nucleotide sequence of the variant is altered, the amino acid sequence encoded by the open reading frame does not change.

Certain embodiments include plants having a transgene comprising a polynucleotide operably linked to a heterologous promoter that drives expression in the plant, wherein expression of the transgene results in modulation of expression of an ACS3 polynucleotide and/or polypeptide. Modulation of expression of other genes, including other ACS genes, may occur as a result of expression of the same transgene or a different transgene. Expression of the transgene may be constitutive or may be directed preferentially to a particular plant cell type or plant tissue type or may be inducible or otherwise controlled. Methods are provided to modulate plant growth and development, particularly plant response to stress, particularly abiotic stress, relative to a control plant, control plant cell or control plant part. The modulated growth or development may be reflected in, for example, higher growth rate, higher yield, altered morphology or appearance and/or an altered response to stress including an improved tolerance to stress. In certain embodiments, the stress is cold, salt or drought. In certain embodiments, yield is increased or maintained during periods of abiotic stress. Yield may be measured, for example, in terms of seed yield, plant biomass yield or recovery of other plant product or products. Seed set may be measured by, for example, seed number, total seed mass, average seed mass or some combination of these or other measures.

### Example 16

### Down-Regulation of Multiple ACS Genes

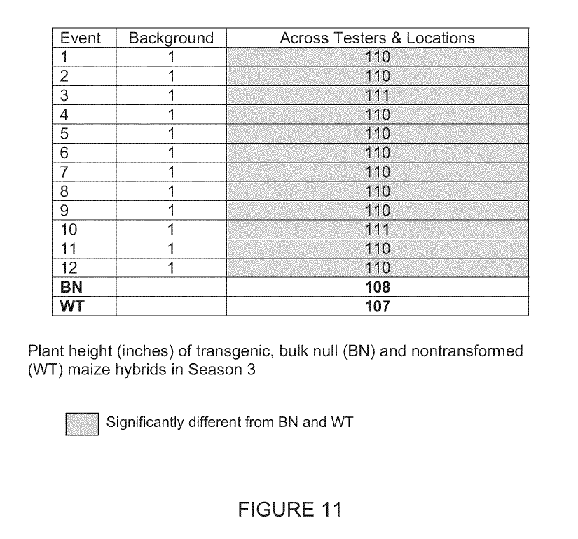

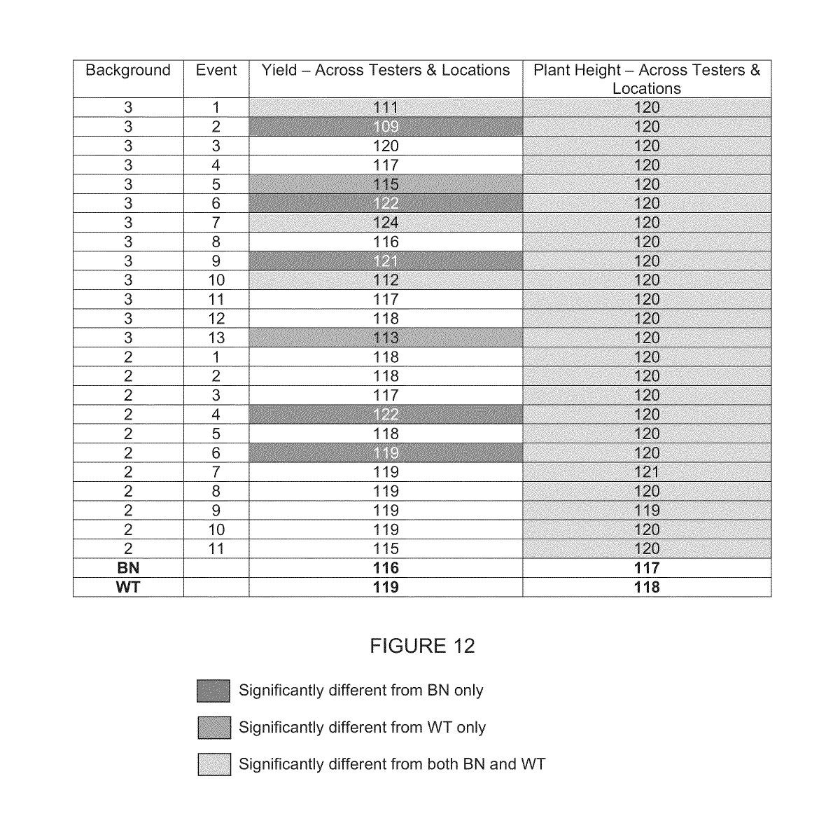

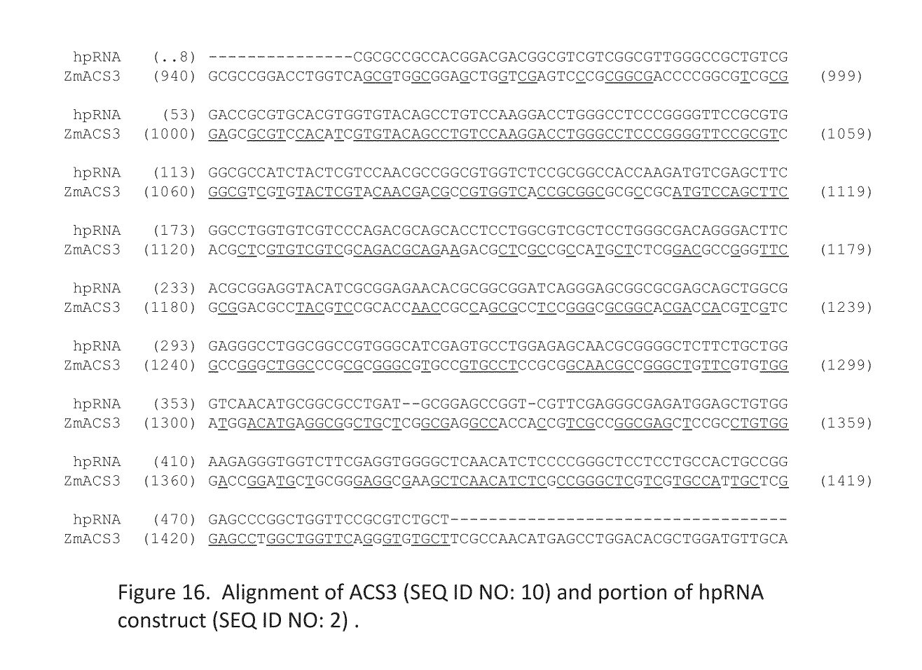

As shown in FIGS. 10-13, plants comprising a hpRNA comprising SEQ ID NO: 4 have shown consistent yield improvement under drought conditions across multiple environment and years, as well as consistent increase in height. This hpRNA comprises regions of identity to both ZmACS6 and the recently identified ZmACS3 gene. (See, FIG. 16; underlining indicates identical bases.) In one such region, 44 contiguous nucleotides of the ZmACS3 gene are identical to the hairpin sequence, indicating that the hpRNA of SEQ ID NO: 4 may down-regulate expression of ACS3 as well as ACS6. Further, ZmACS2 and ZmACS7 share contiguous 24- and 25-bp identity regions, respectively, with the hairpin sequence, which may result in downregulation of expression.

### Example 17

### Yield Evaluation of Transgenic Events in Two Backgrounds and Three Watering Regimes—Season 4

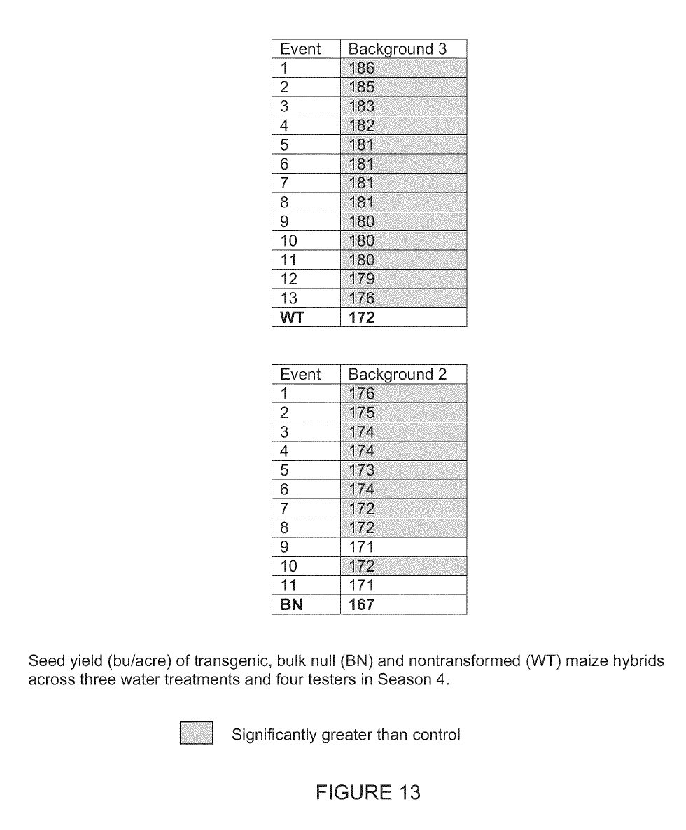

Hybrid maize plants, created by crossing four tester lines to plants of genetic background 2 or background 3 which comprised the recombinant polynucleotide of SEQ ID NO: 4 were evaluated for yield under three watering regimes in a randomized, nested experiment. Treatments were flowering stress (6 replications), grain-fill stress (4 replications) and well-watered conditions (4 replications). Flowering stress provided a 61% yield reduction, while grain-fill stress provided a 47% yield reduction, both relative to the well-watered yields. Controls were bulked null segregants as described above (BN), and nontransgenic hybrids of comparable base genetics (WT). Significance was determined at P<0.1 using BLUP (Best Linear Unbiased Predictor) analysis. Results are shown in FIG. 13. Events yielding significantly more than the control are shaded. Non-shaded data are not significantly different from the control values. For background #2, all thirteen events yielded significantly more than the control. For background #3, eleven of thirteen events yielded significantly more than the control; the other two had yield results not statistically different from the control.

### Example 18

### Reduction of ACC

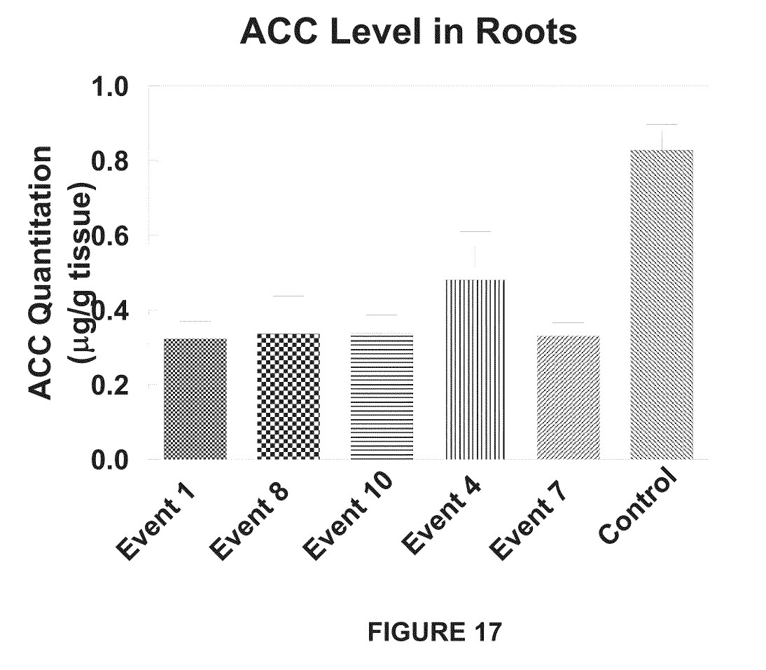

Downregulation of ACC synthase may be reflected in reduced levels of ACC. ACC may be assayed, for example, as described in Methods in Plant Biochemistry and Molecular Biology (1997) CRC Press, Ed. W. Dashek, at Chapter 12, pp. 158-159. Root tissue of maize plants at stage VT (Iowa State University Cooperative Extension Special Report No. 48 (1982, 1993)) were evaluated for ACC level. Plants of Background 1 comprising an ACS downregulation construct showed reduced ACC levels relative to the control; see FIG. 17.

### Example 19

### Reduced Expression of ACS6

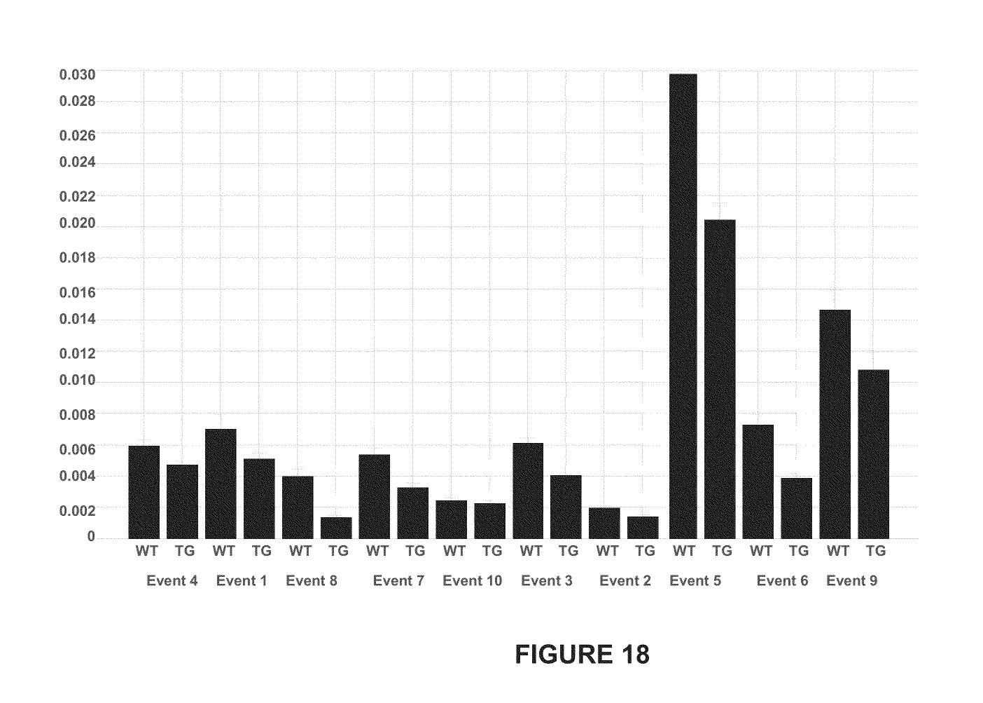

Using quantitative rtPCR methods generally as described elsewhere herein, maize plants transgenic for an ACS6 downregulation construct comprising SEQ ID NO: 4 were analyzed for ACS6 expression. Root tissue of flooded seedlings was sampled and results were as shown in FIG. 18. Each data point represents twelve plants in three replications at growth stage V3. Events are as indicated (TG) and data for wild type (WT) plants are provided as control.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications and/or other documents cited in this application are incorporated by reference in their entirety for all purposes to the same extent as if each individual publication, patent, patent application and/or other document were individually indicated to be incorporated by reference for all purposes.

What is claimed is:

1. An isolated nucleic acid comprising a promoter that functions in plants and further comprising a polynucleotide selected from the group consisting of SEQ ID NOS: 1, 2 and 4.

2. An isolated nucleic acid comprising a polynucleotide selected from the group consisting of SEQ ID NOS: 3, 5, 6 and 7.

3. The isolated nucleic acid of claim 1 comprising a promoter that functions in plants, wherein the polynucleotide comprises SEQ ID NO: 1 and SEQ ID NO: 2.

4. The isolated nucleic acid of claim 1 wherein said promoter is a constitutive promoter.

5. The isolated nucleic acid of claim 1, wherein expression of the nucleic acid results in the downregulation of the expression of one or more endogenous ACS genes in a plant cell.

6. A plant or plant cell comprising the isolated nucleic acid of claim 1.

7. A plant or plant cell comprising the isolated nucleic acid of claim 3.

8. A plant or plant cell comprising an expression cassette effective for reducing expression of at least one endogenous ACS gene, wherein said expression cassette comprises a promoter that functions in plants operably linked to a nucleic acid configured for RNA silencing or interference, wherein said nucleic acid comprises a polynucleotide of SEQ ID NO: 1 and/or SEQ ID NO: 2.

9. The plant cell of claim 8, wherein the plant cell is from a dicot or monocot.

10. The plant cell of claim 9, wherein the dicot or monocot is maize, wheat, rice, sorghum, barley, oat, lawn grass, rye, soybean, Brassica or sunflower.

11. A plant regenerated from the plant cell of claim 10.

12. The plant of claim 11, wherein the plant exhibits one or more of the following: increased drought tolerance, increased nitrogen utilization efficiency, increased seed yield, increased biomass yield, increased density tolerance and increased density tolerance, compared to a control plant.

13. A method of reducing ethylene production in a plant, the method comprising reducing the expression of one or more ACC synthase genes in the plant by expressing a transgenic nucleic acid comprising a nucleotide sequence selected from the group consisting of SEQ ID NO: 1, SEQ ID NO: 2, SEQ ID NO: 4, SEQ ID NO: 5, SEQ ID NO: 6 and SEQ ID NO: 7.

14. The method of claim 13, wherein the transformed plant exhibits one or more of the following: (a) a reduction in the production of at least one ACC synthase mRNA; (b) a reduction in the production of an ACC synthase; (c) a reduction in the production of ACC; (d) a reduction in the production of ethylene; (e) an increase in drought tolerance; (f) an increase in nitrogen utilization efficiency; (g) an increase in density tolerance; (h) an increase in plant height or (i) any combination of (a)-(h), compared to a control plant.

15. A method of increasing yield in a plant, the method comprising down regulating the expression of one or more ACC synthase genes in the plant by expressing a transgenic nucleic acid comprising a nucleotide sequence selected from the group consisting of SEQ ID NO: 1, SEQ ID NO: 2, SEQ ID NO: 4, SEQ ID NO: 5, SEQ ID NO: 6 and SEQ ID NO: 7.

16. A method of increasing drought tolerance in the absence of a yield penalty under non-drought conditions, the method comprising reducing endogenous ACS6 transcript levels or ACS6 activity.

17. An expression cassette consisting essentially of nucleotide sequences SEQ ID NO: 1 and SEQ ID NO: 2, wherein the nucleotide sequences are separated by an intervening polynucleotide.

18. The expression cassette of claim 17, wherein the intervening polynucleotide is a ZmAdh1 intron 1.

19. The expression cassette of claim 18, wherein the ZmAdh1 intron 1 sequence is bases 3791-4327 of SEQ ID NO: 3.

20. The plant of claim 8, wherein endogenous ACS transcript levels or ACS activity is reduced relative to a control plant.

21. The plant of claim 20, wherein the level or activity of ACC synthase is less than about 95% of that of the control plant.

22. The plant of claim 20, wherein the level or activity of ACC synthase is less than about 85% of that of the control plant.

23. The plant of claim 20, wherein the level or activity of ACC synthase is less than about 75% of that of the control plant.

24. The plant of claim 20, wherein the level or activity of ACC synthase is less than about 50% of that of the control plant.

25. The plant of claim 8, wherein the plant is maize, wheat, rice, sorghum, barley, oat, lawn grass, rye, soybean, sorghum, Brassica or sunflower

26. Seed of the plant of claim 8, wherein the seed comprises the expression cassette.
